(19) United States
(12) Patent Application Publication
(10) Pub. No.: US 2024/0398517 A1
(43) Pub. Date: Dec. 5, 2024

(54) IMAGE PROCESSING APPARATUS AND IMAGE PROCESSING METHOD USING SAME

(71) Applicant: MEDIT CORP., Seoul (KR)

(72) Inventor: Sung Bin IM, Seoul (KR)

(73) Assignee: MEDIT CORP, Seoul (KR)

(21) Appl. No.: 18/700,105

(22) PCT Filed: Oct. 12, 2022
(86) PCT No.: PCT/KR2022/015388

§ 371 Date: Apr. 10, 2024

(30) Foreign Application Priority Data
Oct. 12, 2021 (KR) 10-2021-0134839
Oct. 12, 2022 (KR) 10-2022-0130521

(51) Int. Cl. A61C 9/00; G16H 30/40

(52) U.S. Cl. CPC A61C 9/0053; G16H 30/40

(57) ABSTRACT

An image processing device according to the present disclosure comprises, a tool detachably coupled to one end of a scanner body, the scanner body having one end to which the tool is coupled, and a control unit configured to control an operation of the scanner body corresponding to the tool. An image processing method using an image processing apparatus, the method comprising, a tool attachment operation in which a user attaches a predetermined tool to a scanner body, a tool attachment/detachment identification operation in which a control unit determines attachment/detachment of the tool to/from the scanner body, a tool distinguishing operation in which the control unit distinguishes a type of the tool, and a scanner control operation in which the control unit controls an operation of the scanner body in response to the type of the tool.

### TECHNICAL FIELD

The present disclosure relates to an image processing apparatus and an image processing method using same. Specifically, the present disclosure relates to an image processing apparatus, in which an operation of a scanner body corresponding to a tool is performed by the tool attached to the scanner body, and an image processing method using same.

### BACKGROUND

Performance of optical apparatus may be sensitive to temperature and, specifically, an optical part of a three-dimensional scanner used for acquiring a three-dimensional model of a real object (in the present disclosure, referred to as “object”) may include a projector for emitting light toward the object and a camera for receiving light reflected from the object. The three-dimensional scanner (particularly, the optical part) needs to maintain high precision to acquire a three-dimensional model representing the object more accurately. Therefore, a user may need to perform error correction for the three-dimensional scanner, that is, calibration at predetermined intervals to keep the precision of the three-dimensional scanner high.

An example of a conventional process in which the user performs calibration of the three-dimensional scanner will be described. The user performs a scan program in a state in which a scanner body of the three-dimensional scanner is connected to a computing apparatus including a processor. When the scan program is performed, the user attaches a calibration tool to one end of the scanner body. Thereafter, the user selects a calibration button displayed on a user interface of the scan program to use a calibration function provided by the scan program.

When the user selects the calibration button, a calibration application is displayed in a pop-up form and the user performs calibration of the scanner body according to an indication of the calibration application.

In the process of performing the calibration as described above, the user may recognize that the calibration of the scanner body is required, through a predetermined notification (e.g., an overdue message displayed on the scan program) after executing the scan program and moving to a clinic chair to scan a patient's mouth. In this case, the user may need to move to the computing apparatus side from the chair again to perform the calibration and this process may cause inconvenience to the user. Further, after performing the calibration of the scanner body, in order to provide a hygienic scanning environment to the patient, the user had the inconvenience of having to change the gloves that had been worn or performing additional disinfection before scanning the patient's mouth.

### SUMMARY

To achieve the purpose of the present disclosure, an embodiment disclosed in the present disclosure provides an image processing apparatus in which a tool, which can be attached or detached to one end of a scanner body, is attached to the scanner body to perform an operation corresponding to a type of the attached tool, and an image processing method using same.

In addition, an embodiment disclosed in the present disclosure provides the image processing apparatus which may identify whether a tool is attached or detached through an attachment/detachment detection sensor provided to a scanner body, and the image processing method using same.

In addition, an embodiment disclosed in the present disclosure provides the image processing apparatus which acquires image data through an operation of an optical part built in a scanner body and distinguishes a type of tool based on the image data, and the image processing method using same.

In addition, an embodiment disclosed in the present disclosure provides the image processing apparatus which distinguishes a type of tool based on a distance between the tool and an attachment/detachment detection sensor which is provided to the scanner body and acquires the distance, and the image processing method using same.

In addition, an embodiment disclosed in the present disclosure provides the image processing apparatus which automatically performs a calibration application without a separate operation of a user when a calibration tool is determined to be attached, and the image processing method using same.

In addition, an embodiment disclosed in the present disclosure provides the image processing apparatus which performs a corresponding calibration process depending on a type of calibration tool attached to the scanner body, and the image processing method using same.

The technical problems solved by the present disclosure are not limited to the above technical problems and those skilled in the art will more clearly understand other technical problems not described above from the following description.

To achieve the objective as previously stated, the image processing device according to the disclosed embodiment of the present disclosure comprises, a tool detachably coupled to one end of a scanner body, the scanner body having one end to which the tool is coupled, and a control unit configured to control an operation of the scanner body in response to the tool.

In one embodiment, the tool may comprise a calibration tool formed into a shape with one side closed and configured to calibrate the scanner body, and a scanning tool formed into a shape with one side open.

In one embodiment, the scanner body may comprise, an attachment/detachment detection sensor formed on at least a portion of the scanner body and configured to identify whether the tool is attached to or detached from an attachment/detachment surface which couples the tool to the scanner body, a projector configured to emit a predetermined output light toward the tool, and at least one camera configured to receive reflection light generated by reflection of the output light and acquire at least one piece of image data from the reflection light.

In one embodiment, the projector may be configured to, when attachment or detachment of the tool is identified by the attachment/detachment detection sensor, emit the output light.

In one embodiment, the control unit may be configured to determine a type of the tool attached to the scanner body based on the image data acquired by the camera, and when the tool is determined as a calibration tool, perform a calibration operation.

In one embodiment, the control unit may be configured to, when a shape of the image data is determined to represent a pattern plate of the calibration tool, determine the type of the tool as the calibration tool and perform a calibration operation.

In one embodiment, the control unit may be configured to determine a type of the calibration tool as either a manual calibration tool or an auto-calibration tool based on the shape of the pattern plate, and perform a calibration operation corresponding to the type of the calibration tool.

In one embodiment, the control unit may be configured to, when the tool is attached to the scanner body, determine a type of the tool based on a distance between the attachment/detachment detection sensor and the tool, and when the tool is determined as a calibration tool, perform a calibration operation.

In one embodiment, the control unit may be configured to, when the tool is determined as a calibration tool, automatically execute a calibration application to be displayed on a display unit.

In one embodiment, the control unit may be configured to, when the tool is determined as a manual calibration tool among the calibration tool, control a guide message to be output on the display unit to guide a user to operate the manual calibration tool.

In one embodiment, the calibration tool may further comprise a pattern plate configured to be tiltable within a predetermined angle range based on a longitudinal direction of the scanner body, and wherein the pattern plate may comprise multiple targets.

In one embodiment, the multiple targets may comprise at least one calibration target for calibration of the scanner body, and multiple identification targets formed to be spaced apart from the calibration target, having a shape different from that of the calibration target, and used to identify a type of the calibration tool.

The image processing method using an image processing apparatus according to the embodiment disclosed in the present disclosure comprises a tool attachment operation in which a user attaches a predetermined tool to a scanner body, a tool attachment/detachment identification operation in which a control unit determines attachment/detachment of the tool to/from the scanner body, a tool distinguishing operation in which the control unit distinguishes a type of the tool, and a scanner control operation in which the control unit controls an operation of the scanner body in response to the type of the tool.

In one embodiment, in the tool attachment/detachment identification operation, the attachment/detachment of the tool may be identified by an attachment/detachment detection sensor formed on an attachment/detachment surface which is formed on at least a portion of the scanner body and configured to couple the tool to the scanner body.

In one embodiment, the tool distinguishing operation may comprise a light emission operation of emitting, when the attachment/detachment of the tool is identified in the tool attachment/detachment identification operation, predetermined output light from a projector built into the scanner body, an image data acquisition operation in which at least one camera built into the scanner body receives reflection light generated by reflection of the output light and acquires at least one piece of image data, and a tool determination operation of determining a type of the tool based on the image data.

In one embodiment, in the tool determination operation, the control unit may determine the type of the tool by determining a shape of a pattern plate built into a calibration tool through the image data.

In one embodiment, the control unit may be configured to, when the tool is attached to the scanner body, detect a type of the tool based on a distance between the attachment/detachment detection sensor and the tool.

In one embodiment, the method may further comprise a calibration application execution operation in which the control unit is configured to, when the control unit determines the tool as a calibration tool, automatically execute a calibration application to be displayed on a display unit.

In one embodiment, the control unit may be configured to, when the control unit distinguishes a type of the tool as a manual calibration tool among the calibration tool, control a guide message to be output on a display unit to guide the user to operate the manual calibration tool in the scanner control operation.

In one embodiment, the control unit may be configured to, when the control unit distinguishes a type of the tool as an auto-calibration tool among the calibration tool, control at least one selected from the group of the scanner body and the auto-calibration tool so that a calibration operation is automatically performed in the scanner control operation.

By using the image processing apparatus and the image processing method using the image processing apparatus according to an embodiment disclosed in the present disclosure, the user may have an advantage of minimizing inconvenience in calibrating the scanner body.

Further, since the attachment/detachment of the tool is identified by the attachment/detachment detection sensor of the scanner body and the type (e.g., a scanning tool and a calibration tool, or an auto-calibration tool and a manual calibration tool) of tool is determined based on the image data acquired by a camera, there is an advantage in that the type of tool may be quickly detected and an operation (e.g., calibration of the scanner body) of the scanner matching the type of tool may be performed without a separate operating process.

Further, since the type of tool is determined based on a distance between the tool and the attachment/detachment detection sensor, which is measured by the attachment/detachment detection sensor of the scanner body, there is an advantage in that the type of tool may be quickly determined and an operation of the scanner matching the type of tool may be performed without a separate operating process.

Further, when a control unit determines the type of tool to be a calibration tool, the control unit controls to automatically execute the calibration application to be displayed on a display unit and thus a process of requiring the user to execute a separate calibration application and select a calibration start button may be omitted, which provides an advantage of minimizing user inconvenience.

Further, the control unit distinguishes an auto-calibration tool and a manual calibration tool and performs control matching to a type of tool, thus providing an advantage of minimizing user inconvenience.

Further, when the auto-calibration tool is attached to the scanner body, the control unit applies a control signal to the auto-calibration tool so as to control a location of a pattern plate to be an initial location thereof, thus providing an advantage of performing accurate calibration of the scanner body through the auto-calibration tool.

### BRIEF DESCRIPTION OF DRAWINGS

The present disclosure may be easily understood through the following detailed description and the accompanying drawings, in which reference numerals refer to structural elements.

FIG. 1 is a view illustrating at least a portion of configurations of an image processing apparatus according to an embodiment of the present disclosure.

FIG. 2 is an example of a scanner body and a scanning tool detachably coupled to one end of the scanner body.

FIG. 3 is a cross-sectional view of a state in which a scanning tool is coupled to a scanner body.

FIG. 4 is a view illustrating an operation of photographing an object by an image processing apparatus according to the present disclosure.

FIG. 5 is an example of a scanner body and a calibration tool detachably coupled to one end of the scanner body.

FIGS. 6 and 7 are exploded views of a scanner body and a calibration tool detachably coupled to one end of the scanner body.

FIG. 8 is a cross-sectional view of a state in which a calibration tool is coupled to a scanner body.

FIG. 9 is a schematic view of a scanner body corresponding to a configuration of an image processing apparatus according to the present disclosure.

FIG. 10A is an example of image data acquired when a scanning tool is attached to an end of a scanner body and FIG. 10B is an example of image data acquired when a calibration tool is attached to an end of a scanner body.

FIG. 11 is an example of identification targets formed on a pattern plate of a calibration tool and a sign formed by the identification targets.

FIG. 12 is an example illustrating a process of performing calibration of a scanner body by using a calibration tool.

FIG. 13A is an example illustrating an auto-calibration tool, and FIG. 13B is an example illustrating three-dimensional information of identification targets which form a sign of the auto-calibration tool.

FIG. 14A is an example illustrating a manual calibration tool, and FIG. 14B is an example illustrating three-dimensional information of identification targets which form a sign of the manual calibration tool.

FIGS. 15A and 15B are examples illustrating a process of performing calibration of a scanner body by using a manual calibration tool.

FIG. 16 is an exemplary flowchart illustrating an image processing method using an image processing apparatus according to the present disclosure.

FIG. 17 is an example illustrating a tool distinguishing operation of an image processing method using an image processing apparatus according to the present disclosure.

FIG. 18 is another example illustrating a tool distinguishing operation of an image processing method using an image processing apparatus according to the present disclosure.

FIG. 19 is an example illustrating a scanner control operation of an image processing method using an image processing apparatus according to the present disclosure.

FIG. 20 is an example illustrating a calibration performance operation of an image processing method using an image processing apparatus according to the present disclosure.

BRIEF DESCRIPTION OF REFERENCE NUMERALS

| | |
|---|---|
| 1: Image processing apparatus | 100: Tool |
| 101: Scanning tool | 102: Calibration tool |
| 200: Scanner body | 500: External electronic apparatus |
| 510: Display unit | 610: Target |
| 611: Calibration target | 612: Identification target |
| 700: Sign | |
| S110: Tool attachment operation | S120: Tool attachment/detachment |
| | identification operation |
| S130: Tool distinguishing operation | S140: Scanner control operation |

### DETAILED DESCRIPTION

Hereinafter, some embodiments of the present disclosure will be described in detail with reference to exemplary drawings. In allocating reference numerals to components of individual drawings, the same component may have the same reference numeral even though the same component is displayed on different drawings. Furthermore, in describing some embodiments of the present disclosure, a detailed description of known elements or functions will be omitted if it is determined that the detailed description hinders understanding of the embodiments of the present disclosure.

In describing the components of an embodiment, terms such as first, second, A, B, (a), and (b) may be used. These are used solely for the purpose of differentiating one component from another and do not imply or suggest the essence, order or sequence of the components. In addition, unless defined otherwise, all technical and scientific terms used herein have the same meaning as commonly understood by a person of ordinary skill in the art to which the inventive concept belongs. It will be further understood that terms, such as those defined in commonly used dictionaries, should be interpreted as having a meaning that is consistent with their meaning in the context of the relevant art and should not be interpreted in an idealized or overly formal sense unless expressly defined herein.

The term “part (portion)” used in the specification may be implemented in software or hardware, and it is possible that multiple “parts” are implemented in one unit (element) or one “part” includes multiple components depending on the embodiments.

In describing the present disclosure, the expression “configured to” used in an embodiment of the disclosure may be used interchangeably with “suitable for,” “having the capacity to,” “designed to,” “adapted to,” “made to,” or “capable of” depending on a situation. The term “configured to” may not necessarily refer to “specifically designed to” in a hardware manner. Instead, in some situations, the expression “a system configured to” may refer to what the system is “capable of” operating in conjunction with other apparatuses or components. For example, the phrase “a processor configured to perform A, B, and C” may refer to a dedicated processor (e.g., an embedded processor) to perform the corresponding operation” or a generic-purpose processor (e.g., a CPU or application processor) capable of performing the corresponding operation by executing one or more software programs stored in a memory.

In an embodiment disclosed in the present disclosure, the three-dimensional scanner refers to an electronic apparatus configured to acquire an image related to an object. Specifically, the three-dimensional scanner referred to in the present disclosure may refer to a scanner configured to acquire an image related to an oral cavity, which is used for oral treatment. For example, the three-dimensional scanner in an embodiment of the present disclosure may include a hand-held intraoral scanner having a shape which may be inserted into an actual oral cavity of a patient.

Hereinafter, for convenience of explanation, the hand-held intraoral scanner having a shape which may be inserted into an actual oral cavity is referred to as the “three-dimensional scanner.”

In an embodiment disclosed in the present disclosure, an image may refer to an image (e.g., “an intraoral image”) representing an object included in an oral cavity. In this case, the object may include a tooth, a gingiva, and at least a partial portion of the oral cavity, and/or an artificial structure (e.g., an orthodontic apparatus including a bracket and a wire, an implant, a denture, a dental restoration including an inlay and an on-lay, and an orthodontic aid inserted into the oral cavity) insertable into the oral cavity. In addition, the object may also include an artifact related to the oral cavity, such as a plaster model, a crown, or the like. In addition, the orthodontic apparatus may include at least one of a bracket, an attachment, an orthodontic screw, a lingual orthodontic apparatus, and a removable orthodontic retainer.

In an embodiment disclosed in the present disclosure, the image may refer to an image showing the inside of the tool. That is, the image may refer to an image showing the inside of the calibration tool required for performing calibration of the three-dimensional scanner.

Furthermore, the image in an embodiment of the present disclosure may include a two-dimensional image with respect to the object or a three-dimensional model or three-dimensional image representing the object three-dimensionally.

Furthermore, the image in an embodiment of the present disclosure may refer to data required for presenting the object in two or three dimensions, for example, raw data or a raw image acquired from at least one camera. Specifically, the raw image may refer to data acquired to generate an intraoral image required for diagnoses and may include an image (e.g., a two-dimensional frame image) acquired by at least one camera included in the three-dimensional scanner when scanning the patient's oral cavity using the 3D scanner. In addition, the raw image may correspond to an unprocessed image and may refer to an original image acquired from the intraoral scanner.

Hereinafter, embodiments of the present disclosure will be described in detail with reference to the drawings.

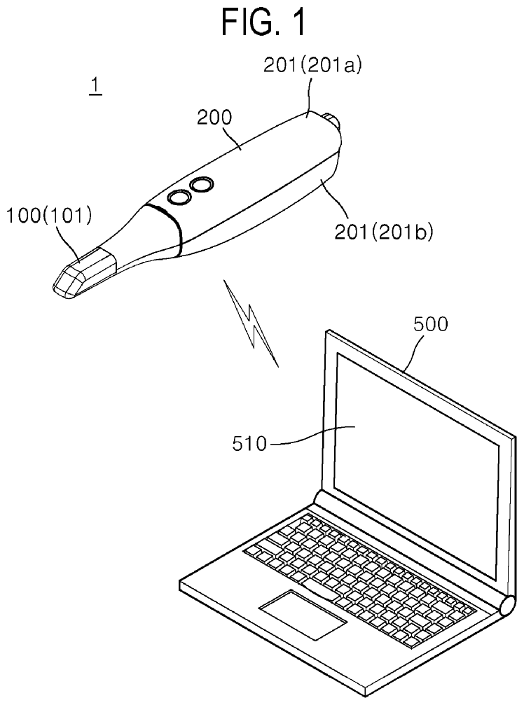

FIG. 1 is a view illustrating at least a portion of configurations of an image processing apparatus 1 according to an embodiment of the present disclosure. Referring to FIG. 1, the image processing apparatus according to the present disclosure includes a tool 100 and a scanner body 200. The tool 100 may be detachably coupled to one end of the scanner body 200. The tool 100 may be attached to the scanner body 200 or detached from the scanner body 200 to allow a predetermined operation of the scanner body 200 to be performed.

By coupling a specific type of tool 100 to one end of the scanner body 200, the scanner body 200 may acquire a three-dimensional model by scanning an object (not shown). According to an embodiment disclosed in the present disclosure, the tool 100 may correspond to a scanning tool 101, and when the scanning tool 101 is coupled to one end of the scanner body 200, a three-dimensional model may be acquired by the scanner body 200 scanning the object. Here, the object may refer to an object for which the user desires to acquire a three-dimensional model, and for example, the object may correspond to at least one of the inside of the patient's oral cavity, a negative model modeled after the inside of the patient's oral cavity, and a positive model acquired by pouring plaster on the negative model. The scanner body 200 may correspond to a component of the three-dimensional scanner, which is included in the three-dimensional scanner to scan the object and acquire the three-dimensional model.

The scanner body 200 may include a scanner body case 201. The scanner body case 201 may function to safely protect components built into the scanner body 200 from the external environment. By way of example, the body case 201 may be formed of a shape and material that is easy for the user to grasp by hand, but the shape and material of the body case 201 are not particularly limited.

For example, the body case 201 may include an upper case 201a and a lower case 201b, and a combination structure of the upper case 201a and the lower case 201b may allow the components built into the scanner body 200 to be protected from the external environment. However, the body case 201 may not be formed by the combination structure of the upper case 201a and the lower case 201b and may have a multi-stage combination structure or an integrated structure.

The scanner body 200 and an external electronic apparatus 500 may be connected to each other in communication. More specifically, the three-dimensional scanner included in the scanner body 200 may correspond to a medical apparatus for acquiring an image inside the oral cavity. The three-dimensional scanner insertable into an oral cavity, such as the intraoral scanner shown in FIG. 1, may be referred to as a hand-held intraoral scanner, a portable scanner, or the like.

More specifically, the intraoral scanner of the three-dimensional scanner may correspond to an apparatus which is inserted into the oral cavity and scans teeth in a non-contact manner to generate a three-dimensional model with respect to the oral cavity including at least one tooth. The intraoral scanner corresponding to one type of three-dimensional scanner may have a shape which may be inserted into or drawn out from the oral cavity and may scan the inside of the oral cavity of the patient by using at least one camera (e.g., an optical camera). In order to image a surface of at least one of objects, such as a tooth, a gingiva inside the oral cavity, an artificial structure (e.g., an orthodontic apparatus including a bracket, a wire, and the like, an implant, a denture, and an orthodontic aid inserted into the oral cavity) insertable into the oral cavity, and a plaster model, the three-dimensional scanner may acquire surface information with respect to the object as raw data.

Here, the raw data acquired by the three-dimensional scanner may include at least one image acquired by at least one camera included in the three-dimensional scanner. Specifically, the raw data may include at least one two-dimensional frame image acquired through intraoral scanning by the three-dimensional scanner. In this case, the “image frame” may be referred to as a “frame” or “frame data.”

The raw data acquired by the three-dimensional scanner may be transmitted to the external electronic apparatus 500 connected through a communication network.

The three-dimensional scanner may acquire a three-dimensional model or three-dimensional image generated based on the raw data acquired by the at least one camera. The acquired three-dimensional model or three-dimensional image may be transmitted to the external electronic apparatus 500.

The external electronic apparatus 500 may be connected to the three-dimensional scanner through a communication network and may receive data (e.g., image data) acquired by scanning the object from the three-dimensional scanner. The external electronic apparatus 500 may include any electronic apparatuses which may generate, process, display, and/or transmit an intraoral image based on the data transmitted from the three-dimensional scanner.

More specifically, the external electronic apparatus 500 may generate at least one of information required for oral diagnosis and an image representing the oral cavity based on the data received from the three-dimensional scanner, and display the generated information and image through a display unit 510. The display unit 510 may be a display apparatus visually displaying received data and/or data processed from the received data, and the display unit 510 may correspond to at least one of known display apparatuses (e.g., a monitor, a tablet, a screen, or the like).

For example, the external electronic apparatus 500 may include any electronic apparatuses which may generate, process, display, and/or transmit three-dimensional data or a three-dimensional image based on the image data received from the intraoral scanner.

For example, the external electronic apparatus 500 may include a computing apparatus, such as a smartphone, a laptop computer, a desktop computer, a PDA, a tablet PC, etc., but is not limited to the listed examples.

In addition, the external electronic apparatus 500 may exist in the form of a server (or server apparatus) for processing an image of an object (e.g., an intraoral image).

The external electronic apparatus 500 may store and execute dedicated software linked to the intraoral scanner. Here, the dedicated software may be referred to as a dedicated program or dedicated application. In case that the external electronic apparatus 500 operates in conjunction with the intraoral scanner, the dedicated software stored in the external electronic apparatus 500 may be connected to the intraoral scanner and receive data acquired through object scanning in real time. For example, dedicated software for processing data for each intraoral scanner product may exist. The dedicated software may perform at least one operation for acquiring, processing, storing, and/or transmitting a three-dimensional image of the object.

The three-dimensional scanner may transmit the raw data acquired by scanning the object to the external electronic apparatus 500 as it is. Thereafter, the external electronic apparatus 500 may generate a three-dimensional object image representing the object in three dimensions based on the received raw data. In addition, the external electronic apparatus 500 may generate a “three-dimensional intraoral image” by modeling an internal structure of the oral cavity in three-dimensions based on the received raw data, and therefore, data thus generated may be referred to as the “three-dimensional intraoral model.”

The external electronic apparatus 500 may receive a command through an input unit (not shown) electrically connected thereto. For example, the input unit may include at least one of known input apparatuses such as a keyboard, a mouse, and a scanner. By way of example, when the user inputs a calibration command through the input unit, a processor (not shown) built into the external electronic apparatus 500 may control an operation of the scanner body 200 so that the scanner body 200 performs the calibration process. A detailed description of the calibration process will be provided later.

Meanwhile, the aforementioned “three-dimensional scanner” or “intraoral scanner” includes the scanner body 200 and thus an operation of the “three-dimensional scanner” or “intraoral scanner” may correspond to an operation of the scanner body 200.

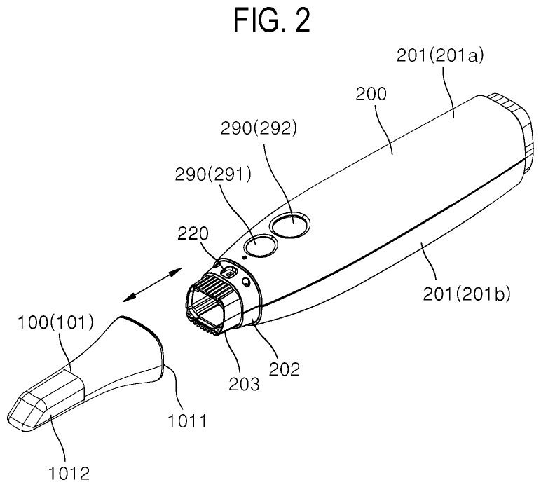

In addition, for example, the image processing apparatus according to an embodiment disclosed in the present disclosure may include a user interface, and the user interface may include an input apparatus including keys corresponding to a predetermined operation or command, or the like. For example, the input apparatus included in the user interface may include at least one button, a contact sensor, or the like. Alternatively, the user interface may include a voice recognition sensor and may receive a user's voice and recognize a user input corresponding to a predetermined operation or command based on the received user's voice. As shown in FIG. 2, the user interface included in scanner body 200 of the image processing apparatus may be formed with at least one button 290. In FIG. 2, it is exemplified that the user interface includes two buttons 291 and 292.

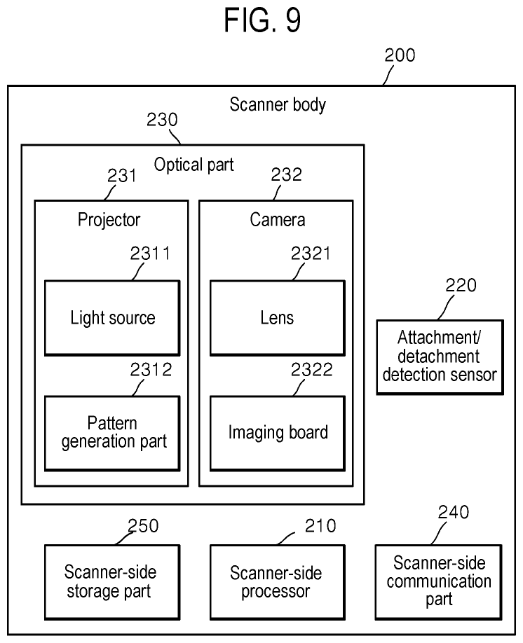

As another example, the user interface may be formed as a touch pad. In detail, the user interface may include a touch pad (not shown) to be coupled to a display panel (not shown). Here, a user interface screen may be output on the display panel. When a predetermined command is input through the user interface screen, the touch pad may detect information thereof and transmit the detected information to a scanner-side processor (210 in FIG. 9 described below). Thereafter, the scanner-side processor may interpret the detected information and recognize and execute the predetermined command input by the user.

More specifically, when the user interface includes the touch pad and the user touches a predetermined point of the user interface screen, the user interface may detect a location of the touched point. Thereafter, the detected location information may be transmitted to the scanner-side processor. The scanner-side processor may then recognize a user's request or command corresponding to a menu displayed on the detected location and execute the recognized request or command.

Hereinafter, a case in which the user interface includes multiple buttons 291 and 292 as shown in FIG. 2 to be described below will be provided as an example.

As shown in FIG. 2, the user interface may include a first button 291 among the buttons 290. When the user interface is formed as the first button 291, depending on a type in which the first button 291 is pressed, the user input corresponding to an operation of the first button 291 may correspond to at least one request. For example, a user operation with respect to the first button 291 may be distinguished as a double click, a long click, a single click, or a one-time click for a short period of time, and may be recognized as different requests depending on the distinguished user operation type.

By way of example, when the user briefly presses the first button 291 once, the scanner-side processor may recognize that a user input requesting to start scanning of the object is received. When the user presses the first button 291 once for a long time (or, for a configured time or longer), the scanner-side processor may recognize that a user input requesting termination of scanning the object is received. Alternatively, when the user double-clicks the first button 291, the scanner-side processor may recognize that a user input requesting transmission of image data corresponding to acquired images to the external electronic apparatus 500 is received.

As another example, different requests may be recognized in consideration of an operation state of the scanner body 200 when a user operation is performed on the first button 291. For example, when the first button 291 is briefly pressed once while the scanner body 200 performs scanning, the user input may be recognized as a request corresponding to the termination of the scanning. When the first button 291 is briefly pressed once in a state in which the scanner body 200 stops the scanning, the user input may be recognized as a request corresponding to a restart of the scanning.

Further, the user interface of the scanner body 200 of the image processing apparatus may include multiple buttons 291 and 292 corresponding to multiple requests, respectively. In this case, a corresponding request may be recognized depending on a selected button. For example, a second button 292 formed to be spaced a predetermined distance away from the first button 291 may serve as a cursor on the display unit 510. Accordingly, the user may operate an application (e.g., the calibration application) displayed on the display unit 510 by using the first button 291 and the second button 292 formed on the scanner body 200 without having a separate input apparatus.

Hereinafter, a tool 100 which is a component of the image processing apparatus according to an embodiment disclosed in the present disclosure will be described in detail.

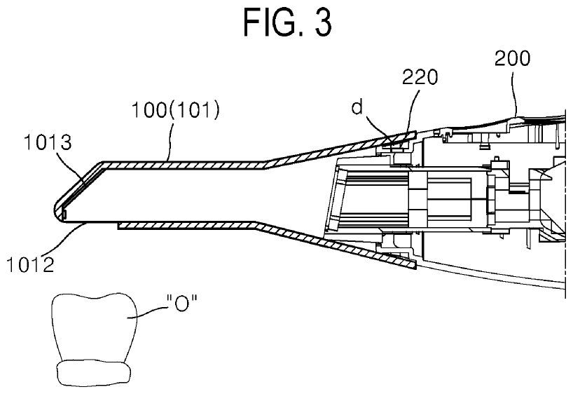

FIG. 2 is an example of a scanner body 200 and a scanning tool 101 detachably coupled to one end of the scanner body 200, and FIG. 3 is a cross-sectional view of a state in which the scanning tool 101 is coupled to the scanner body 200.

Referring to FIGS. 1 to 3, a type of the tool 100 which is a component of the image processing apparatus according to an embodiment disclosed in the present disclosure may vary depending on use. For example, in order to scan the object and acquire a three-dimensional model representing the object, the tool 100 coupled to an end of the scanner body 200 may correspond to the scanning tool 101. The scanning tool 101 may be formed to have a shape for guiding reflection light reflected from a surface and/or the inside of the object to the scanner body 200.

By way of example, the scanning tool 101 may be formed to have a shape including an opening on one side to guide light emitted toward the object from the scanner body 200 or guide reflection light reflected from the surface and/or the inside of the object to the scanner body 200. For example, the scanning tool 101 may include a scanning tool-side coupling part 1011 coupled to an end of the scanner body 200 and an opening part 1012 formed on one side of the other end opposite to the scanning tool-side coupling part 1011.

Meanwhile, the scanning tool 101 may include a light path change member 1013 to guide output light inside thereof toward the opening part 1012 or guide reflection light incident through the opening part 1012 toward the scanner body 200. The light path change member 1013 may reflect and/or refract light and the light path change member 1013 may correspond to an optical component, such as a mirror, a lens, or a prism.

Hereinafter, an operation of photographing the object in the scanner body 200 which corresponds to a component of the image processing apparatus 1 according to an embodiment disclosed in the present disclosure will be described in detail.

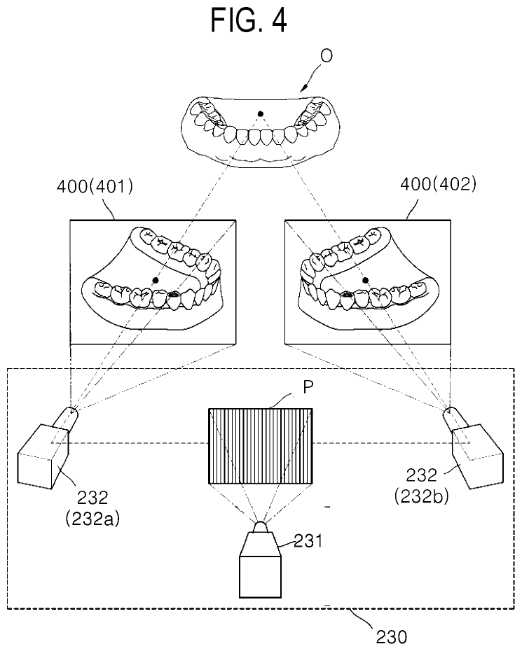

FIG. 4 is a view illustrating an operation of photographing an object by the image processing apparatus 1 according to the present disclosure.

Referring to FIG. 4, the scanner body 200 may include at least one camera 232 and the camera 232 may perform photographing with respect to the oral cavity and acquire at least one image. More specifically, the camera 232 may perform photographing with respect to the oval cavity to generate image data to be transmitted to an external electronic apparatus (not shown) (e.g., corresponding to 500 in FIG. 1).

Here, the image data generated in the camera 232 may correspond to at least one image itself, which is acquired by the at least one camera 232.

Alternatively, the camera 232 may generate image data corresponding to the acquired at least one image. Alternatively, the camera 232 may change a shape of the acquired at least one image to generate image data. Alternatively, the image data generated in the camera 232 may correspond to a three-dimensional image or a three-dimensional model representing the object in three dimensions based on multiple images acquired by the at least one camera 232. Hereinafter, for convenience of explanation, the “at least one camera 232” will be referred to as the “camera 232.” That is, the camera 232 may be referred to as one camera or multiple cameras.

The camera 232 may include at least one image sensor (not shown). Specifically, at least one camera included in the camera 232 each may include a lens 2321 and an image sensor (not shown). Here, the image sensor (not shown) may correspond to an apparatus which converts light incident through the lens 2321 into an electric signal to show an image for acquiring the image. For example, the image sensor may correspond to at least one of known image sensors such as a CCD sensor, CMOS sensor, or a color image sensor, but is not necessarily limited to the examples listed. The image sensor may be included in an imaging board 2322 disposed inside the camera, and the imaging board 2322 may be electrically connected to the lens 2321.

The camera 232 may acquire hundreds of images per second depending on a configured frames per second (FPS). Here, the image acquired by the camera 232 may correspond to a two-dimensional frame image. The FPS indicates the number of frame images acquired per second and may be referred to as a “frame rate.”

For example, when an operation frame per second (FPS) of the camera 232 is 100 FPS, the camera 232 may acquire 100 object images per second. For example, in case that the camera 232 of the scanner body 200 includes two cameras, an L camera (left camera) 232a and an R camera (right camera) 232b, 100 images may be acquired per second by the L camera 232a and 100 images may be acquired per second by the R camera 232b. Further, since the L camera 232a and the R camera 232b operate synchronized, the L camera 232a and the R camera 232b may concurrently acquire an L image and an R image, respectively.

As another example, in case that the camera 232 of the scanner body 200 includes one camera, 100 images may be acquired per second.

As yet another example, in case that the scanner body 200 performs image scanning in a confocal manner, each of the at least one camera included in the camera 232 may include a lens (not shown) configured to be movable to adjust a location of a focus and an image sensor (not shown) for acquiring an image based on light passing through the lens (not shown).

As still another example, in case that the scanner body 200 performs scanning in an optical triangle manner, each of the at least one camera included in the camera 232 may perform image scanning with respect to the object O to which the pattern is emitted.

Meanwhile, the camera 232 of the scanner body 200 may include, in addition to the at least one camera 232 for acquiring at least one image, an imaging board 2322 for acquiring image data corresponding to the at least one image.

Further, the imaging board 2322 may control the camera 232 for image scan. By way of example, the imaging board 2322 may configure a region of interest (ROI), an exposure time, a frame rate and/or the like of the camera 232.

Alternatively, the imaging board 2322 may generate image data corresponding to the at least one image acquired by the lens 2321. For example, the imaging board may convert a format of the at least one image acquired through the lens 2321 to generate image data corresponding to the at least one image.

Alternatively, the imaging board 2322 may generate image data corresponding to multiple images by encoding the at least one image acquired by the lens 2321.

In addition, in case that the camera 232 does not include the imaging board 2322, at least one of the aforementioned operations performed by the imaging board 2322 may be performed by a scanner-side processor 110 described below.

In an example shown in FIG. 4, a case in which the camera 232 included in the three-dimensional scanner 100 includes two cameras 232a and 232b and performs image scanning in an optical triangle manner is shown and described as an example.

Referring to FIG. 4, the three-dimensional scanner 100 may perform intraoral scanning using an optical triangle method. The optical triangle method may allow capturing an image by emitting light to the object, and acquiring three-dimensional data using the image of the object imaged with light. Specifically, by the optical triangle method, light is emitted to the object O and the light reflected by the object O is received so as to acquire an image with respect to the object O. Here, the three-dimensional data may include three-dimensional depth information with respect to the object O.

More specifically, in order to acquire the three-dimensional data with respect to a surface of the object O in an embodiment disclosed in the present disclosure, a structured light with stereo vision method using two cameras and a projector 231 outputting light may be used.

For example, in order to perform scanning by using the structured light with stereo vision method, the scanner body 200 may include an optical part 230, and the optical part 230 may include a projector 231 in addition to the camera 232 described above. Here, the projector 231 may emit, to the tool 100 side, output light having a pattern formed by at least one of a one-dimensional point and a two-dimensional line. Specifically, in order to scan an oral cavity which is one type of the object O, the projector 231 may emit output light into the oral cavity which is the object O by controlling of the scanner-side processor 210 described below. When the output light is emitted from the projector 231, two or more cameras 232a and 232b may receive reflection light reflected from the surface of the object O to which the output light has been emitted and acquire an image corresponding to the object O. Furthermore, a shape (or pattern) of the light output from the projector 231 may be changed and the light may have various shapes. In FIG. 4, a case in which the projector 231 outputs structured light p in a form of multiple lines is exemplified.

The scanner body 200 which is a component of the image processing apparatus 1 according to an embodiment disclosed in the present disclosure may emit the structured light p to the object O and the first camera 232a corresponding to a left field of view and the second camera 232b corresponding to a right field of view may acquire a first image 400 or 401 corresponding to the left field of view and a second image 400 or 402 corresponding to the right field of view, respectively. The scanner body 200 may continuously acquire two-dimensional frame images including the first image 400 or 401 and the second image 400 or 402 with respect to the object O. For example, in case that the camera 232 operates in 100 frames per second (FPS), the first camera 232a and the second camera 232b each may continuously capture 100 frame images per second. Here, the frame images acquired by the camera 232 may correspond to two-dimensional images corresponding to a resolution of the camera 232.

Further, the multiple frame images acquired by the two or more cameras 232a and 232b may be formatted in an imaging board (e.g., 2322 in FIG. 9 described below) into a different image format, for example, an HDMI format. Accordingly, an external electronic apparatus (e.g., 500 in FIG. 1) may receive HDMI data acquired by formatting the images acquired by the first camera 232a and the second camera 232b. In addition, the external electronic apparatus (e.g., 500 in FIG. 1) may reconstruct a three-dimensional frame representing a surface shape of the object O from the two-dimensional frame including the first image 400 or 401 and the second image 400 or 402. Specifically, depth information with respect to the object may be acquired based on the first image 400 or 401 and the second image 400 or 402 in which the structured light p is imaged together. Furthermore, a three-dimensional image or a three-dimensional model may be reconstructed based on the depth information.

The imaging board or the scanner-side processor may generate a three-dimensional image or a three-dimensional model with respect to the object based on the multiple frame images acquired by the two or more cameras 232a and 232b. Here, the scanner body 200 may transmit the generated three-dimensional image or three-dimensional model to the external electronic apparatus (e.g., 500 in FIG. 1) through a second communication network.

In FIG. 4, a case in which the scanner body 200 includes two cameras 232a and 232b and one projector 231 is exemplified. However, embodiments are not limited to the example shown in FIG. 4, and the scanner body 200 may include one camera and one projector. In case that the scanner body 200 includes one camera and one projector, the projector may concurrently function as a camera acquiring an image and a projector emitting structured light. Depending on various implementation methods of the present disclosure, the scanner body 200 may include multiple cameras and multiple projectors.

The scanner body 200 may move around the object O and scan the object O at predetermined time intervals (e.g., several ms to tens of ms) to acquire at least one image (e.g., multiple two-dimensional frames). The scanner body 200 or the external electronic apparatus (not shown) (e.g., 500 in FIG. 1) may acquire multiple three-dimensional frames from the multiple two-dimensional frames. For example, at least one image sensor which includes the camera 232 included in the scanner body 200 may each acquire tens to hundreds of two-dimensional frames per second. Specifically, the image sensor may acquire tens to hundreds of three-dimensional frames per second by using the multiple two-dimensional frames acquired from the scanner body 200. Here, each three-dimensional frame may be generated based on the multiple two-dimensional frames. For example, one three-dimensional frame may be generated based on dozens of two-dimensional frames.

In case that the external electronic apparatus (not shown) (e.g., 500 in FIG. 1) receives image data corresponding to the two-dimensional frames acquired from the scanner body 200, the external electronic apparatus may reconstruct a three-dimensional image or three-dimensional model with respect to the entirety of the object by combining or arranging the locations of multiple three-dimensional frames based on the received image data.

For example, each of the first camera 232a and the second camera 232b may acquire 100 or more two-dimensional frames per second. The first camera 232a and the second camera 232b may capture an image having a resolution of M*N. Here, M and N may have natural number values, M may represent the number of horizontal pixels of the acquired image, and N may represent the number of vertical pixels of the acquired image.

Hereinafter, for convenience of explanation, a case in which each of at least one image acquired by each of at least one image sensor (not shown) included in the camera 232 (e.g., the first camera 232a and the second camera 232b) corresponds to a two-dimensional frame formed by pixel values of 200 pixels in width and 200 pixels in height (i.e., M=200, and N=200) will be described and explained. In the example described above, although a case in which M and N have the same value has been exemplified, M and N may have different natural number values.

Further, one pixel value may be expressed as 8 bits. In this case, each of the frame images acquired by each of the first camera 232a and the second camera 232b may correspond to image data having a size or resolution of 200×200×8 bit=4,000 bytes.

A camera board may format a format of the multiple images acquired by the first camera 232a and the second camera 232b in a high-definition multimedia interface (HDMI) to generate image data (specifically, HDMI data). Here, the HDMI data may include 2K data, 4K data, or 8K data having the HDMI format. Here, the HDMI format may have an image frame form having a resolution defined in the HDMI standard and may have formats such as 1,920×1,080=2K resolution, 4,096×2,160=4K resolution, and 7,680×4,320=8K resolution.

Hereinafter, for convenience of explanation, a resolution of an image acquired from a camera (e.g., the first camera 232a and the second camera 232b) included in the scanner body 200 is referred to as a “first resolution” and a resolution of image data having the HDMI format is referred to as “second resolution.”

The first resolution may refer to a total resolution of at least one image acquired at the same time point by the camera 232 included in the scanner body 200. For example, if the intraoral scanner (e.g., 200 or 201) includes two cameras, the first camera 232a and the second camera 232b, the first image 400 or 401 and the second images 400 or 402 may be acquired at the same time point. In addition, in case that each of the two cameras 232a and 232b has a resolution of 200 pixels in width and 200 pixels in height, when two images acquired at once from the same time point by the two cameras 232a and 232b are combined in the horizontal direction, the image acquired by combining the two images may be expressed as having a resolution of 400 pixels in width and 200 pixels in height. That is, when two images acquired from the two cameras 232a and 232b included in the scanner body 200 are expressed as one image, the image having a resolution of 400 pixels in width and 200 pixels in height may be acquired. Hereinafter, for convenience of explanation, one image acquired by combining two images acquired from the two cameras 232a and 232b at the same time point will be referred to as a “raw image.”

In the example described above, the first resolution may correspond to a value acquired by multiplying 200 pixels in width by 200 pixels in height or a value acquired by multiplying 400 pixels in width by 200 pixels in height, and the second resolution may correspond to 2K, 4K, 8K, and the like.

As shown in the example described above, the scanner body 200, which is a component of the image processing apparatus 1 according to an embodiment disclosed in the present disclosure, may generate HDMI data including the pixel values of the frame images acquired from each of the first camera 232a and the second camera 232b as they are and transmit the HDMI data to an external electronic apparatus (not shown) (e.g., 500 in FIG. 1). Specifically, the scanner body 200 may format the pixel values of the frame images acquired from each of the first camera 232a and the second camera 232b in the HDMI format so that pixel values of frame images acquired from each of the first camera 232a and the second camera 232b are included as they are.

Hereinafter, a calibration tool 102, which is another type of the tool 100 of the image processing apparatus 1 according to an embodiment disclosed in the present disclosure will be described.

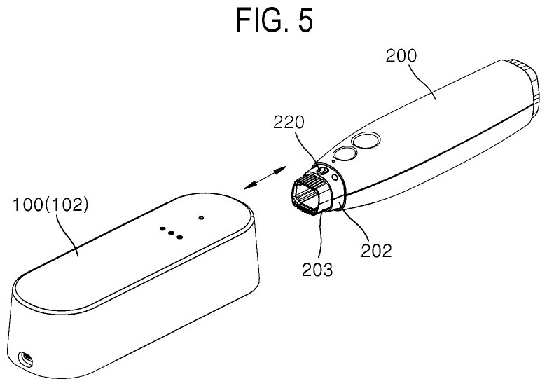

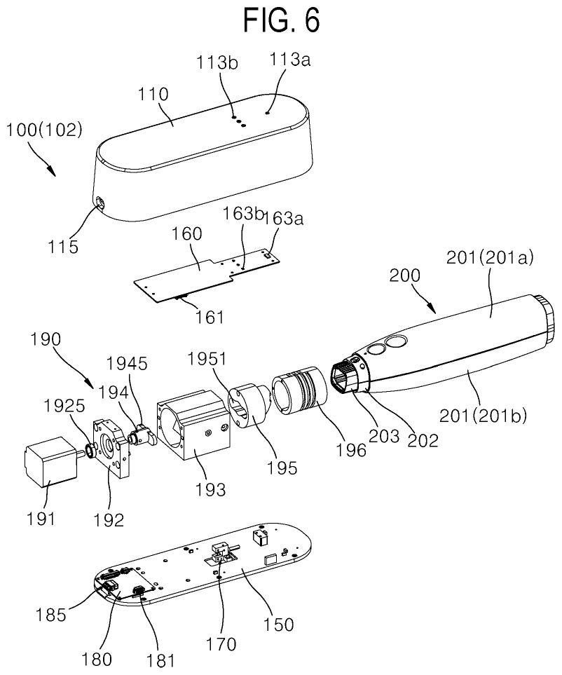

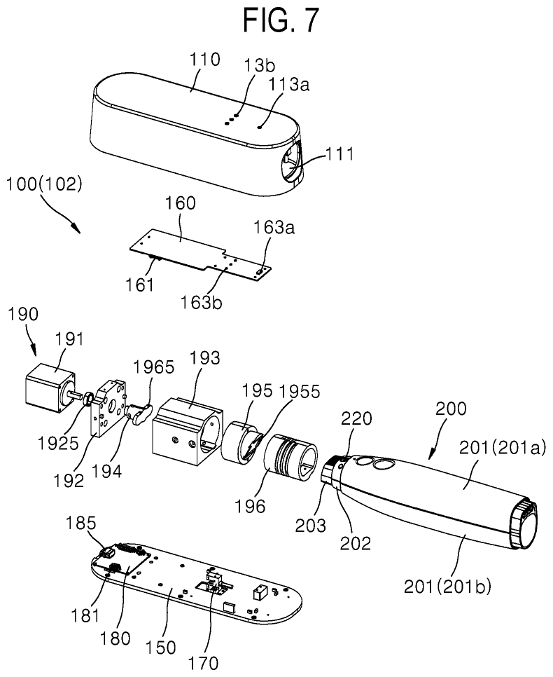

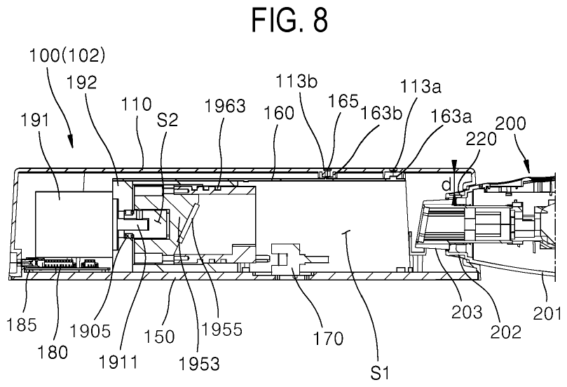

FIG. 5 is an example of a scanner body 200 and a calibration tool 102 detachably coupled to one end of the scanner body 200, FIGS. 6 and 7 are exploded views of the scanner body 200 and the calibration tool 102 detachably coupled to one end of the scanner body 200, and FIG. 8 is a cross-sectional view of a state in which the calibration tool 102 is coupled to the scanner body 200.

Referring to all of FIGS. 5 to 8, the tool 100, which is a component of the image processing apparatus 1 according to an embodiment disclosed in the present disclosure, may include the calibration tool 102. The calibration tool 102 may correspond to a component enabling calibration of at least some (particularly, the optical part) of components built in the scanner body 102, and may be detachably coupled to one end of the scanner body 200.

By way of example, the user may detach the scanning tool 101 which has been coupled to the scanner body 200 and attach the calibration tool 102, which is provided separately, thereto so as to perform calibration of the scanner body 200. When the user attaches the calibration tool 102 to the scanner body 200, an attachment/detachment surface 202, which is formed on at least a portion of the scanner body 200 and configured to couple the tool 100 to the scanner body 200, and a connection block 203 protruding from the attachment/detachment surface 202 by a predetermined length may be inserted into the calibration tool 102 and placed thereon. Since the attachment/detachment surface 202 and the connection block 203 are inserted into the calibration tool 102 and placed thereon, it is possible to stably calibrate the scanner body 200.

Hereinafter, a detailed configuration of the calibration tool 102 will be described more specifically. However, the detailed configuration of the calibration tool 102 may be changed, added, or removed as needed, and is not limited by the detailed description of the present disclosure described as an example.

In the image processing apparatus 1 according to an embodiment disclosed in the present disclosure, the calibration tool 102, which is one type of the tool 100, may include a calibration tool body 110 which is inserted into and seated on one end of the scanner body 200 in a state in which the scanning tool 101 is removed and includes a body insertion hole 111 formed thereon, a calibration pattern plate 1955 (hereinafter, referred to as a “pattern plate”) disposed inside the calibration body 110 to scan and correct the scanner body 200, and a pattern moving part 190 which automatically performs at least one of axis rotation movement and axis direction movement of the pattern plate 1955 inside the calibration tool body 110 when the scanner body 200 is coupled to the calibration tool body 110. Here, when one end of the scanner body 200 is inserted into the calibration tool body 110, the pattern plate 1955 may be disposed to face the camera provided inside the scanner body 200. In addition, the axis direction in which the pattern plate 1955 moves may correspond to a longitudinal direction of the scanner body 200. The calibration tool 102 may include a tool which may automatically perform the axis rotation movement and the axis direction movement of the pattern plate 1955.

For example, the calibration tool body 110 may have a flat lower surface so as to be more stably supported and seated on a desk or table where calibration is performed. In addition, the calibration tool body 110 may have a lower surface having an area relatively smaller than that of an upper surface, but is not limited thereto. The upper and lower surfaces of the calibration tool body 110 may be formed to have rounded edges as a whole. As another example, the calibration tool body 110 may have a cylindrical shape as a whole.

An emission path of the output light emitted from the projector (e.g., 231 in FIG. 4 described above and FIG. 9 described below) of the scanner body 200 may be provided inside the calibration tool body 110 and in particular, the emission path of the output light and the reflection light may be provided in a dark room form so as not to be affected by external light. That is, the calibration tool 102 is formed into a shape having one side closed, and when one end of the scanner body 200 is inserted into and seated on the scanner body insertion hole 111, the inside of the calibration tool 102 forms a dark room, and the output light emitted from the projector 231 of the scanner body 200 and the reflection light reflected from the pattern plate 1955 exist inside the calibration tool 102.

The calibration tool body 110 may include a main printed circuit board 150 which configures the lower surface of the calibration tool body 110 and shields the inside thereof in a dark room form.

On the main printed circuit board 150, a scanning location detection part to be described below may be mounted and disposed and a power supply line (not shown) for supplying external power may be printed. However, the lower surface of the calibration tool body 110 does not necessarily need to be provided in the form of a PCB, such as the above-described main printed circuit board 150, and may be provided in various other forms as long as long as it is possible to supply power to a specific component (e.g., scanning location detection part).

Further, the main printed circuit board 150 may include a motor control part (not shown) mounted and disposed in a MICOM form for controlling an operation of a driving motor 191 among configurations of the pattern moving part 190 to be described below.

A motor PCB 180 for controlling operation and supplying power of the driving motor 191 among configurations of the pattern moving part 190 may be provided in a sub-PCB form inside the calibration tool body 110. The motor PCB 180 may include an external power connector 185 capable of supplying external power through a wired connection and an internal power connector 181 mounted and disposed thereon for power connection to a display PCB 160 described below.

In addition, the display PCB 160 for displaying an operating state of the calibration tool 102 may be further provided inside the calibration tool body 110.

The display PCB 160 may be disposed to be in close contact with an inner upper surface of the calibration tool body 110, and a power on/off indicator lighting 163a and an operating state indicator lighting 163b may be provided in an LED form on an upper surface of the display PCB 160. In addition, a power supply connector 161 for wired connection with the internal power connector 181 provided on one side of the motor PCB 180 may be provided on one side of an edge of the display PCB 160 as described above.

A power on/off indicator hole 113a and an operation status indicator hole 113b, which allow the light emitted from the power on/off indicator lighting 163a and the operation status indicator lighting 163b to be transmitted to the outside, may be formed in the upper surface of the calibration tool body 110.

Here, light of the operating state indicator lighting 163b mounted and disposed on the display PCB 160 may be emitted to the outside through the operating state indicator hole 113b via a light guide 165 made of a transparent material.

The scanner body insertion hole 111 formed on one side of the calibration tool body 110 in the longitudinal direction may have a form to which the connection block 203 provided at one end (e.g., a front end) of the scanner body 200 in a state in which the scanning tool 101 is removed may be inserted and seated. When the scanning tool 101 is detached from the scanner body 200, the connection block 203 may be exposed to protrude from one end of the scanner body case 201 by a predetermined length.

The calibration tool body 110 may further include an illuminance sensor 169 inside thereof to detect predetermined light. The illuminance sensor 169 may detect that predetermined output light is emitted from the projector 231 into an inner space of the calibration tool body 110 when the scanner body 200 operates. The illuminance sensor 169 may inform the control unit of a time point when the driving motor 191 among configurations of the pattern moving part 190 to be described below may be operated by sensing the output light.

In this case, the illuminance sensor 169 may be mounted and disposed on a lower surface of the display PCB 160 to more accurately measure the output light of the inner space of the calibration tool body 110, but is not limited thereto.

The calibration tool body 110 may further include the scanning location detection part for detecting a location of the pattern plate 1955 inside thereof. The scanning location detection part may detect a location of a mounting block 195 to which the pattern plate 1955 is coupled, thereby providing information so that the control unit may calculate a required distance value and rotation angle value when performing calibration. The scanning location detection part may identify whether the pattern moving part 190 is restored to an initial location at which calibration is performed.

The scanning location detection part performing the function may include, for example, a photo sensor part and a Hall sensor part, but is not limited thereto.

For example, when the scanning location detection part corresponds to a photo sensor part, the photo sensor part may include a photo sensor 170 fixed to the bottom surface of the calibration tool body 110 and a detection lead 175 which is coupled to a moving block 196 or the mounting block 195 to which the pattern plate 1955 is coupled, and rotated and rectilinearly moved.

The detection lead 175 may be coupled to a front edge portion of the moving block 196 or the mounting block 195 among configurations of the pattern moving part 190 and linked and moved together when the moving block 196 or the mounting block 195 performs an axis rotation and/or an axis direction rectilinear movement. In case that the detection lead 175 moves and is inserted into the photo sensor 170, the photo sensor 170 may detect the detection lead 175, detect locations of the moving block 196 and the mounting block 195 to which the detection lead 175 is coupled, and detect a location of the pattern plate 1955 coupled to the mounting block 195. The control unit may calculate a separation distance between the pattern moving part 190 or the pattern plate 1955 and the photo sensor 170 and a rotation angle value through information acquired from the photo sensor part.

As another example, when the scanning location detection part corresponds to a Hall sensor part, the Hall sensor part may include a Hall sensor (not shown) fixed to the calibration tool body 110 and a detection magnet (not shown) which rotates and moves rectilinearly linked to the pattern plate 1955.

The detection magnet may be configured to interact with the Hall sensor through magnetism. The detection magnet may be provided at a front edge portion of the moving block 196 or the mounting block 195 among configurations of the pattern moving part 190 and interlocked and moved together when the moving block 196 or the mounting block 195 performs an axis rotation and/or an axis direction rectilinear movement. In case that the detection magnet moves and is detected by the Hall sensor, the Hall sensor may detect a location of the detection magnet and detect a location of the pattern plate 1955 coupled to the mounting block 195. The control unit may be configured to relatively measure a separation distance between the pattern moving part 190 or the pattern plate 1955 and the Hall sensor and a rotation angle value through information acquired from the Hall sensor part.

When an operation of the scanner body 200 is detected after the scanner body 200 is inserted and seated on the calibration tool body 110 by the illuminance sensor 169, the scanning location detection part may identify the detected location (more specifically, a rotation angle state and a current location of the pattern plate 1955) of the pattern moving part 190 and suggest a reference value for restoring to the initial location if not in the initial calibration location.

The calibration tool main body 110 configured as described above may include a pattern moving part 190 provided inside thereof to move in a horizontal direction. Hereinafter, the “horizontal direction” is defined as indicating a direction parallel to an upper surface of a table on which the calibration tool body 110 is mounted and may be understood as including the longitudinal direction of the calibration tool body 110 and the longitudinal direction of the scanner body 200.

The pattern moving part 190 may be provided to allow the pattern plate 1955 to rotate in the horizontal direction (i.e., the longitudinal direction of the calibration tool body 110) in which one end of the scanner body 200 is inserted and seated as an axis and to reciprocate in the axis direction and may function to automatically move the pattern plate 1955 to enable calibration. Here, the pattern moving part 190 may cause the pattern plate 1955 to simultaneously perform an axis rotation and movement in the axis direction, and while the pattern plate 1955 moves, an angle between optical axes of the output light emitted from the pattern plate 1955 and the scanner body 200 to the pattern plate 1955 may be maintained.

The pattern moving part 190 may be electrically operated by external power supplied through the above-described main printed circuit board 150 or internal power provided in a form like a rechargeable battery inside the calibration tool body 110. In case that the internal power is provided as a rechargeable battery inside the calibration tool body 110, the rechargeable battery may be charged with power in a wired or wireless manner.

The pattern plate 1955 has a predetermined pattern printed or provided for calibration and is provided to perform calibration while being moved linked with the rotation or rectilinear movement of the pattern moving part 190. The pattern plate 1955 may be formed to be tiltable within a predetermined angle range based on the longitudinal direction of the scanner body 200.

The pattern plate 1955 may be disposed to be inclined on a front surface of the mounting block 195 to be described below. To this end, the front surface of the mounting block 195 may be inclined to have a predetermined inclination angle.

Here, the inclination angle of the pattern plate 1955 may be configured to be 40 degrees or more and less than 50 degrees based on the horizontal direction (the longitudinal direction of the scanner body 200). In this case, there is a disadvantage in that, when the pattern plate 1955 is provided to be orthogonal (i.e., 90 degrees) with respect to the horizontal direction, each pattern 255 formed on the pattern plate 1955 has the same depth information (or height information) on the same surface, and thus, in an embodiment of the present disclosure, the pattern plate 1955 is designed to increase the calibration effect by inclining the pattern plate 1955 at a predetermined angle with respect to the horizontal direction.

The pattern moving part 190 may include the driving motor 191 that may be electrically operated and has a rotation shaft, a fixed block 193 fixed to the inside of the calibration tool body 110 and having moving guide holes opened to one side and the other side thereof, and the mounting block 195 to which the pattern plate 1955 is coupled and which is disposed in a moving guide hole in the fixed block 193.

The driving motor 191 may be provided to be fixed adjacent to one end side of the inner space of the calibration tool body 110 where the scanner insertion hole 111 is formed and the other end side opposite to the one end, and to be parallel or coincident with the optical axis. Therefore, it is natural that the axis direction of the pattern plate 1955 moved by the driving of the driving motor 191 may be interpreted as coincident with or parallel to the optical axis of light emitted from the scanner body 200 to the pattern plate 1955. The driving motor 191 may be electrically driven using external power or internal power.

In addition, the pattern moving part 190 may include a transfer block 194 disposed between the driving motor 191 and the mounting block 195 to transfer a rotational force of the driving motor 191 to the mounting block 195.

Here, the transfer block 194 may transfer the rotational force transferred from a rotation shaft 1911 of the driving motor 191 to the mounting block 195, so that the mounting block 195 is configured to rotate within a moving guide hole of the fixed block 193 so as to rotate the pattern plate 1955 disposed to be inclined on an inclined surface of a reception terminal of the mounting block 195 formed in an inclined manner.

The transfer block 194 may include a coupling terminal axially coupled to the rotating shaft of the driving motor 191 and a rotation wing terminal 1945 which is provided at a front end of the coupling terminal and is inserted into a rotation interference hole 1951 formed in the mounting block 195 to cause rotation interference.

Here, when it is assumed that the coupling terminal is formed to have a circular vertical cross section, the rotation wing terminal 1945 may be formed in a wing shape extending to one side and/or the other side farther than an outer circumferential surface of the coupling terminal. When the transfer block 194 is rotated by the rotation wing terminal 1945, it interferes with the mounting block 195, so that the mounting block 195 may be rotated in linkage with the rotation shaft of the driving motor 191.

The rotation interference hole 1951 formed in the mounting block 195 and the rotation wing terminal 1945 of the transfer block 194 may be configured to have vertical cross sections corresponding to each other. In addition, the rotation interference hole 1951 formed in the mounting block 195 may be formed in a shape that interferes with the rotation direction of the rotation wing terminal 1945 and does not interfere with the horizontal direction of the rotation wing terminal 1945.

As such, the rotation interference hole 1951 and the rotation wing terminal 1945 are formed to have vertical cross sections corresponding to each other and have structures that do not interfere with each other in the horizontal direction (i.e., the axis direction), so that the mounting block 195 may be reciprocally moved in the axis direction by the moving block 196 to be described below.

In addition, it is preferable that the rotation interference hole 1951 is formed so that the depth thereof formed in the horizontal direction from the front end of the rotation wing terminal 1945 is greater than or equal to at least a horizontally movable distance of the mounting block 195. This is because the depth of the rotation interference hole 1951 in the horizontal direction is larger than at least the movable distance of the pattern plate 1955, so as to prevent the movement distance from being limited by interference between the mounting block 195 and the transfer block 194.

Here, the rotation shaft 1911 of the driving motor 191 may be stably supported via a center block 192 provided to be supported on the inner surface of the calibration tool body 110, and the transfer block 194 may be axially fixed to the front end of the rotation shaft 1911 of the driving motor 191. The rotation shaft 1911 of the driving motor 191 may be supported for axis rotation by a rotation bearing 1925 interposed in a through-hole 1921 of the center block 192.

Meanwhile, the pattern moving part 190 may further include the moving block 196 which is provided to be linked with the mounting block 195, and rectilinearly moves the mounting block 195 in the horizontal direction by interference with the fixed block 193.

The moving block 196 serves to reciprocate the pattern plate 1955 in the horizontal direction (axis direction) while being rotated in linkage with the mounting block 1950 to which the pattern plate 1955 is coupled.

To this end, the moving block 196 may rotate and rectilinearly move inside the moving guide hole, and the outer circumferential surface of the moving block 196 may include a rotation guide groove formed thereon, in which a front end of a guide member protruding into the moving guide hole of the fixed block 193 is engaged.

The rotation guide groove may be formed on the outer circumferential surface of the moving block 196 and may be machined to be grooved in a spiral shape with a predetermined pitch interval so that the moving block 196 makes at least three rotations.

A pair of guide members may be provided to be spaced apart at intervals of 180 degrees with respect to the center of the moving guide hole of the fixed block 193, but the number and spacing are not limited thereto. One end of the guide member may be inserted into the rotation guide groove provided in a spiral shape.

The mounting block 195 is provided to perform linked rotation with the moving block 196 and reciprocating rectilinear movement in the horizontal direction, and the guide member fixed to the fixed block 193 is provided to be engaged into the rotation guide groove formed on the outer circumferential surface of the moving block 196 which is rotating, so that the rectilinear movement distance of the pattern plate 1955 may be determined according to an amount of rotation of the moving block 196.

However, it is not necessary that the guide member protrudes into the fixed block 193 and the rotation guide groove is provided on the outer circumferential surface of the moving block 196. That is, as described above, if the pattern plate 1955 has a structure capable of rectilinearly moving in the horizontal direction according to the amount of rotation of the moving block 196, the opposite structure may be adopted as well.

More specifically, although not shown in the drawings, the rotation guide groove which allows the guide member to be engaged on an inner circumferential surface of the moving guide hole may be formed, and the guide member may be provided on the outer circumferential surface of the moving block 196.

Meanwhile, the guide member may include a guide bolt, a portion of which is inserted into the rotation guide groove. However, the guide member does not necessarily have to be provided as a guide bolt, and the guide member may correspond to a ball plunger in which a bearing ball is installed. The case in which the guide member is provided as a ball plunger has an advantage of minimizing frictional force due to the rotation of the moving block 196.

On the other hand, contrary to the above description, the calibration tool 102 according to another embodiment of the present disclosure may not include the driving motor, and may include a fixed case for connecting and fixing the scanner body 200, a rotation case rotatably provided with respect to the fixed case, and a moving means provided inside the rotation case and transferring rotation force of the rotation case to rotate and/or rectilinearly reciprocate the pattern plate 1955. That is, the calibration tool 102 may include a manual calibration tool. By way of example, the user may rotate and/or rectilinearly reciprocate the pattern plate 1955 by manipulating the rotation case of the manual calibration tool. As another example, the moving means for rotating and/or rectilinearly reciprocating the pattern plate 1955 may be provided in the form of a dial lever, and the user rotates the dial lever in one direction to rotate and/or rectilinearly reciprocate the pattern plate 1955.

Hereinafter, a detailed configuration of the scanner body 200 which corresponds to a component of the image processing apparatus 1 according to an embodiment disclosed in the present disclosure will be described in detail.

FIG. 9 is a schematic view of a scanner body 200 corresponding to a configuration of the image processing apparatus 1 according to the present disclosure.

Referring to FIGS. 2, 3, 8, and 9, the scanner body 200, which is one component of the image processing apparatus 1 according to an embodiment disclosed in the present disclosure, may include a scanner-side processor 210.

The scanner-side processor 210 may perform at least one control so that the scanner-side processor 210 may perform a desired operation. Specifically, the scanner-side processor 210 may control an operation of photographing (or scanning) with respect to the object, an operation of acquiring an image with respect to the object, and/or an operation of transmitting data (e.g., image data) corresponding to the acquired image. When it is described that the scanner-side processor 210 performs a predetermined operation, the description may indicate not only the case of directly performing the above-described operation by executing at least one control in the scanner-side processor 210, but also the case of controlling other components to perform the above-described operation.

More specifically, the scanner-side processor 210 may include a RAM (not shown) for storing a control signal or data input from the outside of the scanner body 200 or used as a storage area corresponding to various operations performed in the scanner body 200, a ROM (not shown) in which a control program for controlling the scanner body 200 and/or multiple pieces of control-related information are stored, and at least one processor (not shown) (hereinafter, referred to as an “internal processor”) for executing at least one control. The scanner-side processor 210 may be implemented to have a form including at least one internal processor internally and a memory element (e.g., a RAM, a ROM, and the like) for storing at least one of a program, an instruction, a signal, and data to be processed or used by the internal processor.

Further, the scanner-side processor 210 may include a graphic processing unit (not shown) for graphic processing corresponding to a video. The processor 210 may be implemented as a system on chip (SoC) including a core (not shown) and a GPU (not shown) integrated therein. The scanner-side processor 210 may include multiple cores of more than a single core. For example, the scanner-side processor 210 may include a dual-core, a triple-core, a quad-core, a hexa-core, an octa-core, a deca-core, a dodeca-core, a hexadecimal-core, and the like.

In addition, the scanner-side processor 210 may include a designable logic device and a field-programmable gate array (FPGA) which is a semiconductor device including a programmable internal circuit and may perform high-speed image processing using the FPGA.

In detail, the scanner-side processor 210 may acquire image data corresponding to at least one image acquired by the at least one camera 232, control a control signal, which is related to at least one of the operation of acquiring the at least one image and a transmission operation, to be transmitted or received to or from an external electronic apparatus through a scanner-side communication part 240 performing wired or wireless communication, and control the image data to be transmitted to an external electronic apparatus.

The image processing device 1 according to an embodiment disclosed in the present disclosure may include a control unit. The control unit may control an operation of the scanner body 200 in response to the tool 100 to be coupled to the scanner body 200. The control unit may correspond to the scanner-side processor 210 built into the scanner body 200 or a processor of the aforementioned external electronic apparatus (e.g., 500 in FIG. 1).

The scanner body 200 of the image processing apparatus 1 according to an embodiment disclosed in the present disclosure may include an attachment/detachment detection sensor 220. The attachment/detachment detection sensor 220 may be formed on at least a portion of the scanner body 200 to identify attachment/detachment of the tool 100. For example, the attachment/detachment detection sensor 220 may be formed on at least a portion of an attachment/detachment surface 202 formed at one end of the scanner body 200 in order to identify whether the tool is attached or detached. The attachment/detachment detection sensor 220 may correspond to a proximity sensor for identifying that the tool 100 is attached or detached (mounted or removed) to or from the scanner body 200, but is not necessarily limited to the specified example. A process of identifying the type of the tool 100 by operating the optical unit 230 to be described below by the attachment/detachment detection sensor 220, and a process of identifying the type of the tool 100 based on information acquired by the attachment/detachment detection sensor 220 will be described below.

The scanner body 200 of the image processing apparatus 1 according to an embodiment disclosed in the present disclosure may include an optical part 230, and the optical part 230 may include a projector 231 for emitting predetermined light toward an object and a camera 232 for acquiring image data.

The projector 231 may emit predetermined output light and/or a pattern to the object so that the camera 232 may acquire a color and a shape of image data representing the object. The projector 231 may include a light source 2311, and the light source 2311 may operate to acquire a color and a shape of image data. For example, the light source 2311 may include a red light source, a green light source, and a blue light source, and a first color image, a second color image, and a third color image may be acquired by simultaneously or sequentially operating the light sources. The color of the image data may be acquired by combining the color images. However, the light source 2311 is not limited to the listed examples, and may include a light source conforming to various known color acquisition methods.

The light may be emitted to the object with a specific pattern, and the light emitted to the object with the specific pattern may correspond to structured light. A camera module 132 may acquire the shape (i.e., a three-dimensional shape of the object) of image data by acquiring an image of the object to which the structured light is emitted.

The pattern may be generated by a pattern generation part 2312 of the projector 231. Light generated by the light source 2311 may pass through the pattern generation part 2312, and the light passing through the pattern generation part 2312 may be converted into output light corresponding to a shape of the pattern generated by the pattern generation part 2312. For example, the pattern generation part 2312 may correspond to at least one of a pattern mask and a digital micromirror device (DMD), but is not necessarily limited to the listed examples.

The scanner body 200 may include a scanner-side communication part 240 for transmitting and receiving image data and/or a control signal. The scanner-side communication part 240 may perform wireless communication with an external electronic apparatus (e.g., 500 in FIG. 1) through multiple communication channels. Here, the communication channels may refer to a communication network for transmitting and receiving a radio signal through a predetermined frequency band. Specifically, the communication channels may correspond to a communication network through which a radio signal of a frequency band defined according to a predetermined radio communication standard is transmitted and received. Here, the radio communication standard may include a communication standard such as WiGig (Wireless Gigabit), Bluetooth, Wi-Fi, Bluetooth Low Energy (BLE), NFC/RFID, Wi-Fi Direct, UWB, ZIGBEE, or the like. Furthermore, the communication network conforming to a predetermined frequency band, or a predetermined communication standard may be referred to as a communication channel.

The scanner body 200 may further include a scanner-side storage part 250. The scanner-side storage part 250 may store information of the scanner body 200 (a scanner serial number of the scanner or the like) and information related to scanner control. Depending on cases, the scanner-side storage part 250 may store image data generated by an operation of the camera 232. At least one of known recording apparatuses such as a ROM, a RAM, an SSD, an HDD, or a flash memory may be used as the scanner-side storage part 250, and thus detailed descriptions thereof are omitted.

Hereinafter, a process of performing calibration of the scanner body 200 according to detection of attachment and detachment of the tool 100 will be described.

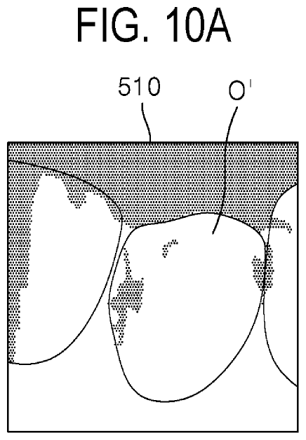

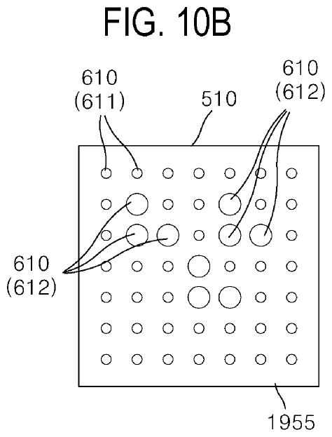

FIG. 10A is an example of image data acquired when a scanning tool 101 is attached to an end of a scanner body 200 and FIG. 10B is an example of image data acquired when a calibration tool 102 is attached to an end of the scanner body 200.

Referring to FIGS. 3, 8, 9, 10A, and 10B, when the tool 100 is attached to or removed (detached) from one end of the scanner body 200, the attachment/detachment detection sensor 220 may detect attachment or detachment of the tool 100. In this case, when the tool 100 is attached to one end of the scanner body 200, the attachment/detachment detection sensor 220 may measure and acquire a distance d between the inner surface of the tool 100 and the attachment/detachment detection sensor 220. In case that the distance d is less than or equal to a predetermined attachment/detachment threshold, it may be determined that the tool 100 is coupled to one end of the scanner body 200. For example, the attachment/detachment threshold may be about 5 mm.

When attachment/detachment of the tool 100 is detected by the attachment/detachment detection sensor 220, the control unit may control the projector 231, and the projector 231 may emit output light toward the tool 100. In this case, the output light may correspond to light in a state in which the above-described pattern generation part 2312 is not applied. Depending on a type of the tool 100 coupled to the scanner body 200, output light emitted from the projector 231 may reach a surface of the object O and/or a surface of the pattern plate 1955. In case that the tool 100 coupled to the scanner body 200 corresponds to the scanning tool 101, the output light emitted from the projector 231 may reach the surface of the object O and reflection light reflected from the surface of the object O may be received in a camera 2321 by an optical path change member 1103. The received light may be converted into image data and analyzed. In particular, when attachment of the tool 100 is identified by the attachment/detachment detection sensor 220, the control unit may control the projector 231 to emit output light toward the tool 100. The output light may illuminate the inside of the tool 100, and the camera 232 may acquire a clear image of the inside of the tool 100 due to the output light. That is, when the tool 100 is attached to the scanner body 200 and the output light of the projector 231 is emitted, the control unit may distinguish and determine whether the type of the tool 100 is the scanning tool 101 or the calibration tool 102, based on the image data acquired by the camera 232. By way of example, when it is determined that the image data acquired by the camera 232 indicates the shape of the pattern plate 1955 embedded in the calibration tool 102, the control unit may distinguish and determine that the tool 100 attached to the scanner body 200 is the calibration tool 102.

As another example, when it is determined that the image data acquired by the camera 232 does not indicate the shape of the pattern plate 1955, the control unit may distinguish and determine that the tool 100 attached to the scanner body 200 is a tool (e.g., the aforementioned scanning tool 101, and a protection tool (not shown) for protecting components of the scanner body 200) other than the calibration tool 102. In other words, the control unit may distinguish the calibration tool 102 from other tools 100 by detecting (or determining) the shape of the pattern plate 1955.

Further, the control unit may distinguish and determine whether the type of the tool 100 is an auto-calibration tool or a manual calibration tool, based on image data acquired by the camera 232.

The control unit may determine the type of the tool 100 attached to the scanner body 200 based on the image data acquired by the camera 232, and in particular, the control unit may perform a calibration operation when determining the tool 100 as the calibration tool 102.

According to the drawing in FIG. 10A, the image data acquired by the scanner body 200 having the scanning tool 101 attached thereto is displayed on the display unit 510. The image data acquired by the scanner body 200 having the scanning tool 101 attached thereto may represent the object image O.

According to the drawing in FIG. 10B, the image data acquired by the scanner body 200 having the calibration tool 102 attached thereto may represent the pattern plate 1955. The control unit may distinguish and detect whether the tool 100 attached to one end of the scanner body 200 is the scanning tool 101 or the calibration tool 102, based on the shape of the pattern plate 1955.

For example, when the shape of the image data is determined to represent the pattern plate 1955 of the calibration tool 102, the control unit may determine that the type of tool 100 coupled to the attachment/detachment surface 202 of the scanner body 200 corresponds to the calibration tool 102 and automatically perform a calibration operation. In addition, when the type of the tool 100 is determined to be the calibration tool 102, the control unit may automatically execute a calibration application on the display unit 510 to perform calibration. Further, when the type of the tool 100 is determined to be the calibration tool 102, the calibration application on the display may be automatically executed and a guide message for performing calibration may be provided to the user.

As another example, when the image data is determined to represent a shape different from that of the pattern plate 1955, the control unit may determine that the type of the tool 100 coupled to the attachment/detachment surface 202 of the scanner body 200 corresponds to the scanning tool 101, and perform a standby operation for acquiring two-dimensional image data and a three-dimensional model of the object O by an operation of the optical part 230 or perform a scanning operation.

Hereinafter, a method for the control unit of distinguishing the calibration tool 102 including the pattern plate 1955 from the scanning tool 101 will be described.

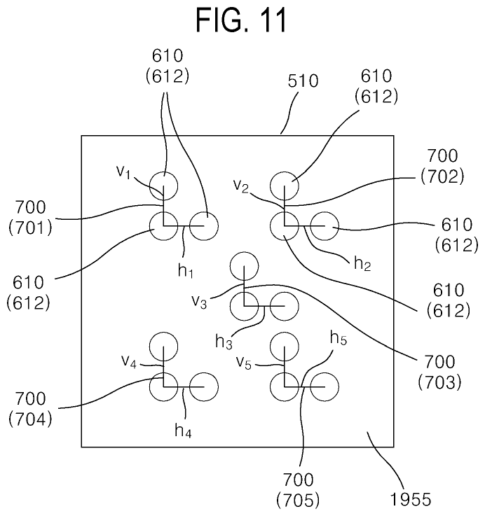

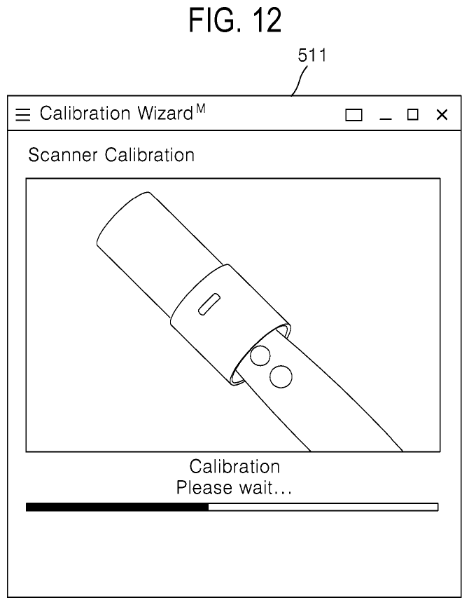

FIG. 11 is a view illustrating an example of identification targets 612 formed on the pattern plate 1955 of the calibration tool 102 and a sign 700 formed by the identification targets 612, and FIG. 12 is a view illustrating an example showing a process of performing calibration of the scanner body 200 by using the calibration tool 102. In FIG. 11, representation of the calibration targets 611 is omitted for convenience of explanation.

Referring to FIGS. 10B and 11, the pattern plate 1955 may include multiple targets 610. The multiple targets may be arranged according to a predetermined rule, at least some of the multiple targets may be used for calibration of the scanner body 200, and the others of the multiple targets may be used by the control unit to detect the type of the tool 100 attached to one end of the scanner body 200.

By way of example, the multiple targets 610 may include at least one calibration target 611 and multiple identification targets 612. The calibration target 611 may be used for calibration of the scanner body 200, and the calibration target 611 may be arranged in a predetermined array form. A configuration of the scanner body 200 is adjusted through the calibration target 611, and the scanner body 200 may be maintained to acquire a precise three-dimensional model of the object.

In addition, the identification targets 612 may be formed between calibration targets 611, and the multiple identification targets 612 may be arranged. The identification targets 612 may be spaced apart from the calibration target 611 and may have a shape different from that of the calibration target 611. For example, each of the identification targets 612 may have a circular shape having a larger diameter than the calibration target 611. However, the shape of the identification targets 612 is not limited to the described example, and each of the identification targets 612 may have a shape distinguished from that of the calibration target 611. Through the image data in which the identification target 612 is represented, the control unit may distinguish the type of the tool 100 attached to one end of the scanner body 200.

At least some of the multiple identification targets 612 may form a predetermined sign 700. As shown in FIG. 11, the predetermined sign 700 may be configured by connecting at least some of adjacent identification targets 612. For example, the sign 700 having a predetermined shape may be formed by connecting three identification targets 612. The sign 700 may have an “L” shape.

Further, for example, the sign 700 on the pattern plate 1955 may include a first sign 701, a second sign 702, a third sign 703, a fourth sign 704, and a fifth sign 705, and each of the signs 701, 702, 703, 704, and 705 may have first lengths h1, h2, h3, h4, and h5, respectively, and second lengths v1, v2, v3, v4, and v5, respectively. In this case, the first length may indicate a horizontal length between adjacent identification targets 612, and the second length may indicate a vertical length between adjacent identification targets 612. Here, the first length and the second length may be acquired based on the center of each of the identification targets 612, but are not limited to the presented reference.

The control unit may detect the calibration target 611, detect the identification targets 612, or detect the sign 700 formed by the identification targets 612 from the image data acquired by an operation of the camera, or detect the first length and/or the second length of the sign 700, so as to determine whether the tool 100 attached to the scanner body 200 is the calibration tool 102 including the pattern plate 1955. As such, by detecting the type of the tool 100 through at least one of the calibration target 611, the identification targets 612, the mark 700, or the dimension of the mark 700 from the image data, the control unit may quickly and accurately distinguish and determine the tool 100, thus having an advantage in that the calibration of the scanner body 200 may be easily performed accordingly.

Referring to FIG. 12, a calibration application screen 511 may be displayed on a display unit (e.g., 500 in FIG. 1). That is, the control unit may perform a calibration operation when determining that the tool 100 coupled to the scanner body 200 corresponds to the calibration tool 102.

Depending on cases, the control unit may automatically execute a calibration application when determining that the tool 100 coupled to the scanner body 200 corresponds to the calibration tool 102. That is, when determining that the tool 100 corresponds to the calibration tool 102, the control unit may execute the calibration application to be visually displayed on the display unit 510, and according to a calibration function built into the calibration application, may perform calibration of the scanner body 200. Accordingly, there is an advantage in that inconvenience is minimized for the user by omitting a separate application execution process and calibrating the scanner body 200 automatically.

Hereinafter, an embodiment in which the type of the tool 100 attached to one end of the scanner body 200 is identified and determined by the attachment/detachment detection sensor 220 will be described.

In an embodiment disclosed in the present disclosure, the distance d between the attachment/detachment detection sensor and the tool 100 may vary depending on the type of the tool 100. By way of example, the scanning tool 101 may have a protrusion (not shown) structure protruding inward toward the attachment/detachment detection sensor.

In this case, the control unit may determine the type of the tool 100 based on the distance d between the attachment/detachment detection sensor 220 and the tool 100 when the tool 100 is attached to the scanner body 200. The control unit may determine that the tool 100 corresponds to the scanning tool 101 when the distance d is equal to or less than a first distance threshold, and determine that the tool 100 corresponds to the calibration tool 102 when the distance d is less than or equal to a second distance threshold. The first distance threshold may be set to be smaller than the second distance threshold. When determining that the tool 100 corresponds to the calibration tool 102, the control unit may perform a calibration operation of the scanner body 200. As such, by distinguishing the type of the tool 100 based on the distance d acquired by measurement of the attachment/detachment detection sensor 220, there is an advantage in that the tool 100 may be quickly distinguished and quick calibration is possible when the calibration tool 102 is attached to the scanner body 200.

According to an embodiment disclosed in the present disclosure, an identifier such as a barcode or QR code may be marked on the tool 100, and the control unit may identify and determine the type of the tool 100 by recognizing the identifier marked on the tool 100 by using the camera 232 or a separate recognition unit (not shown).

Hereinafter, a process of detecting an auto-calibration tool and a manual calibration tool will be described.

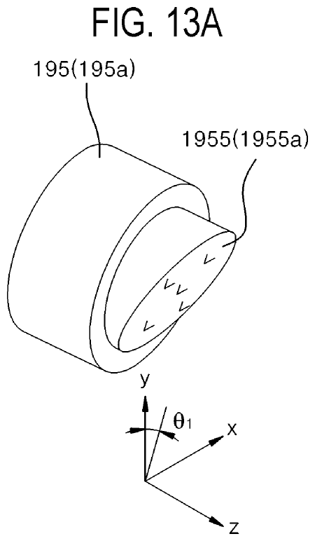

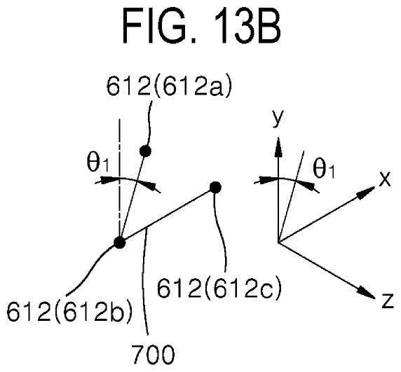

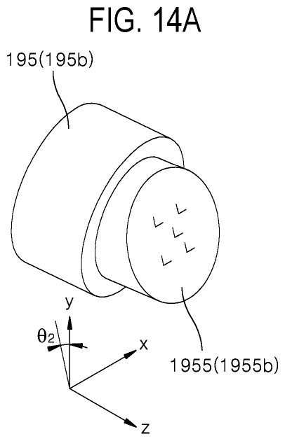

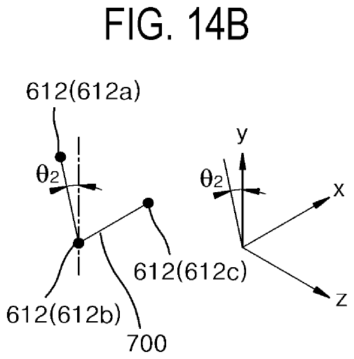

FIG. 13A is an example illustrating an auto-calibration tool, and FIG. 13B is an example illustrating three-dimensional information of identification targets 612 which form a sign 700 of the auto-calibration tool. FIG. 14A is an example illustrating a manual calibration tool, and FIG. 14B is an example illustrating three-dimensional information of identification targets 612 which form a sign 700 of the manual calibration tool.

Referring to FIGS. 13A and 13B, the calibration tool 102 may include an auto-calibration tool. A mounting block 195 or 195a and a pattern plate 1955 or 1955a applied to the auto-calibration tool may be formed to be tilted in a first rotation direction. For example, the pattern plate 1955 or 1955a of the auto-calibration tool may be formed to be tilted by θ1 clockwise with respect to the y-axis on the coordinate space having an axis direction of z for the calibration tool 102. When the pattern plate 1955 or 1955a is formed to be tilted by θ1, image data acquired by the camera 2321 built in the scanner body 200 may also be tilted and acquired. Here, at least one of the first length or the second length of the sign 700 formed on the pattern plate 1955 or 1955a may be changed.

Referring to FIGS. 14A and 14B, the calibration tool 102 may include a manual calibration tool. A mounting block 195 or 195b and a pattern plate 1955 or 1955b applied to the manual calibration tool may be formed to be tilted in a second rotation direction. Here, the second rotation direction may be a rotation direction opposite to the first rotation direction. For example, the pattern plate 1955 or 1955b of the manual calibration tool may be formed to be tilted by θ2 counterclockwise with respect to the y-axis on the coordinate space having an axis direction of z for the calibration tool 102. When the pattern plate 1955 or 1955b is formed to be tilted by θ2, image data acquired by the camera 2321 built in the scanner body 200 may also be tilted and acquired. Here, at least one of the first length and the second length of the sign 700 formed on the pattern plate 1955 or 1955b may be changed.

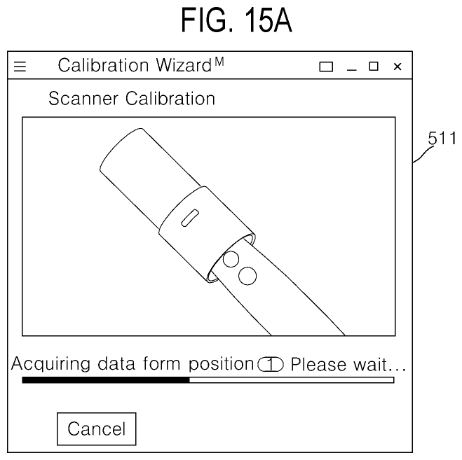

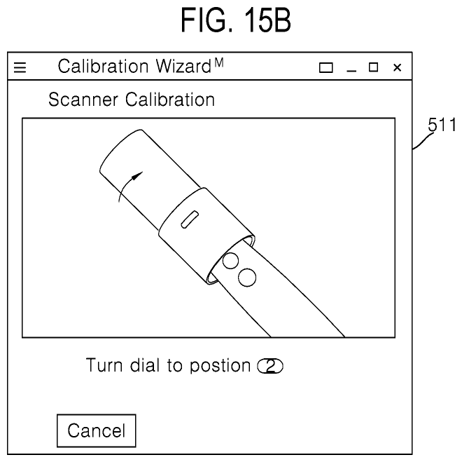

FIGS. 15A and 15B are examples illustrating a process of performing calibration of a scanner body 200 by using a manual calibration tool. More specifically, FIG. 15A is for describing a process in which calibration of the scanner body 200 is performed when the pattern plate of the manual calibration tool is placed in a first location, and FIG. 15B is for describing a process of displaying a predetermined guide on a calibration application screen 511 to dispose the pattern plate of the manual calibration tool to a second location.

Referring to FIGS. 13A to 15B, the control unit may distinguish a type of a calibration tool 102 on the basis of image data acquired by photographing the pattern plate 1955. Specifically, the control unit determines the type of the calibration tool 102 as either a manual calibration tool or an auto-calibration tool based on the shape of the pattern plate 1955 included in the calibration tool 102, and may perform a calibration operation corresponding to the type of the tool 102.

By way of example, the control unit may distinguish whether the type of the calibration tool 102 is an auto-calibration tool or a manual calibration tool based on at least one of the first length or the second length of the sign 700. When the control unit distinguishes the type of the calibration tool 102, the control unit may apply a control signal corresponding to the type of the calibration tool 102 to the scanner body 200. For example, when the calibration tool 102 is an auto-calibration tool, the control unit may automatically perform calibration of the scanner body 200 by controlling at least one selected from the group of the scanner body 200 and the auto-calibration tool. For another example, when the calibration tool 102 is a manual calibration tool, the control unit may control a guide message for guiding the user to operate the manual calibration tool to be displayed on the application screen 511 on the display device 510, and the user may operate the manual calibration tool according to the guide message displayed on the application screen 511.

Referring to FIG. 15A as an example, a guide to place a dial of the calibration tool 102 at a first location may be displayed on the calibration application screen 511 under control of the control unit. When the user places the dial of the calibration tool 102 at the first location, a calibration process of the scanner body 200 at the first location is performed. Thereafter, referring to FIG. 15B as an example, a guide to place a dial of the calibration tool 102 at a second location may be displayed on the calibration application screen 511 under control of the control unit. When the user places the dial of the calibration tool 102 at the second location to move and/or rotate the pattern plate, a calibration process of the scanner body 200 at the second location is performed. The manual calibration tool may move or rotate the pattern plate to multiple locations (or angles) through the dial, and thus the calibration of the scanner body 200 may be performed.

As such, since the operation of the scanner corresponding to each tool is performed when one of the auto-calibration tool or the manual calibration tool is attached, there is an advantage in that the user may conveniently and quickly perform the calibration of the scanner body 200.

Hereinafter, another method for distinguishing an auto-calibration tool and a manual calibration tool will be described.

In the image processing device 1 according to an embodiment disclosed in the present disclosure, the scanner body 200 may include at least one camera 232, and multiple cameras 232 may be provided. Here, at least two pieces of image data may be acquired by the multiple cameras 232a and 232b. Depth information may be acquired based on the image data acquired by the multiple cameras 232a and 232b, that is, at least a portion of three-dimensional information of the calibration tool 102 may be acquired. For example, it is assumed that the identification targets 612 forming the sign 700 include a first identification target 612a, a second identification target 612b, and a third identification target 612c. In this case, tilting of the pattern plate 1955 causes a three-dimensional location of the first identification target 612a disposed on the auto-calibration tool to protrude by a predetermined thickness in the positive z-axis direction compared to those of the second identification target 612b and the third identification target 612c. In another example, tilting of the pattern plate 1955 causes a three-dimensional location of the first identification target 612a disposed on the manual calibration tool to be recessed by a predetermined depth in the negative z-axis direction compared to those of the second identification target 612b and the third identification target 612c.

Therefore, the user may distinguish the type of the calibration tool 102 based on the three-dimensional locational relationship of the identification targets 612, and the control unit may apply a control signal corresponding to the type of the calibration tool 102.

The foregoing description has been made that the pattern plate 1955 or 1955a of the auto-calibration tool is formed to be tilted clockwise with respect to the y-axis and the pattern plate 1955 or 1955b of the manual calibration tool is tilted counterclockwise with respect to the y-axis. However, the tilting directions of the auto-calibration tool and the manual calibration tool are not limited to the above, and it is also possible that the pattern plate 1955 or 1955a of the auto-calibration tool is tilted counterclockwise with respect to the y-axis and the pattern plate 1955 or 1955b of the manual calibration tool is formed to be tilted clockwise with respect to the y-axis.

The method for the control unit to distinguish the auto-calibration tool and the manual calibration tool is not limited to the above description. For example, when the auto-calibration tool and the manual calibration tool have different sign shapes on the pattern plate 1955, the control unit may distinguish and determine whether the calibration tool 102 coupled to the scanner body 200 is the auto-calibration tool or the manual calibration tool, based on the different sign shapes.

As another example, the control unit may distinguish and determine whether the calibration tool 102 coupled to the scanner body 200 is the auto-calibration tool or the manual calibration tool, based on the shape of the identification target (inclined shape of the identification target, size of the identification target, etc.) formed on the pattern plate 9155 of each of the auto-calibration tool or the manual calibration tool.

In the foregoing, the process of distinguishing/determining the scanning tool 101 and the calibration tool 102 by the control unit and/or the process of distinguishing/determining the automatic calibration tool and the manual calibration tool by the control unit may be performed through analysis of two-dimensional image data using artificial intelligence without acquiring three-dimensional information of the identification target.

Hereinafter, an image processing method using the image processing apparatus according to an embodiment of the present disclosure will be described. In describing the image processing method, descriptions of overlapping contents of the image processing apparatus according to an embodiment disclosed in the present disclosure will be briefly mentioned or omitted.

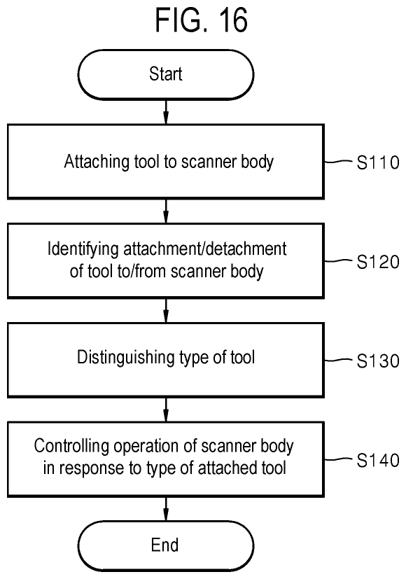

FIG. 16 is an exemplary flowchart illustrating an image processing method using an image processing apparatus according to the present disclosure.

Referring to FIG. 16, the image processing method using the image processing apparatus according to an embodiment disclosed in the present disclosure includes a tool attachment operation S110, a tool attachment/detachment identification operation S120, a tool distinguishing operation S130, and scanner control operation S140.

In the tool attachment operation S110, the user may mount a predetermined tool on the scanner body. For example, in the tool attachment operation S110, the tool attached to one end of the scanner body may include a calibration tool for calibration of the scanner body. The calibration tool may be formed in a form of a dark room with one side closed to block exit of light from the outside. As another example, the tool attached (coupled) to one end of the scanner body in the tool attachment operation S110 may include a scanning tool formed in a shape with one side open.

Meanwhile, in the tool attachment operation S110, the user may detach a tool previously attached and then mount a different tool from the detached tool. As an example, the user may detach the scanning tool attached to one end of the scanner body and mount the calibration tool on one end of the scanner body. By way of another example, the user may detach the calibration tool attached to one end of the scanner body and mount the scanning tool on one end of the scanner body.

In the tool attachment/detachment identification operation S120, the control unit may determine attachment/detachment of the tool to/from the scanner body. For example, the control unit may determine attachment/detachment of the tool to the scanner body based on a measurement of an attachment/detachment detection sensor which is formed on at least a portion of the scanner body and identifies detachment/attachment of the tool. For example, the attachment/detachment detection sensor may correspond to a proximity sensor, and the attachment/detachment detection sensor may measure and acquire a distance between the attachment/detachment detection sensor and a tool coupled to the scanner body.

When attachment/detachment of the tool to/from the scanner body is identified in the tool attachment/detachment identification operation S120, a tool distinguishing operation S130 may be performed. Here, the control unit may distinguish the type of the tool. A process for the control unit to distinguish the type of the tool in the tool distinguishing operation S130 will be described below.

After the control unit distinguishes the type of the tool attached to the scanner body by the tool distinguishing operation S130, the scanner control operation S140 may be performed. In the scanner control operation S140, the control unit may control an operation of the scanner body in response to the type of the distinguished tool. As an example, when the tool attached to the scanner body is distinguished as the calibration tool in the tool distinguishing operation S130, the control unit may control the calibration operation of the scanner body. As another example, when the tool attached to the scanner body is distinguished as the scanning tool in the tool distinguishing operation S130, the control unit may control the scan operation of the scanner body.

Hereinafter, an example of the tool distinguishing operation S120 of the image processing method using the image processing apparatus according to an embodiment of the present disclosure will be described.

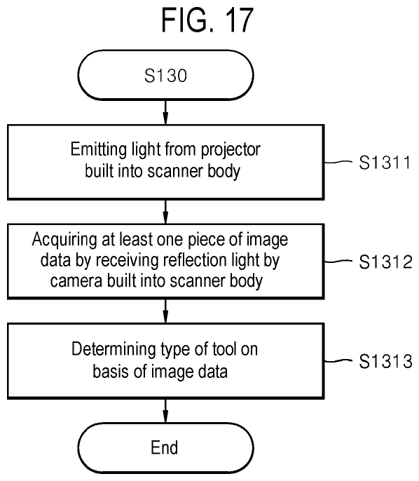

FIG. 17 is an example illustrating the tool distinguishing operation S130 of the image processing method using the image processing apparatus according to the present disclosure.

Referring to FIGS. 16 and 17, in the tool distinguishing operation S130, the control unit may distinguish the type of the tool attached to one end of the scanner body by using the optical unit built into the scanner body. The optical part may include a projector for emitting predetermined output light toward the tool and at least one camera for receiving reflection light generated by reflection of the output light. As an example, when the output light reaches an object (e.g., inside the patient's oral cavity) disposed in the outside or a pattern plate embedded in a calibration tool, the output light is reflected from a surface (or inside) of the object or pattern plate to generate reflection light, and the reflection light may be guided to one end of the scanner body and received through a camera lens, which is a component of the camera. The received reflection light may be analyzed by an imaging board (more specifically, an image sensor included in the imaging board) electrically connected to the camera, and image data representing the object or pattern plate may be generated.

When the attachment of the tool to the scanner body is identified in the tool attachment/detachment identification operation S120, the control unit controls the projector to perform a light emission operation S1311 of emitting predetermined output light from the inside of the scanner body toward the tool side. For example, the output light may correspond to light in a visible light wavelength region, but is not necessarily limited to the corresponding wavelength region. If necessary, the output light may correspond to structured light generated through a pattern generation part (e.g., a pattern mask and/or DMD) included in the projector.

When output light is emitted in the light emission operation S1311, an image data acquisition operation S1312 may be performed. In the image data acquisition operation S1312, the camera built in the scanner body may acquire at least one piece of image data by receiving the reflection light generated by reflection of output light. The image data may correspond to at least one of two-dimensional data and three-dimensional data. For example, when the camera module includes two or more cameras (e.g., an L camera and an R camera), image data by each of the L camera and the R camera may be acquired.

When image data is acquired, a tool determination operation S1313 may be performed. In the tool determination operation S1313, the control unit may determine the type of the tool based on the acquired image data. As shown in FIG. 10A, if the acquired image data indicates a shape of the object, the control unit may determine the type of the tool attached to the scanner body as a scanning tool. In addition, if the acquired image data indicates a shape of the pattern plate, the control unit may determine the type of the tool attached to the scanner body as a calibration tool. That is, the control unit may determine the type of the tool by determining the shape of the pattern plate embedded in the calibration tool through the image data. In this case, the shape of the pattern plate identified and determined by the control unit through the image data may correspond to multiple targets included in the pattern plate.

The multiple targets may include at least one calibration target and multiple identification targets. The calibration target may be used for calibration of the scanner body. An identification target may be spaced apart from the calibration target, may have a shape different from that of the calibration target, and may be used to distinguish a type of a calibration tool. Further, in the tool determination operation S1313, the control unit may determine the type of the tool based on a predetermined sign formed by at least a portion of multiple identification targets.

By way of example, in the tool determination operation S1313, the control unit may determine a tool attached to the scanner body as a calibration tool when a calibration target and/or an identification target exist in the acquired image data. As another example, in the tool determination operation S1313, the control unit may determine a tool attached to the scanner body as a calibration tool when a sign exists in the acquired image data. As yet another example, in the tool determination operation S1313, the control unit may determine a tool attached to the scanner body as a calibration tool based on the shape and size of a sign or the shape and size of an identification target existing in the acquired image data. However, the elements listed in the embodiments disclosed in the present disclosure to describe a reference for determining the type of the tool are exemplary, and other references may be applied as needed.

Hereinafter, another example of the tool distinguishing operation S130 for distinguishing a tool attached to the scanner body will be described.

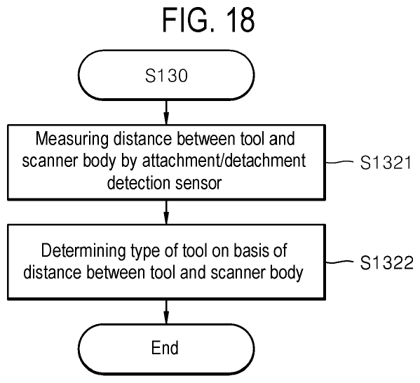

FIG. 18 is another example illustrating the tool distinguishing operation S130 of the image processing method using the image processing apparatus according to the present disclosure.

Referring to FIGS. 16 and 18, in the tool distinguishing operation S130l, the control unit may detect the type of the tool based on a distance between the attachment/detachment detection sensor and the tool when the tool is attached to the scanner body. The control unit may distinguish and determine that the tool corresponds to the scanning tool when the distance is equal to or less than a first distance threshold, and distinguish and determine that the tool corresponds to the calibration tool when the distance is less than or equal to a second distance threshold. The first distance threshold may be set to be smaller than the second distance threshold, but the first distance threshold may be set to be higher than the second distance threshold, if necessary.

In the tool distinguishing operation S130 performed according to another example of the present disclosure, the distance measurement operation S1321 may be performed. In the distance measurement operation S1321, the attachment/detachment detection sensor formed on at least a portion of the scanner body may measure the distance between the tool and the scanner body when the tool is attached to one end of the scanner body. The distance may correspond to a vertical distance from the attachment/detachment detection sensor to the inner surface of the tool, but is not limited thereto.

Thereafter, the tool determination operation S1322 may be performed. In the tool determination operation S1322, the control unit detects and determines the type of the tool based on the distance between the tool and the scanner body acquired by the attachment/detachment detection sensor. The process of determining the type of the tool based on the distance between the tool and the scanner body is the same as the above description, and thus a detailed description thereof will be omitted.

Hereinafter, a detailed operation of the scanner control operation S140 will be described.

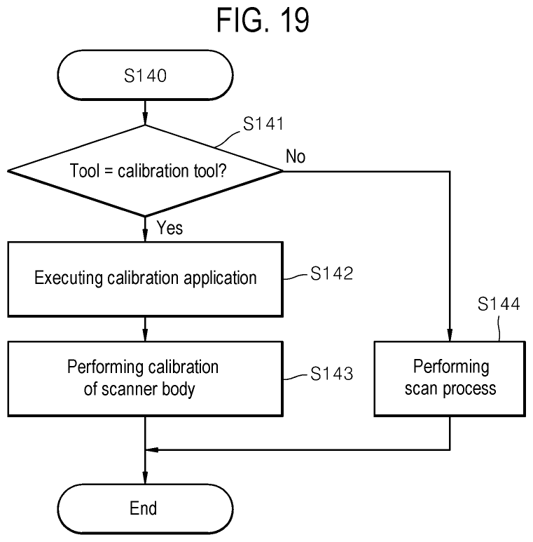

FIG. 19 is an example illustrating the scanner control operation S140 of the image processing method using the image processing apparatus according to the present disclosure.

Referring to FIGS. 16 and 19, a tool type identification operation S141 may be performed. In the tool type identification operation S141, the control unit may identify whether the tool determined in the tool distinguishing operation S130 is a calibration tool.

After the tool-type identification operation S141 is performed, a calibration application execution operation S142 may be performed. More specifically, when the control unit detects and distinguishes the tool as a calibration tool, the control unit may automatically execute a calibration application. Accordingly, the calibration of the scanner body may be performed by the executed calibration application.

When the control unit distinguishes the type of the tool as a calibration tool, a calibration performance operation S143 may be performed. The control unit may control the scanner body to perform a calibration operation in the scanner control operation S140. The calibration may represent correcting a configuration of at least some (particularly, the optical part) of the components built into the scanner body.

Meanwhile, when the tool attached to the scanner body is identified as a scanning tool in the tool type identification operation S141, a scanning operation S144 may be performed. It may be determined that the scanning tool attached to the scanner body is attached by the user to scan the object, and the control unit controls the optical part so that the scanner body may acquire a three-dimensional model and/or an image representing the object.

Hereinafter, a detailed operation of the calibration performance operation S143 will be described.

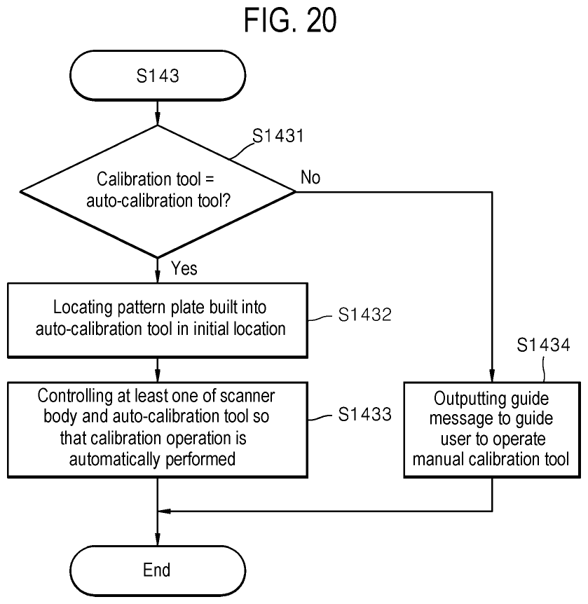

FIG. 20 is an example illustrating the calibration performance operation S143 of the image processing method using the image processing apparatus according to the present disclosure.

Referring to FIGS. 16, 19, and 20, a calibration process may be different depending on whether the calibration tool is an auto-calibration tool or a manual calibration tool. Accordingly, the calibration performance operation S143 may include a calibration tool identification operation S1431. In the calibration tool identification operation S1431, the control unit may identify a type of the calibration tool that has been distinguished, detected, and/or determined in the tool distinguishing operation S130 or the tool determination operation S1313 or S1322, which is detailed operation of the tool distinguishing operation. For example, the calibration tool may correspond to an auto-calibration tool that automatically moves and/or rotates the pattern plate, or a manual calibration tool that allows the user to move and/or rotate the pattern plate using a dial lever.

Meanwhile, the auto-calibration tool and the manual calibration tool may be distinguished according to the tool determination operation S1313 of determining the type of the tool based on image data. As an example, the pattern plates included in the auto-calibration tool and the manual calibration tool may be formed to be tilted at different angles. The auto-calibration tool and the manual calibration tool may be distinguished based on at least one of a difference in sizes of identification targets that appear when the pattern plate is formed to be tilted at different angles, or a difference in shapes of predetermined signs formed by at least some of the identification targets (e.g., at least one of the first length and the second length of the sign, or the shape and size or the entire sign itself).

Further, as described above, when acquiring three-dimensional information of identification targets included in the pattern plate, the auto-calibration tool and the manual calibration tool may be distinguished based on three-dimensional locations of the identification targets.

When the control unit identifies the tool as the manual calibration tool in the tool distinguishing operation S130 and the control unit identifies the calibration tool as the manual calibration tool in the calibration tool identification operation S1431, a manual calibration performance operation S1434 may be performed. In the manual calibration performance operation S1434 of the scan operation control operation S140, the control unit may generate an instruction for guiding the user to operate the manual calibration tool. For example, the control unit may control the calibration application screen 511 to be displayed on the display unit 510, and as shown in FIG. 15B, the user may operate the manual calibration tool to induce calibration of the scanner body to proceed.

On the other hand, when the control unit identifies the tool as the auto-calibration tool in the tool distinguishing operation S130 and the control unit identifies the calibration tool as the auto-calibration tool in the calibration tool identification operation S1431, an auto-calibration performance operation S1433 may be performed. In the auto-calibration performance operation S1433 of the scan operation control operation S140, the control unit may control at least one selected from the group of the scanner body and the auto-calibration tool so that the scanner body automatically performs a calibration operation. In this case, the auto-calibration tool may operate so that the pattern plate is automatically moved and/or rotated according to the calibration process of the scanner body by the above-described pattern moving part. Accordingly, the calibration of the scanner body may be automatically performed.

Depending on cases, the pattern plate built into the auto-calibration tool may not be placed in an initial location thereof. Therefore, the calibration performance operation S143 of the image processing method according to an embodiment disclosed in the present disclosure may further include a pattern plate location operation S1432. In the pattern plate location operation S1432, the control unit may control the pattern plate built into the auto-calibration tool to be located in the initial location before the calibration operation (more specifically, the auto-calibration operation S1433) of the scanner control operation S140 is performed.

For example, initial location information of the pattern plate may be stored in the external electronic device and/or the scanner body. Therefore, in case that the auto-calibration tool is attached to the scanner body, the control unit may apply a control signal to the auto-calibration tool so as to control a location of the pattern plate to be the initial location thereof. Accordingly, there is an advantage in that accurate calibration of the scanner body may be performed through the auto-calibration tool.

The image processing method using the image processing apparatus according to various embodiments of the present disclosure may be implemented in a form of program command to be executed through various computer means and recorded on a computer readable medium. In addition, an embodiment of the present disclosure may correspond to a computer-readable recording medium on which one or more programs including commands for executing the calibration process of the scanner body are recorded.

The computer readable medium may include program commands, data files, data structures, etc. alone or in combination. Program commands recorded on the medium may be specially designed and configured for the present disclosure or known and usable to those skilled in computer software. Examples of computer-readable recording media include magnetic media such as hard disks, floppy disks, and magnetic tapes, optical media such as CD-ROMs and DVDs, magneto-optical media such as floptical disks, and hardware apparatuses specially configured to store and execute program commands, such as a ROM, a RAM, a flash memory, and the like. Examples of program commands include high-level language code that may be executed by a computer using an interpreter, as well as machine language code produced by a compiler.

Here, a device-readable storage medium may be provided in the form of a non-transitory storage medium. In this case, the term “non-temporary” merely means that the storage medium is a tangible device and does not include a signal (e.g., electromagnetic wave), and the term does not distinguish the case where data is stored semi-permanently in the storage medium and the case where data is stored temporarily. For example, the term “non-temporary storage medium” may include a buffer in which data is temporarily stored.

According to an embodiment, the image processing method using the image processing apparatus according to various embodiments disclosed herein may be included in a computer program product, and provided. A computer program product may be traded between sellers and buyers as a commodity. The computer program product may be distributed in the form of a machine-readable storage medium (e.g., a compact disc read only memory (CD-ROM)), or distributed on-line (e.g., downloaded or uploaded) through an application store (e.g., Play Store™) or directly between two user devices (e.g., smartphones). In the case of on-line distribution, at least a portion of the computer program product (e.g., a downloadable app) may be stored, or at least temporarily generated, on a device-readable storage medium, such as a manufacturer's server, an application store's server, or the memory of a relay server.

Specifically, the image processing method using the image processing apparatus according to various embodiments disclosed herein may be implemented as a computer program product including a recording medium for storing a program to perform an operation of acquiring a multilingual sentence and an operation of acquiring vector values corresponding to each of words included in the multilingual sentence by using a multilingual translation model, converting the acquired vector values to vector values corresponding to a target language, and based on the converted vector values, acquiring a sentence including the target language.

It is to be understood that the image processing apparatus, the image processing method using the image processing apparatus, and the recording medium according to the embodiments disclosed in the present disclosure mutually share the aforementioned advantages.

The above description is merely an example of the technical ideas of the disclosure, and various modifications and variations will be apparent to those skilled in the art to which the disclosure belongs, without departing from the essential features of the disclosure.

Therefore, the embodiments disclosed in the present disclosure are intended to illustrate, not limit, the technical ideas of the present disclosure, and the scope of the technical ideas of the present disclosure is not limited by these embodiments. The scope of protection of the present disclosure should be construed in accordance with the following claims, and all technical ideas within the scope of the equivalents should be construed to be included within the scope of the present disclosure.

An embodiment disclosed in the present disclosure provides an image processing apparatus and an image processing method using the image processing apparatus for maintaining a high scanning precision of a scanner body by performing a corresponding operation of the scanner body according to a type of a tool detachably coupled to the scanner body.

### Image Processing Apparatus and Image Processing Method Using Same

### TECHNICAL FIELD

The present disclosure relates to an image processing apparatus and an image processing method using same. Specifically, the present disclosure relates to an image processing apparatus, in which an operation of a scanner body corresponding to a tool is performed by the tool attached to the scanner body, and an image processing method using same.

### BACKGROUND

Performance of optical apparatus may be sensitive to temperature and, specifically, an optical part of a three-dimensional scanner used for acquiring a three-dimensional model of a real object (in the present disclosure, referred to as “object”) may include a projector for emitting light toward the object and a camera for receiving light reflected from the object. The three-dimensional scanner (particularly, the optical part) needs to maintain high precision to acquire a three-dimensional model representing the object more accurately. Therefore, a user may need to perform error correction for the three-dimensional scanner, that is, calibration at predetermined intervals to keep the precision of the three-dimensional scanner high.

An example of a conventional process in which the user performs calibration of the three-dimensional scanner will be described. The user performs a scan program in a state in which a scanner body of the three-dimensional scanner is connected to a computing apparatus including a processor. When the scan program is performed, the user attaches a calibration tool to one end of the scanner body. Thereafter, the user selects a calibration button displayed on a user interface of the scan program to use a calibration function provided by the scan program. Although it has been described that the calibration tool is attached to one end of the scanner body before the calibration button is selected in the description above, it is not limited thereto, and the calibration tool may be attached to the scanner body before or after the calibration button is selected.

When the user selects the calibration button, a calibration application is displayed in a pop-up form and the user performs calibration of the scanner body according to an indication of the calibration application.

In the process of performing the calibration as described above, the user may recognize that the calibration of the scanner body is required, through a predetermined notification (e.g., an overdue message displayed on the scan program) after executing the scan program and moving to a clinic chair to scan a patient's mouth. In this case, the user may need to move to the computing apparatus side from the chair again to perform the calibration and this process may cause inconvenience to the user. Further, after performing the calibration of the scanner body, in order to provide a hygienic scanning environment to the patient, the user had the inconvenience of having to change the gloves that had been worn or performing additional disinfection before scanning the patient's mouth.

### SUMMARY

To achieve the purpose of the present disclosure, an embodiment disclosed in the present disclosure provides an image processing apparatus in which a tool, which can be attached or detached to one end of a scanner body, is attached to the scanner body to perform an operation corresponding to a type of the attached tool, and an image processing method using same.

In addition, an embodiment disclosed in the present disclosure provides the image processing apparatus which may identify whether a tool is attached or detached through an attachment/detachment detection sensor provided to a scanner body, and the image processing method using same.

In addition, an embodiment disclosed in the present disclosure provides the image processing apparatus which acquires image data through an operation of an optical part built in a scanner body and distinguishes a type of tool based on the image data, and the image processing method using same.

In addition, an embodiment disclosed in the present disclosure provides the image processing apparatus which distinguishes a type of tool based on a distance between the tool and an attachment/detachment detection sensor which is provided to the scanner body and acquires the distance, and the image processing method using same.

In addition, an embodiment disclosed in the present disclosure provides the image processing apparatus which automatically performs a calibration application without a separate operation of a user when a calibration tool is determined to be attached, and the image processing method using same.

In addition, an embodiment disclosed in the present disclosure provides the image processing apparatus which performs a corresponding calibration process depending on a type of calibration tool attached to the scanner body, and the image processing method using same.

The technical problems solved by the present disclosure are not limited to the above technical problems and those skilled in the art will more clearly understand other technical problems not described above from the following description.

To achieve the objective as previously stated, the image processing device according to the disclosed embodiment of the present disclosure comprises, a tool detachably coupled to one end of a scanner body, the scanner body having one end to which the tool is coupled, and a control unit configured to control an operation of the scanner body in response to the tool.

In one embodiment, the tool may comprise a calibration tool formed into a shape with one side closed and configured to calibrate the scanner body, and a scanning tool formed into a shape with one side open.

In one embodiment, the scanner body may comprise, an attachment/detachment detection sensor formed on at least a portion of the scanner body and configured to identify whether the tool is attached to or detached from an attachment/detachment surface which couples the tool to the scanner body, a projector configured to emit a predetermined output light toward the tool, and at least one camera configured to receive reflection light generated by reflection of the output light and acquire at least one piece of image data from the reflection light.

In one embodiment, the projector may be configured to, when attachment or detachment of the tool is identified by the attachment/detachment detection sensor, emit the output light.

In one embodiment, the control unit may be configured to determine a type of the tool attached to the scanner body based on the image data acquired by the camera, and when the tool is determined as a calibration tool, perform a calibration operation.

In one embodiment, the control unit may be configured to, when a shape of the image data is determined to represent a pattern plate of the calibration tool, determine the type of the tool as the calibration tool and perform a calibration operation.

In one embodiment, the control unit may be configured to determine a type of the calibration tool as either a manual calibration tool or an auto-calibration tool based on the shape of the pattern plate, and perform a calibration operation corresponding to the type of the calibration tool.

In one embodiment, the control unit may be configured to, when the tool is attached to the scanner body, determine a type of the tool based on a distance between the attachment/detachment detection sensor and the tool, and when the tool is determined as a calibration tool, perform a calibration operation.

In one embodiment, the control unit may be configured to, when the tool is determined as a calibration tool, automatically execute a calibration application to be displayed on a display unit.

In one embodiment, the control unit may be configured to, when the tool is determined as a manual calibration tool among the calibration tool, control a guide message to be output on the display unit to guide a user to operate the manual calibration tool.

In one embodiment, the calibration tool may further comprise a pattern plate configured to be tiltable within a predetermined angle range based on a longitudinal direction of the scanner body, and wherein the pattern plate may comprise multiple targets.

In one embodiment, the multiple targets may comprise at least one calibration target for calibration of the scanner body, and multiple identification targets formed to be spaced apart from the calibration target, having a shape different from that of the calibration target, and used to identify a type of the calibration tool.

The image processing method using an image processing apparatus according to the embodiment disclosed in the present disclosure comprises a tool attachment operation in which a user attaches a predetermined tool to a scanner body, a tool attachment/detachment identification operation in which a control unit determines attachment/detachment of the tool to/from the scanner body, a tool distinguishing operation in which the control unit distinguishes a type of the tool, and a scanner control operation in which the control unit controls an operation of the scanner body in response to the type of the tool.

In one embodiment, in the tool attachment/detachment identification operation, the attachment/detachment of the tool may be identified by an attachment/detachment detection sensor formed on an attachment/detachment surface which is formed on at least a portion of the scanner body and configured to couple the tool to the scanner body.

In one embodiment, the tool distinguishing operation may comprise a light emission operation of emitting, when the attachment/detachment of the tool is identified in the tool attachment/detachment identification operation, predetermined output light from a projector built into the scanner body, an image data acquisition operation in which at least one camera built into the scanner body receives reflection light generated by reflection of the output light and acquires at least one piece of image data, and a tool determination operation of determining a type of the tool based on the image data.

In one embodiment, in the tool determination operation, the control unit may determine the type of the tool by determining a shape of a pattern plate built into a calibration tool through the image data.

In one embodiment, the control unit may be configured to, when the tool is attached to the scanner body, detect a type of the tool based on a distance between the attachment/detachment detection sensor and the tool.

In one embodiment, the method may further comprise a calibration application execution operation in which the control unit is configured to, when the control unit determines the tool as a calibration tool, automatically execute a calibration application to be displayed on a display unit.

In one embodiment, the control unit may be configured to, when the control unit distinguishes a type of the tool as a manual calibration tool among the calibration tool, control a guide message to be output on a display unit to guide the user to operate the manual calibration tool in the scanner control operation.

In one embodiment, the control unit may be configured to, when the control unit distinguishes a type of the tool as an auto-calibration tool among the calibration tool, control at least one selected from the group of the scanner body and the auto-calibration tool so that a calibration operation is automatically performed in the scanner control operation.

By using the image processing apparatus and the image processing method using the image processing apparatus according to an embodiment disclosed in the present disclosure, the user may have an advantage of minimizing inconvenience in calibrating the scanner body. Further, since the attachment/detachment of the tool is identified by the attachment/detachment detection sensor of the scanner body and the type (e.g., a scanning tool and a calibration tool, or an auto-calibration tool and a manual calibration tool) of tool is determined based on the image data acquired by a camera, there is an advantage in that the type of tool may be quickly detected and an operation (e.g., calibration of the scanner body) of the scanner matching the type of tool may be performed without a separate operating process.

Further, since the type of tool is determined based on a distance between the tool and the attachment/detachment detection sensor, which is measured by the attachment/detachment detection sensor of the scanner body, there is an advantage in that the type of tool may be quickly determined and an operation of the scanner matching the type of tool may be performed without a separate operating process.

Further, when a control unit determines the type of tool to be a calibration tool, the control unit controls to automatically execute the calibration application to be displayed on a display unit and thus a process of requiring the user to execute a separate calibration application and select a calibration start button may be omitted, which provides an advantage of minimizing user inconvenience.

Further, the control unit distinguishes an auto-calibration tool and a manual calibration tool and performs control matching to a type of tool, thus providing an advantage of minimizing user inconvenience.

Further, when the auto-calibration tool is attached to the scanner body, the control unit applies a control signal to the auto-calibration tool so as to control a location of a pattern plate to be an initial location thereof, thus providing an advantage of performing accurate calibration of the scanner body through the auto-calibration tool.

### BRIEF DESCRIPTION OF DRAWINGS

The present disclosure may be easily understood through the following detailed description and the accompanying drawings, in which reference numerals refer to structural elements.

FIG. 1 is a view illustrating at least a portion of configurations of an image processing apparatus according to an embodiment of the present disclosure.

FIG. 2 is an example of a scanner body and a scanning tool detachably coupled to one end of the scanner body.

FIG. 3 is a cross-sectional view of a state in which a scanning tool is coupled to a scanner body.

FIG. 4 is a view illustrating an operation of photographing an object by an image processing apparatus according to the present disclosure.

FIG. 5 is an example of a scanner body and a calibration tool detachably coupled to one end of the scanner body.

FIGS. 6 and 7 are exploded views of a scanner body and a calibration tool detachably coupled to one end of the scanner body.

FIG. 8 is a cross-sectional view of a state in which a calibration tool is coupled to a scanner body.

FIG. 9 is a schematic view of a scanner body corresponding to a configuration of an image processing apparatus according to the present disclosure.

FIG. 10A is an example of image data acquired when a scanning tool is attached to an end of a scanner body and FIG. 10B is an example of image data acquired when a calibration tool is attached to an end of a scanner body.

FIG. 11 is an example of identification targets formed on a pattern plate of a calibration tool and a sign formed by the identification targets.

FIG. 12 is an example illustrating a process of performing calibration of a scanner body by using a calibration tool.

FIG. 13A is an example illustrating an auto-calibration tool, and FIG. 13B is an example illustrating three-dimensional information of identification targets which form a sign of the auto-calibration tool.

FIG. 14A is an example illustrating a manual calibration tool, and FIG. 14B is an example illustrating three-dimensional information of identification targets which form a sign of the manual calibration tool.

FIGS. 15A and 15B are examples illustrating a process of performing calibration of a scanner body by using a manual calibration tool.

FIG. 16 is an exemplary flowchart illustrating an image processing method using an image processing apparatus according to the present disclosure.

FIG. 17 is an example illustrating a tool distinguishing operation of an image processing method using an image processing apparatus according to the present disclosure.

FIG. 18 is another example illustrating a tool distinguishing operation of an image processing method using an image processing apparatus according to the present disclosure.

FIG. 19 is an example illustrating a scanner control operation of an image processing method using an image processing apparatus according to the present disclosure.

FIG. 20 is an example illustrating a calibration performance operation of an image processing method using an image processing apparatus according to the present disclosure.

BRIEF DESCRIPTION OF REFERENCE NUMERALS

| | |
|---|---|
| 1: Image processing apparatus | 100: Tool |
| 101: Scanning tool | 102: Calibration tool |
| 200: Scanner body | 500: External electronic apparatus |
| 510: Display unit | 610: Target |
| 611: Calibration target | 612: Identification target |
| 700: Sign | |
| S110: Tool attachment operation | S120: Tool attachment/detachment |
| | identification operation |
| S130: Tool distinguishing operation | S140: Scanner control operation |

### DETAILED DESCRIPTION

Hereinafter, some embodiments of the present disclosure will be described in detail with reference to exemplary drawings. In allocating reference numerals to components of individual drawings, the same component may have the same reference numeral even though the same component is displayed on different drawings. Furthermore, in describing some embodiments of the present disclosure, a detailed description of known elements or functions will be omitted if it is determined that the detailed description hinders understanding of the embodiments of the present disclosure.

In describing the components of an embodiment, terms such as first, second, A, B, (a), and (b) may be used. These are used solely for the purpose of differentiating one component from another and do not imply or suggest the essence, order or sequence of the components. In addition, unless defined otherwise, all technical and scientific terms used herein have the same meaning as commonly understood by a person of ordinary skill in the art to which the inventive concept belongs. It will be further understood that terms, such as those defined in commonly used dictionaries, should be interpreted as having a meaning that is consistent with their meaning in the context of the relevant art and should not be interpreted in an idealized or overly formal sense unless expressly defined herein.

The term “part (portion)” used in the specification may be implemented in software or hardware, and it is possible that multiple “parts” are implemented in one unit (element) or one “part” includes multiple components depending on the embodiments.

In describing the present disclosure, the expression “configured to” used in an embodiment of the disclosure may be used interchangeably with “suitable for,” “having the capacity to,” “designed to,” “adapted to,” “made to,” or “capable of” depending on a situation. The term “configured to” may not necessarily refer to “specifically designed to” in a hardware manner. Instead, in some situations, the expression “a system configured to” may refer to what the system is “capable of” operating in conjunction with other apparatuses or components. For example, the phrase “a processor configured to perform A, B, and C” may refer to a dedicated processor (e.g., an embedded processor) to perform the corresponding operation” or a generic-purpose processor (e.g., a CPU or application processor) capable of performing the corresponding operation by executing one or more software programs stored in a memory.

In an embodiment disclosed in the present disclosure, the three-dimensional scanner refers to an electronic apparatus configured to acquire an image related to an object. Specifically, the three-dimensional scanner referred to in the present disclosure may refer to a scanner configured to acquire an image related to an oral cavity, which is used for oral treatment. For example, the three-dimensional scanner in an embodiment of the present disclosure may include a hand-held intraoral scanner having a shape which may be inserted into an actual oral cavity of a patient.

Hereinafter, for convenience of explanation, the hand-held intraoral scanner having a shape which may be inserted into an actual oral cavity is referred to as the “three-dimensional scanner.”

In an embodiment disclosed in the present disclosure, an image may refer to an image (e.g., “an intraoral image”) representing an object included in an oral cavity. In this case, the object may include a tooth, a gingiva, and at least a partial portion of the oral cavity, and/or an artificial structure (e.g., an orthodontic apparatus including a bracket and a wire, an implant, a denture, a dental restoration including an inlay and an on-lay, and an orthodontic aid inserted into the oral cavity) insertable into the oral cavity. In addition, the object may also include an artifact related to the oral cavity, such as a plaster model, a crown, or the like. In addition, the orthodontic apparatus may include at least one of a bracket, an attachment, an orthodontic screw, a lingual orthodontic apparatus, and a removable orthodontic retainer.

In an embodiment disclosed in the present disclosure, the image may refer to an image showing the inside of the tool. That is, the image may refer to an image showing the inside of the calibration tool required for performing calibration of the three-dimensional scanner.

Furthermore, the image in an embodiment of the present disclosure may include a two-dimensional image with respect to the object or a three-dimensional model or three-dimensional image representing the object three-dimensionally.

Furthermore, the image in an embodiment of the present disclosure may refer to data required for presenting the object in two or three dimensions, for example, raw data or a raw image acquired from at least one camera. Specifically, the raw image may refer to data acquired to generate an intraoral image required for diagnoses and may include an image (e.g., a two-dimensional frame image) acquired by at least one camera included in the three-dimensional scanner when scanning the patient's oral cavity using the 3D scanner. In addition, the raw image may correspond to an unprocessed image and may refer to an original image acquired from the intraoral scanner.

Hereinafter, embodiments of the present disclosure will be described in detail with reference to the drawings.

FIG. 1 is a view illustrating at least a portion of configurations of an image processing apparatus 1 according to an embodiment of the present disclosure. Referring to FIG. 1, the image processing apparatus according to the present disclosure includes a tool 100 and a scanner body 200. The tool 100 may be detachably coupled to one end of the scanner body 200. The tool 100 may be attached to the scanner body 200 or detached from the scanner body 200 to allow a predetermined operation of the scanner body 200 to be performed.

By coupling a specific type of tool 100 to one end of the scanner body 200, the scanner body 200 may acquire a three-dimensional model by scanning an object (not shown). According to an embodiment disclosed in the present disclosure, the tool 100 may correspond to a scanning tool 101, and when the scanning tool 101 is coupled to one end of the scanner body 200, a three-dimensional model may be acquired by the scanner body 200 scanning the object. Here, the object may refer to an object for which the user desires to acquire a three-dimensional model, and for example, the object may correspond to at least one of the inside of the patient's oral cavity, a negative model modeled after the inside of the patient's oral cavity, and a positive model acquired by pouring plaster on the negative model. The scanner body 200 may correspond to a component of the three-dimensional scanner, which is included in the three-dimensional scanner to scan the object and acquire the three-dimensional model.

The scanner body 200 may include a scanner body case 201. The scanner body case 201 may function to safely protect components built into the scanner body 200 from the external environment. By way of example, the body case 201 may be formed of a shape and material that is easy for the user to grasp by hand, but the shape and material of the body case 201 are not particularly limited.

For example, the body case 201 may include an upper case 201a and a lower case 201b, and a combination structure of the upper case 201a and the lower case 201b may allow the components built into the scanner body 200 to be protected from the external environment. However, the body case 201 may not be formed by the combination structure of the upper case 201a and the lower case 201b and may have a multi-stage combination structure or an integrated structure.

The scanner body 200 and an external electronic apparatus 500 may be connected to each other in communication. More specifically, the three-dimensional scanner included in the scanner body 200 may correspond to a medical apparatus for acquiring an image inside the oral cavity. The three-dimensional scanner insertable into an oral cavity, such as the intraoral scanner shown in FIG. 1, may be referred to as a hand-held intraoral scanner, a portable scanner, or the like.

More specifically, the intraoral scanner of the three-dimensional scanner may correspond to an apparatus which is inserted into the oral cavity and scans teeth in a non-contact manner to generate a three-dimensional model with respect to the oral cavity including at least one tooth. The intraoral scanner corresponding to one type of three-dimensional scanner may have a shape which may be inserted into or drawn out from the oral cavity and may scan the inside of the oral cavity of the patient by using at least one camera (e.g., an optical camera). In order to image a surface of at least one of objects, such as a tooth, a gingiva inside the oral cavity, an artificial structure (e.g., an orthodontic apparatus including a bracket, a wire, and the like, an implant, a denture, and an orthodontic aid inserted into the oral cavity) insertable into the oral cavity, and a plaster model, the three-dimensional scanner may acquire surface information with respect to the object as raw data.

Here, the raw data acquired by the three-dimensional scanner may include at least one image acquired by at least one camera included in the three-dimensional scanner. Specifically, the raw data may include at least one two-dimensional frame image acquired through intraoral scanning by the three-dimensional scanner. In this case, the “image frame” may be referred to as a “frame” or “frame data.”

The raw data acquired by the three-dimensional scanner may be transmitted to the external electronic apparatus 500 connected through a communication network.

The three-dimensional scanner may acquire a three-dimensional model or three-dimensional image generated based on the raw data acquired by the at least one camera. The acquired three-dimensional model or three-dimensional image may be transmitted to the external electronic apparatus 500.

The external electronic apparatus 500 may be connected to the three-dimensional scanner through a communication network and may receive data (e.g., image data) acquired by scanning the object from the three-dimensional scanner. The external electronic apparatus 500 may include any electronic apparatuses which may generate, process, display, and/or transmit an intraoral image based on the data transmitted from the three-dimensional scanner.

More specifically, the external electronic apparatus 500 may generate at least one of information required for oral diagnosis and an image representing the oral cavity based on the data received from the three-dimensional scanner, and display the generated information and image through a display unit 510. The display unit 510 may be a display apparatus visually displaying received data and/or data processed from the received data, and the display unit 510 may correspond to at least one of known display apparatuses (e.g., a monitor, a tablet, a screen, or the like).

For example, the external electronic apparatus 500 may include any electronic apparatuses which may generate, process, display, and/or transmit three-dimensional data or a three-dimensional image based on the image data received from the intraoral scanner.

For example, the external electronic apparatus 500 may include a computing apparatus, such as a smartphone, a laptop computer, a desktop computer, a PDA, a tablet PC, etc., but is not limited to the listed examples.

In addition, the external electronic apparatus 500 may exist in the form of a server (or server apparatus) for processing an image of an object (e.g., an intraoral image).

The external electronic apparatus 500 may store and execute dedicated software linked to the intraoral scanner. Here, the dedicated software may be referred to as a dedicated program or dedicated application. In case that the external electronic apparatus 500 operates in conjunction with the intraoral scanner, the dedicated software stored in the external electronic apparatus 500 may be connected to the intraoral scanner and receive data acquired through object scanning in real time. For example, dedicated software for processing data for each intraoral scanner product may exist. The dedicated software may perform at least one operation for acquiring, processing, storing, and/or transmitting a three-dimensional image of the object.

The three-dimensional scanner may transmit the raw data acquired by scanning the object to the external electronic apparatus 500 as it is. Thereafter, the external electronic apparatus 500 may generate a three-dimensional object image representing the object in three dimensions based on the received raw data. In addition, the external electronic apparatus 500 may generate a “three-dimensional intraoral image” by modeling an internal structure of the oral cavity in three-dimensions based on the received raw data, and therefore, data thus generated may be referred to as the “three-dimensional intraoral model.”

The external electronic apparatus 500 may receive a command through an input unit (not shown) electrically connected thereto. For example, the input unit may include at least one of known input apparatuses such as a keyboard, a mouse, and a scanner. By way of example, when the user inputs a calibration command through the input unit, a processor (not shown) built into the external electronic apparatus 500 may control an operation of the scanner body 200 so that the scanner body 200 performs the calibration process. A detailed description of the calibration process will be provided later.

Meanwhile, the aforementioned “three-dimensional scanner” or “intraoral scanner” includes the scanner body 200 and thus an operation of the “three-dimensional scanner” or “intraoral scanner” may correspond to an operation of the scanner body 200.

In addition, for example, the image processing apparatus according to an embodiment disclosed in the present disclosure may include a user interface, and the user interface may include an input apparatus including keys corresponding to a predetermined operation or command, or the like. For example, the input apparatus included in the user interface may include at least one button, a contact sensor, or the like. Alternatively, the user interface may include a voice recognition sensor and may receive a user's voice and recognize a user input corresponding to a predetermined operation or command based on the received user's voice. As shown in FIG. 2, the user interface included in scanner body 200 of the image processing apparatus may be formed with at least one button 290. In FIG. 2, it is exemplified that the user interface includes two buttons 291 and 292.

As another example, the user interface may be formed as a touch pad. In detail, the user interface may include a touch pad (not shown) to be coupled to a display panel (not shown). Here, a user interface screen may be output on the display panel. When a predetermined command is input through the user interface screen, the touch pad may detect information thereof and transmit the detected information to a scanner-side processor (210 in FIG. 9 described below). Thereafter, the scanner-side processor may interpret the detected information and recognize and execute the predetermined command input by the user.

More specifically, when the user interface includes the touch pad and the user touches a predetermined point of the user interface screen, the user interface may detect a location of the touched point. Thereafter, the detected location information may be transmitted to the scanner-side processor. The scanner-side processor may then recognize a user's request or command corresponding to a menu displayed on the detected location and execute the recognized request or command.

Hereinafter, a case in which the user interface includes multiple buttons 291 and 292 as shown in FIG. 2 to be described below will be provided as an example.

As shown in FIG. 2, the user interface may include a first button 291 among the buttons 290. When the user interface is formed as the first button 291, depending on a type in which the first button 291 is pressed, the user input corresponding to an operation of the first button 291 may correspond to at least one request. For example, a user operation with respect to the first button 291 may be distinguished as a double click, a long click, a single click, or a one-time click for a short period of time, and may be recognized as different requests depending on the distinguished user operation type.

By way of example, when the user briefly presses the first button 291 once, the scanner-side processor may recognize that a user input requesting to start scanning of the object is received. When the user presses the first button 291 once for a long time (or, for a configured time or longer), the scanner-side processor may recognize that a user input requesting termination of scanning the object is received. Alternatively, when the user double-clicks the first button 291, the scanner-side processor may recognize that a user input requesting transmission of image data corresponding to acquired images to the external electronic apparatus 500 is received.

As another example, different requests may be recognized in consideration of an operation state of the scanner body 200 when a user operation is performed on the first button 291. For example, when the first button 291 is briefly pressed once while the scanner body 200 performs scanning, the user input may be recognized as a request corresponding to the termination of the scanning. When the first button 291 is briefly pressed once in a state in which the scanner body 200 stops the scanning, the user input may be recognized as a request corresponding to a restart of the scanning.

Further, the user interface of the scanner body 200 of the image processing apparatus may include multiple buttons 291 and 292 corresponding to multiple requests, respectively. In this case, a corresponding request may be recognized depending on a selected button. For example, a second button 292 formed to be spaced a predetermined distance away from the first button 291 may serve as a cursor on the display unit 510. Accordingly, the user may operate an application (e.g., the calibration application) displayed on the display unit 510 by using the first button 291 and the second button 292 formed on the scanner body 200 without having a separate input apparatus.

Hereinafter, a tool 100 which is a component of the image processing apparatus according to an embodiment disclosed in the present disclosure will be described in detail.

FIG. 2 is an example of a scanner body 200 and a scanning tool 101 detachably coupled to one end of the scanner body 200, and FIG. 3 is a cross-sectional view of a state in which the scanning tool 101 is coupled to the scanner body 200.

Referring to FIGS. 1 to 3, a type of the tool 100 which is a component of the image processing apparatus according to an embodiment disclosed in the present disclosure may vary depending on use. For example, in order to scan the object and acquire a three-dimensional model representing the object, the tool 100 coupled to an end of the scanner body 200 may correspond to the scanning tool 101. The scanning tool 101 may be formed to have a shape for guiding reflection light reflected from a surface and/or the inside of the object to the scanner body 200.

By way of example, the scanning tool 101 may be formed to have a shape including an opening on one side to guide light emitted toward the object from the scanner body 200 or guide reflection light reflected from the surface and/or the inside of the object to the scanner body 200. For example, the scanning tool 101 may include a scanning tool-side coupling part 1011 coupled to an end of the scanner body 200 and an opening part 1012 formed on one side of the other end opposite to the scanning tool-side coupling part 1011.

Meanwhile, the scanning tool 101 may include a light path change member 1013 to guide output light inside thereof toward the opening part 1012 or guide reflection light incident through the opening part 1012 toward the scanner body 200. The light path change member 1013 may reflect and/or refract light and the light path change member 1013 may correspond to an optical component, such as a mirror, a lens, or a prism.

Hereinafter, an operation of photographing the object in the scanner body 200 which corresponds to a component of the image processing apparatus 1 according to an embodiment disclosed in the present disclosure will be described in detail.

FIG. 4 is a view illustrating an operation of photographing an object by the image processing apparatus 1 according to the present disclosure.

Referring to FIG. 4, the scanner body 200 may include at least one camera 232 and the camera 232 may perform photographing with respect to the oral cavity and acquire at least one image. More specifically, the camera 232 may perform photographing with respect to the oval cavity to generate image data to be transmitted to an external electronic apparatus (not shown) (e.g., corresponding to 500 in FIG. 1).

Here, the image data generated in the camera 232 may correspond to at least one image itself, which is acquired by the at least one camera 232.

Alternatively, the camera 232 may generate image data corresponding to the acquired at least one image. Alternatively, the camera 232 may change a shape of the acquired at least one image to generate image data. Alternatively, the image data generated in the camera 232 may correspond to a three-dimensional image or a three-dimensional model representing the object in three dimensions based on multiple images acquired by the at least one camera 232. Hereinafter, for convenience of explanation, the “at least one camera 232” will be referred to as the “camera 232.” That is, the camera 232 may be referred to as one camera or multiple cameras.

The camera 232 may include at least one image sensor (not shown). Specifically, at least one camera included in the camera 232 each may include a lens 2321 and an image sensor (not shown). Here, the image sensor (not shown) may correspond to an apparatus which converts light incident through the lens 2321 into an electric signal to show an image for acquiring the image. For example, the image sensor may correspond to at least one of known image sensors such as a CCD sensor, CMOS sensor, or a color image sensor, but is not necessarily limited to the examples listed. The image sensor may be included in an imaging board 2322 disposed inside the camera, and the imaging board 2322 may be electrically connected to the lens 2321.

The camera 232 may acquire hundreds of images per second depending on a configured frames per second (FPS). Here, the image acquired by the camera 232 may correspond to a two-dimensional frame image. The FPS indicates the number of frame images acquired per second and may be referred to as a “frame rate.”

For example, when an operation frame per second (FPS) of the camera 232 is 100 FPS, the camera 232 may acquire 100 object images per second. For example, in case that the camera 232 of the scanner body 200 includes two cameras, an L camera (left camera) 232a and an R camera (right camera) 232b, 100 images may be acquired per second by the L camera 232a and 100 images may be acquired per second by the R camera 232b. Further, since the L camera 232a and the R camera 232b operate synchronized, the L camera 232a and the R camera 232b may concurrently acquire an L image and an R image, respectively.

As another example, in case that the camera 232 of the scanner body 200 includes one camera, 100 images may be acquired per second.

As yet another example, in case that the scanner body 200 performs image scanning in a confocal manner, each of the at least one camera included in the camera 232 may include a lens (not shown) configured to be movable to adjust a location of a focus and an image sensor (not shown) for acquiring an image based on light passing through the lens (not shown).

As still another example, in case that the scanner body 200 performs scanning in an optical triangle manner, each of the at least one camera included in the camera 232 may perform image scanning with respect to the object O to which the pattern is emitted.

Meanwhile, the camera 232 of the scanner body 200 may include, in addition to the at least one camera 232 for acquiring at least one image, an imaging board 2322 for acquiring image data corresponding to the at least one image.

Further, the imaging board 2322 may control the camera 232 for image scan. By way of example, the imaging board 2322 may configure a region of interest (ROI), an exposure time, a frame rate and/or the like of the camera 232.

Alternatively, the imaging board 2322 may generate image data corresponding to the at least one image acquired by the lens 2321. For example, the imaging board may convert a format of the at least one image acquired through the lens 2321 to generate image data corresponding to the at least one image.

Alternatively, the imaging board 2322 may generate image data corresponding to multiple images by encoding the at least one image acquired by the lens 2321.

In addition, in case that the camera 232 does not include the imaging board 2322, at least one of the aforementioned operations performed by the imaging board 2322 may be performed by a scanner-side processor 110 described below.

In an example shown in FIG. 4, a case in which the camera 232 included in the three-dimensional scanner 100 includes two cameras 232a and 232b and performs image scanning in an optical triangle manner is shown and described as an example.

Referring to FIG. 4, the three-dimensional scanner 100 may perform intraoral scanning using an optical triangle method. The optical triangle method may allow capturing an image by emitting light to the object, and acquiring three-dimensional data using the image of the object imaged with light. Specifically, by the optical triangle method, light is emitted to the object O and the light reflected by the object O is received so as to acquire an image with respect to the object O. Here, the three-dimensional data may include three-dimensional depth information with respect to the object O.

More specifically, in order to acquire the three-dimensional data with respect to a surface of the object O in an embodiment disclosed in the present disclosure, a structured light with stereo vision method using two cameras and a projector 231 outputting light may be used.

For example, in order to perform scanning by using the structured light with stereo vision method, the scanner body 200 may include an optical part 230, and the optical part 230 may include a projector 231 in addition to the camera 232 described above. Here, the projector 231 may emit, to the tool 100 side, output light having a pattern formed by at least one of a one-dimensional point and a two-dimensional line. Specifically, in order to scan an oral cavity which is one type of the object O, the projector 231 may emit output light into the oral cavity which is the object O by controlling of the scanner-side processor 210 described below. When the output light is emitted from the projector 231, two or more cameras 232a and 232b may receive reflection light reflected from the surface of the object O to which the output light has been emitted and acquire an image corresponding to the object O. Furthermore, a shape (or pattern) of the light output from the projector 231 may be changed and the light may have various shapes. In FIG. 4, a case in which the projector 231 outputs structured light p in a form of multiple lines is exemplified.

The scanner body 200 which is a component of the image processing apparatus 1 according to an embodiment disclosed in the present disclosure may emit the structured light p to the object O and the first camera 232a corresponding to a left field of view and the second camera 232b corresponding to a right field of view may acquire a first image 400 or 401 corresponding to the left field of view and a second image 400 or 402 corresponding to the right field of view, respectively. The scanner body 200 may continuously acquire two-dimensional frame images including the first image 400 or 401 and the second image 400 or 402 with respect to the object O. For example, in case that the camera 232 operates in 100 frames per second (FPS), the first camera 232a and the second camera 232b each may continuously capture 100 frame images per second. Here, the frame images acquired by the camera 232 may correspond to two-dimensional images corresponding to a resolution of the camera 232.

Further, the multiple frame images acquired by the two or more cameras 232a and 232b may be formatted in an imaging board (e.g., 2322 in FIG. 9 described below) into a different image format, for example, an HDMI format. Accordingly, an external electronic apparatus (e.g., 500 in FIG. 1) may receive HDMI data acquired by formatting the images acquired by the first camera 232a and the second camera 232b. In addition, the external electronic apparatus (e.g., 500 in FIG. 1) may reconstruct a three-dimensional frame representing a surface shape of the object O from the two-dimensional frame including the first image 400 or 401 and the second image 400 or 402. Specifically, depth information with respect to the object may be acquired based on the first image 400 or 401 and the second image 400 or 402 in which the structured light p is imaged together. Furthermore, a three-dimensional image or a three-dimensional model may be reconstructed based on the depth information.

The imaging board or the scanner-side processor may generate a three-dimensional image or a three-dimensional model with respect to the object based on the multiple frame images acquired by the two or more cameras 232a and 232b. Here, the scanner body 200 may transmit the generated three-dimensional image or three-dimensional model to the external electronic apparatus (e.g., 500 in FIG. 1) through a second communication network.

In FIG. 4, a case in which the scanner body 200 includes two cameras 232a and 232b and one projector 231 is exemplified. However, embodiments are not limited to the example shown in FIG. 4, and the scanner body 200 may include one camera and one projector. In case that the scanner body 200 includes one camera and one projector, the projector may concurrently function as a camera acquiring an image and a projector emitting structured light. Depending on various implementation methods of the present disclosure, the scanner body 200 may include multiple cameras and multiple projectors.

The scanner body 200 may move around the object O and scan the object O at predetermined time intervals (e.g., several ms to tens of ms) to acquire at least one image (e.g., multiple two-dimensional frames). The scanner body 200 or the external electronic apparatus (not shown) (e.g., 500 in FIG. 1) may acquire multiple three-dimensional frames from the multiple two-dimensional frames. For example, at least one image sensor which includes the camera 232 included in the scanner body 200 may each acquire tens to hundreds of two-dimensional frames per second. Specifically, the image sensor may acquire tens to hundreds of three-dimensional frames per second by using the multiple two-dimensional frames acquired from the scanner body 200. Here, each three-dimensional frame may be generated based on the multiple two-dimensional frames. For example, one three-dimensional frame may be generated based on dozens of two-dimensional frames.

In case that the external electronic apparatus (not shown) (e.g., 500 in FIG. 1) receives image data corresponding to the two-dimensional frames acquired from the scanner body 200, the external electronic apparatus may reconstruct a three-dimensional image or three-dimensional model with respect to the entirety of the object by combining or arranging the locations of multiple three-dimensional frames based on the received image data.

For example, each of the first camera 232a and the second camera 232b may acquire 100 or more two-dimensional frames per second. The first camera 232a and the second camera 232b may capture an image having a resolution of M*N. Here, M and N may have natural number values, M may represent the number of horizontal pixels of the acquired image, and N may represent the number of vertical pixels of the acquired image.

Hereinafter, for convenience of explanation, a case in which each of at least one image acquired by each of at least one image sensor (not shown) included in the camera 232 (e.g., the first camera 232a and the second camera 232b) corresponds to a two-dimensional frame formed by pixel values of 200 pixels in width and 200 pixels in height (i.e., M=200, and N=200) will be described and explained. In the example described above, although a case in which M and N have the same value has been exemplified, M and N may have different natural number values.

Further, one pixel value may be expressed as 8 bits. In this case, each of the frame images acquired by each of the first camera 232a and the second camera 232b may correspond to image data having a size or resolution of 200×200×8 bit=4,000 bytes.

A camera board may format a format of the multiple images acquired by the first camera 232a and the second camera 232b in a high-definition multimedia interface (HDMI) to generate image data (specifically, HDMI data). Here, the HDMI data may include 2K data, 4K data, or 8K data having the HDMI format. Here, the HDMI format may have an image frame form having a resolution defined in the HDMI standard and may have formats such as 1,920×1,080=2K resolution, 4,096×2,160=4K resolution, and 7,680×4,320=8K resolution.

Hereinafter, for convenience of explanation, a resolution of an image acquired from a camera (e.g., the first camera 232a and the second camera 232b) included in the scanner body 200 is referred to as a “first resolution” and a resolution of image data having the HDMI format is referred to as “second resolution.”

The first resolution may refer to a total resolution of at least one image acquired at the same time point by the camera 232 included in the scanner body 200. For example, if the intraoral scanner (e.g., 200 or 201) includes two cameras, the first camera 232a and the second camera 232b, the first image 400 or 401 and the second images 400 or 402 may be acquired at the same time point. In addition, in case that each of the two cameras 232a and 232b has a resolution of 200 pixels in width and 200 pixels in height, when two images acquired at once from the same time point by the two cameras 232a and 232b are combined in the horizontal direction, the image acquired by combining the two images may be expressed as having a resolution of 400 pixels in width and 200 pixels in height. That is, when two images acquired from the two cameras 232a and 232b included in the scanner body 200 are expressed as one image, the image having a resolution of 400 pixels in width and 200 pixels in height may be acquired. Hereinafter, for convenience of explanation, one image acquired by combining two images acquired from the two cameras 232a and 232b at the same time point will be referred to as a “raw image.”

In the example described above, the first resolution may correspond to a value acquired by multiplying 200 pixels in width by 200 pixels in height or a value acquired by multiplying 400 pixels in width by 200 pixels in height, and the second resolution may correspond to 2K, 4K, 8K, and the like.

As shown in the example described above, the scanner body 200, which is a component of the image processing apparatus 1 according to an embodiment disclosed in the present disclosure, may generate HDMI data including the pixel values of the frame images acquired from each of the first camera 232a and the second camera 232b as they are and transmit the HDMI data to an external electronic apparatus (not shown) (e.g., 500 in FIG. 1). Specifically, the scanner body 200 may format the pixel values of the frame images acquired from each of the first camera 232a and the second camera 232b in the HDMI format so that pixel values of frame images acquired from each of the first camera 232a and the second camera 232b are included as they are.

Hereinafter, a calibration tool 102, which is another type of the tool 100 of the image processing apparatus 1 according to an embodiment disclosed in the present disclosure will be described.

FIG. 5 is an example of a scanner body 200 and a calibration tool 102 detachably coupled to one end of the scanner body 200, FIGS. 6 and 7 are exploded views of the scanner body 200 and the calibration tool 102 detachably coupled to one end of the scanner body 200, and FIG. 8 is a cross-sectional view of a state in which the calibration tool 102 is coupled to the scanner body 200.

Referring to all of FIGS. 5 to 8, the tool 100, which is a component of the image processing apparatus 1 according to an embodiment disclosed in the present disclosure, may include the calibration tool 102. The calibration tool 102 may correspond to a component enabling calibration of at least some (particularly, the optical part) of components built in the scanner body 102, and may be detachably coupled to one end of the scanner body 200.

By way of example, the user may detach the scanning tool 101 which has been coupled to the scanner body 200 and attach the calibration tool 102, which is provided separately, thereto so as to perform calibration of the scanner body 200. When the user attaches the calibration tool 102 to the scanner body 200, an attachment/detachment surface 202, which is formed on at least a portion of the scanner body 200 and configured to couple the tool 100 to the scanner body 200, and a connection block 203 protruding from the attachment/detachment surface 202 by a predetermined length may be inserted into the calibration tool 102 and placed thereon. Since the attachment/detachment surface 202 and the connection block 203 are inserted into the calibration tool 102 and placed thereon, it is possible to stably calibrate the scanner body 200.

Hereinafter, a detailed configuration of the calibration tool 102 will be described more specifically. However, the detailed configuration of the calibration tool 102 may be changed, added, or removed as needed, and is not limited by the detailed description of the present disclosure described as an example.

In the image processing apparatus 1 according to an embodiment disclosed in the present disclosure, the calibration tool 102, which is one type of the tool 100, may include a calibration tool body 110 which is inserted into and seated on one end of the scanner body 200 in a state in which the scanning tool 101 is removed and includes a body insertion hole 111 formed thereon, a calibration pattern plate 1955 (hereinafter, referred to as a “pattern plate”) disposed inside the calibration body 110 to scan and correct the scanner body 200, and a pattern moving part 190 which automatically performs at least one of axis rotation movement and axis direction movement of the pattern plate 1955 inside the calibration tool body 110 when the scanner body 200 is coupled to the calibration tool body 110. Here, when one end of the scanner body 200 is inserted into the calibration tool body 110, the pattern plate 1955 may be disposed to face the camera provided inside the scanner body 200. In addition, the axis direction in which the pattern plate 1955 moves may correspond to a longitudinal direction of the scanner body 200. The calibration tool 102 may include a tool which may automatically perform the axis rotation movement and the axis direction movement of the pattern plate 1955.

For example, the calibration tool body 110 may have a flat lower surface so as to be more stably supported and seated on a desk or table where calibration is performed. In addition, the calibration tool body 110 may have a lower surface having an area relatively smaller than that of an upper surface, but is not limited thereto. The upper and lower surfaces of the calibration tool body 110 may be formed to have rounded edges as a whole. As another example, the calibration tool body 110 may have a cylindrical shape as a whole.

An emission path of the output light emitted from the projector (e.g., 231 in FIG. 4 described above and FIG. 9 described below) of the scanner body 200 may be provided inside the calibration tool body 110 and in particular, the emission path of the output light and the reflection light may be provided in a dark room form so as not to be affected by external light. That is, the calibration tool 102 is formed into a shape having one side closed, and when one end of the scanner body 200 is inserted into and seated on the scanner body insertion hole 111, the inside of the calibration tool 102 forms a dark room, and the output light emitted from the projector 231 of the scanner body 200 and the reflection light reflected from the pattern plate 1955 exist inside the calibration tool 102.

The calibration tool body 110 may include a main printed circuit board 150 which configures the lower surface of the calibration tool body 110 and shields the inside thereof in a dark room form.

On the main printed circuit board 150, a scanning location detection part to be described below may be mounted and disposed and a power supply line (not shown) for supplying external power may be printed. However, the lower surface of the calibration tool body 110 does not necessarily need to be provided in the form of a PCB, such as the above-described main printed circuit board 150, and may be provided in various other forms as long as long as it is possible to supply power to a specific component (e.g., scanning location detection part).

Further, the main printed circuit board 150 may include a motor control part (not shown) mounted and disposed in a MICOM form for controlling an operation of a driving motor 191 among configurations of the pattern moving part 190 to be described below.

A motor PCB 180 for controlling operation and supplying power of the driving motor 191 among configurations of the pattern moving part 190 may be provided in a sub-PCB form inside the calibration tool body 110. The motor PCB 180 may include an external power connector 185 capable of supplying external power through a wired connection and an internal power connector 181 mounted and disposed thereon for power connection to a display PCB 160 described below.

In addition, the display PCB 160 for displaying an operating state of the calibration tool 102 may be further provided inside the calibration tool body 110.

The display PCB 160 may be disposed to be in close contact with an inner upper surface of the calibration tool body 110, and a power on/off indicator lighting 163a and an operating state indicator lighting 163b may be provided in an LED form on an upper surface of the display PCB 160. In addition, a power supply connector 161 for wired connection with the internal power connector 181 provided on one side of the motor PCB 180 may be provided on one side of an edge of the display PCB 160 as described above.

A power on/off indicator hole 113a and an operation status indicator hole 113b, which allow the light emitted from the power on/off indicator lighting 163a and the operation status indicator lighting 163b to be transmitted to the outside, may be formed in the upper surface of the calibration tool body 110.

Here, light of the operating state indicator lighting 163b mounted and disposed on the display PCB 160 may be emitted to the outside through the operating state indicator hole 113b via a light guide 165 made of a transparent material.

The scanner body insertion hole 111 formed on one side of the calibration tool body 110 in the longitudinal direction may have a form to which the connection block 203 provided at one end (e.g., a front end) of the scanner body 200 in a state in which the scanning tool 101 is removed may be inserted and seated. When the scanning tool 101 is detached from the scanner body 200, the connection block 203 may be exposed to protrude from one end of the scanner body case 201 by a predetermined length.

The calibration tool body 110 may further include an illuminance sensor 169 inside thereof to detect predetermined light. The illuminance sensor 169 may detect that predetermined output light is emitted from the projector 231 into an inner space of the calibration tool body 110 when the scanner body 200 operates. The illuminance sensor 169 may inform the control unit of a time point when the driving motor 191 among configurations of the pattern moving part 190 to be described below may be operated by sensing the output light.

In this case, the illuminance sensor 169 may be mounted and disposed on a lower surface of the display PCB 160 to more accurately measure the output light of the inner space of the calibration tool body 110, but is not limited thereto.

The calibration tool body 110 may further include the scanning location detection part for detecting a location of the pattern plate 1955 inside thereof. The scanning location detection part may detect a location of a mounting block 195 to which the pattern plate 1955 is coupled, thereby providing information so that the control unit may calculate a required distance value and rotation angle value when performing calibration. The scanning location detection part may identify whether the pattern moving part 190 is restored to an initial location at which calibration is performed.

The scanning location detection part performing the function may include, for example, a photo sensor part and a Hall sensor part, but is not limited thereto.

For example, when the scanning location detection part corresponds to a photo sensor part, the photo sensor part may include a photo sensor 170 fixed to the bottom surface of the calibration tool body 110 and a detection lead 175 which is coupled to a moving block 196 or the mounting block 195 to which the pattern plate 1955 is coupled, and rotated and rectilinearly moved.

The detection lead 175 may be coupled to a front edge portion of the moving block 196 or the mounting block 195 among configurations of the pattern moving part 190 and linked and moved together when the moving block 196 or the mounting block 195 performs an axis rotation and/or an axis direction rectilinear movement. In case that the detection lead 175 moves and is inserted into the photo sensor 170, the photo sensor 170 may detect the detection lead 175, detect locations of the moving block 196 and the mounting block 195 to which the detection lead 175 is coupled, and detect a location of the pattern plate 1955 coupled to the mounting block 195. The control unit may calculate a separation distance between the pattern moving part 190 or the pattern plate 1955 and the photo sensor 170 and a rotation angle value through information acquired from the photo sensor part.

As another example, when the scanning location detection part corresponds to a Hall sensor part, the Hall sensor part may include a Hall sensor (not shown) fixed to the calibration tool body 110 and a detection magnet (not shown) which rotates and moves rectilinearly linked to the pattern plate 1955.

The detection magnet may be configured to interact with the Hall sensor through magnetism. The detection magnet may be provided at a front edge portion of the moving block 196 or the mounting block 195 among configurations of the pattern moving part 190 and interlocked and moved together when the moving block 196 or the mounting block 195 performs an axis rotation and/or an axis direction rectilinear movement. In case that the detection magnet moves and is detected by the Hall sensor, the Hall sensor may detect a location of the detection magnet and detect a location of the pattern plate 1955 coupled to the mounting block 195. The control unit may be configured to relatively measure a separation distance between the pattern moving part 190 or the pattern plate 1955 and the Hall sensor and a rotation angle value through information acquired from the Hall sensor part.

When an operation of the scanner body 200 is detected after the scanner body 200 is inserted and seated on the calibration tool body 110 by the illuminance sensor 169, the scanning location detection part may identify the detected location (more specifically, a rotation angle state and a current location of the pattern plate 1955) of the pattern moving part 190 and suggest a reference value for restoring to the initial location if not in the initial calibration location.

The calibration tool main body 110 configured as described above may include a pattern moving part 190 provided inside thereof to move in a horizontal direction. Hereinafter, the “horizontal direction” is defined as indicating a direction parallel to an upper surface of a table on which the calibration tool body 110 is mounted and may be understood as including the longitudinal direction of the calibration tool body 110 and the longitudinal direction of the scanner body 200.

The pattern moving part 190 may be provided to allow the pattern plate 1955 to rotate in the horizontal direction (i.e., the longitudinal direction of the calibration tool body 110) in which one end of the scanner body 200 is inserted and seated as an axis and to reciprocate in the axis direction and may function to automatically move the pattern plate 1955 to enable calibration. Here, the pattern moving part 190 may cause the pattern plate 1955 to simultaneously perform an axis rotation and movement in the axis direction, and while the pattern plate 1955 moves, an angle between optical axes of the output light emitted from the pattern plate 1955 and the scanner body 200 to the pattern plate 1955 may be maintained.

The pattern moving part 190 may be electrically operated by external power supplied through the above-described main printed circuit board 150 or internal power provided in a form like a rechargeable battery inside the calibration tool body 110. In case that the internal power is provided as a rechargeable battery inside the calibration tool body 110, the rechargeable battery may be charged with power in a wired or wireless manner.

The pattern plate 1955 has a predetermined pattern printed or provided for calibration and is provided to perform calibration while being moved linked with the rotation or rectilinear movement of the pattern moving part 190. The pattern plate 1955 may be formed to be tiltable within a predetermined angle range based on the longitudinal direction of the scanner body 200.

The pattern plate 1955 may be disposed to be inclined on a front surface of the mounting block 195 to be described below. To this end, the front surface of the mounting block 195 may be inclined to have a predetermined inclination angle.

Here, the inclination angle of the pattern plate 1955 may be configured to be 40 degrees or more and less than 50 degrees based on the horizontal direction (the longitudinal direction of the scanner body 200). In this case, there is a disadvantage in that, when the pattern plate 1955 is provided to be orthogonal (i.e., 90 degrees) with respect to the horizontal direction, each pattern 255 formed on the pattern plate 1955 has the same depth information (or height information) on the same surface, and thus, in an embodiment of the present disclosure, the pattern plate 1955 is designed to increase the calibration effect by inclining the pattern plate 1955 at a predetermined angle with respect to the horizontal direction.

The pattern moving part 190 may include the driving motor 191 that may be electrically operated and has a rotation shaft, a fixed block 193 fixed to the inside of the calibration tool body 110 and having moving guide holes opened to one side and the other side thereof, and the mounting block 195 to which the pattern plate 1955 is coupled and which is disposed in a moving guide hole in the fixed block 193.

The driving motor 191 may be provided to be fixed adjacent to one end side of the inner space of the calibration tool body 110 where the scanner insertion hole 111 is formed and the other end side opposite to the one end, and to be parallel or coincident with the optical axis. Therefore, it is natural that the axis direction of the pattern plate 1955 moved by the driving of the driving motor 191 may be interpreted as coincident with or parallel to the optical axis of light emitted from the scanner body 200 to the pattern plate 1955. The driving motor 191 may be electrically driven using external power or internal power.

In addition, the pattern moving part 190 may include a transfer block 194 disposed between the driving motor 191 and the mounting block 195 to transfer a rotational force of the driving motor 191 to the mounting block 195.

Here, the transfer block 194 may transfer the rotational force transferred from a rotation shaft 1911 of the driving motor 191 to the mounting block 195, so that the mounting block 195 is configured to rotate within a moving guide hole of the fixed block 193 so as to rotate the pattern plate 1955 disposed to be inclined on an inclined surface of a reception terminal of the mounting block 195 formed in an inclined manner.

The transfer block 194 may include a coupling terminal axially coupled to the rotating shaft of the driving motor 191 and a rotation wing terminal 1945 which is provided at a front end of the coupling terminal and is inserted into a rotation interference hole 1951 formed in the mounting block 195 to cause rotation interference.

Here, when it is assumed that the coupling terminal is formed to have a circular vertical cross section, the rotation wing terminal 1945 may be formed in a wing shape extending to one side and/or the other side farther than an outer circumferential surface of the coupling terminal. When the transfer block 194 is rotated by the rotation wing terminal 1945, it interferes with the mounting block 195, so that the mounting block 195 may be rotated in linkage with the rotation shaft of the driving motor 191.

The rotation interference hole 1951 formed in the mounting block 195 and the rotation wing terminal 1945 of the transfer block 194 may be configured to have vertical cross sections corresponding to each other. In addition, the rotation interference hole 1951 formed in the mounting block 195 may be formed in a shape that interferes with the rotation direction of the rotation wing terminal 1945 and does not interfere with the horizontal direction of the rotation wing terminal 1945.

As such, the rotation interference hole 1951 and the rotation wing terminal 1945 are formed to have vertical cross sections corresponding to each other and have structures that do not interfere with each other in the horizontal direction (i.e., the axis direction), so that the mounting block 195 may be reciprocally moved in the axis direction by the moving block 196 to be described below.

In addition, it is preferable that the rotation interference hole 1951 is formed so that the depth thereof formed in the horizontal direction from the front end of the rotation wing terminal 1945 is greater than or equal to at least a horizontally movable distance of the mounting block 195. This is because the depth of the rotation interference hole 1951 in the horizontal direction is larger than at least the movable distance of the pattern plate 1955, so as to prevent the movement distance from being limited by interference between the mounting block 195 and the transfer block 194.

Here, the rotation shaft 1911 of the driving motor 191 may be stably supported via a center block 192 provided to be supported on the inner surface of the calibration tool body 110, and the transfer block 194 may be axially fixed to the front end of the rotation shaft 1911 of the driving motor 191. The rotation shaft 1911 of the driving motor 191 may be supported for axis rotation by a rotation bearing 1925 interposed in a through-hole 1921 of the center block 192.

Meanwhile, the pattern moving part 190 may further include the moving block 196 which is provided to be linked with the mounting block 195, and rectilinearly moves the mounting block 195 in the horizontal direction by interference with the fixed block 193.

The moving block 196 serves to reciprocate the pattern plate 1955 in the horizontal direction (axis direction) while being rotated in linkage with the mounting block 1950 to which the pattern plate 1955 is coupled.

To this end, the moving block 196 may rotate and rectilinearly move inside the moving guide hole, and the outer circumferential surface of the moving block 196 may include a rotation guide groove formed thereon, in which a front end of a guide member protruding into the moving guide hole of the fixed block 193 is engaged.

The rotation guide groove may be formed on the outer circumferential surface of the moving block 196 and may be machined to be grooved in a spiral shape with a predetermined pitch interval so that the moving block 196 makes at least three rotations.

A pair of guide members may be provided to be spaced apart at intervals of 180 degrees with respect to the center of the moving guide hole of the fixed block 193, but the number and spacing are not limited thereto. One end of the guide member may be inserted into the rotation guide groove provided in a spiral shape.

The mounting block 195 is provided to perform linked rotation with the moving block 196 and reciprocating rectilinear movement in the horizontal direction, and the guide member fixed to the fixed block 193 is provided to be engaged into the rotation guide groove formed on the outer circumferential surface of the moving block 196 which is rotating, so that the rectilinear movement distance of the pattern plate 1955 may be determined according to an amount of rotation of the moving block 196.

However, it is not necessary that the guide member protrudes into the fixed block 193 and the rotation guide groove is provided on the outer circumferential surface of the moving block 196. That is, as described above, if the pattern plate 1955 has a structure capable of rectilinearly moving in the horizontal direction according to the amount of rotation of the moving block 196, the opposite structure may be adopted as well.

More specifically, although not shown in the drawings, the rotation guide groove which allows the guide member to be engaged on an inner circumferential surface of the moving guide hole may be formed, and the guide member may be provided on the outer circumferential surface of the moving block 196.

Meanwhile, the guide member may include a guide bolt, a portion of which is inserted into the rotation guide groove. However, the guide member does not necessarily have to be provided as a guide bolt, and the guide member may correspond to a ball plunger in which a bearing ball is installed. The case in which the guide member is provided as a ball plunger has an advantage of minimizing frictional force due to the rotation of the moving block 196.

On the other hand, contrary to the above description, the calibration tool 102 according to another embodiment of the present disclosure may not include the driving motor, and may include a fixed case for connecting and fixing the scanner body 200, a rotation case rotatably provided with respect to the fixed case, and a moving means provided inside the rotation case and transferring rotation force of the rotation case to rotate and/or rectilinearly reciprocate the pattern plate 1955. That is, the calibration tool 102 may include a manual calibration tool. By way of example, the user may rotate and/or rectilinearly reciprocate the pattern plate 1955 by manipulating the rotation case of the manual calibration tool. As another example, the moving means for rotating and/or rectilinearly reciprocating the pattern plate 1955 may be provided in the form of a dial lever, and the user rotates the dial lever in one direction to rotate and/or rectilinearly reciprocate the pattern plate 1955.

Hereinafter, a detailed configuration of the scanner body 200 which corresponds to a component of the image processing apparatus 1 according to an embodiment disclosed in the present disclosure will be described in detail.

FIG. 9 is a schematic view of a scanner body 200 corresponding to a configuration of the image processing apparatus 1 according to the present disclosure.

Referring to FIGS. 2, 3, 8, and 9, the scanner body 200, which is one component of the image processing apparatus 1 according to an embodiment disclosed in the present disclosure, may include a scanner-side processor 210.

The scanner-side processor 210 may perform at least one control so that the scanner-side processor 210 may perform a desired operation. Specifically, the scanner-side processor 210 may control an operation of photographing (or scanning) with respect to the object, an operation of acquiring an image with respect to the object, and/or an operation of transmitting data (e.g., image data) corresponding to the acquired image. When it is described that the scanner-side processor 210 performs a predetermined operation, the description may indicate not only the case of directly performing the above-described operation by executing at least one control in the scanner-side processor 210, but also the case of controlling other components to perform the above-described operation.

More specifically, the scanner-side processor 210 may include a RAM (not shown) for storing a control signal or data input from the outside of the scanner body 200 or used as a storage area corresponding to various operations performed in the scanner body 200, a ROM (not shown) in which a control program for controlling the scanner body 200 and/or multiple pieces of control-related information are stored, and at least one processor (not shown) (hereinafter, referred to as an “internal processor”) for executing at least one control. The scanner-side processor 210 may be implemented to have a form including at least one internal processor internally and a memory element (e.g., a RAM, a ROM, and the like) for storing at least one of a program, an instruction, a signal, and data to be processed or used by the internal processor.

Further, the scanner-side processor 210 may include a graphic processing unit (not shown) for graphic processing corresponding to a video. The processor 210 may be implemented as a system on chip (SoC) including a core (not shown) and a GPU (not shown) integrated therein. The scanner-side processor 210 may include multiple cores of more than a single core. For example, the scanner-side processor 210 may include a dual-core, a triple-core, a quad-core, a hexa-core, an octa-core, a deca-core, a dodeca-core, a hexadecimal-core, and the like.

In addition, the scanner-side processor 210 may include a designable logic device and a field-programmable gate array (FPGA) which is a semiconductor device including a programmable internal circuit and may perform high-speed image processing using the FPGA.

In detail, the scanner-side processor 210 may acquire image data corresponding to at least one image acquired by the at least one camera 232, control a control signal, which is related to at least one of the operation of acquiring the at least one image and a transmission operation, to be transmitted or received to or from an external electronic apparatus through a scanner-side communication part 240 performing wired or wireless communication, and control the image data to be transmitted to an external electronic apparatus.

The image processing device 1 according to an embodiment disclosed in the present disclosure may include a control unit. The control unit may control an operation of the scanner body 200 in response to the tool 100 to be coupled to the scanner body 200. The control unit may correspond to the scanner-side processor 210 built into the scanner body 200 or a processor of the aforementioned external electronic apparatus (e.g., 500 in FIG. 1).

The scanner body 200 of the image processing apparatus 1 according to an embodiment disclosed in the present disclosure may include an attachment/detachment detection sensor 220. The attachment/detachment detection sensor 220 may be formed on at least a portion of the scanner body 200 to identify attachment/detachment of the tool 100. For example, the attachment/detachment detection sensor 220 may be formed on at least a portion of an attachment/detachment surface 202 formed at one end of the scanner body 200 in order to identify whether the tool is attached or detached. The attachment/detachment detection sensor 220 may correspond to a proximity sensor for identifying that the tool 100 is attached or detached (mounted or removed) to or from the scanner body 200, but is not necessarily limited to the specified example. A process of identifying the type of the tool 100 by operating the optical unit 230 to be described below by the attachment/detachment detection sensor 220, and a process of identifying the type of the tool 100 based on information acquired by the attachment/detachment detection sensor 220 will be described below.

The scanner body 200 of the image processing apparatus 1 according to an embodiment disclosed in the present disclosure may include an optical part 230, and the optical part 230 may include a projector 231 for emitting predetermined light toward an object and a camera 232 for acquiring image data.

The projector 231 may emit predetermined output light and/or a pattern to the object so that the camera 232 may acquire a color and a shape of image data representing the object. The projector 231 may include a light source 2311, and the light source 2311 may operate to acquire a color and a shape of image data. For example, the light source 2311 may include a red light source, a green light source, and a blue light source, and a first color image, a second color image, and a third color image may be acquired by simultaneously or sequentially operating the light sources. The color of the image data may be acquired by combining the color images. However, the light source 2311 is not limited to the listed examples, and may include a light source conforming to various known color acquisition methods.

The light may be emitted to the object with a specific pattern, and the light emitted to the object with the specific pattern may correspond to structured light. A camera module 132 may acquire the shape (i.e., a three-dimensional shape of the object) of image data by acquiring an image of the object to which the structured light is emitted.

The pattern may be generated by a pattern generation part 2312 of the projector 231. Light generated by the light source 2311 may pass through the pattern generation part 2312, and the light passing through the pattern generation part 2312 may be converted into output light corresponding to a shape of the pattern generated by the pattern generation part 2312. For example, the pattern generation part 2312 may correspond to at least one of a pattern mask and a digital micromirror device (DMD), but is not necessarily limited to the listed examples.

The scanner body 200 may include a scanner-side communication part 240 for transmitting and receiving image data and/or a control signal. The scanner-side communication part 240 may perform wireless communication with an external electronic apparatus (e.g., 500 in FIG. 1) through multiple communication channels. Here, the communication channels may refer to a communication network for transmitting and receiving a radio signal through a predetermined frequency band. Specifically, the communication channels may correspond to a communication network through which a radio signal of a frequency band defined according to a predetermined radio communication standard is transmitted and received. Here, the radio communication standard may include a communication standard such as WiGig (Wireless Gigabit), Bluetooth, Wi-Fi, Bluetooth Low Energy (BLE), NFC/RFID, Wi-Fi Direct, UWB, ZIGBEE, or the like. Furthermore, the communication network conforming to a predetermined frequency band, or a predetermined communication standard may be referred to as a communication channel.

The scanner body 200 may further include a scanner-side storage part 250. The scanner-side storage part 250 may store information of the scanner body 200 (a scanner serial number of the scanner or the like) and information related to scanner control. Depending on cases, the scanner-side storage part 250 may store image data generated by an operation of the camera 232. At least one of known recording apparatuses such as a ROM, a RAM, an SSD, an HDD, or a flash memory may be used as the scanner-side storage part 250, and thus detailed descriptions thereof are omitted.

Hereinafter, a process of performing calibration of the scanner body 200 according to detection of attachment and detachment of the tool 100 will be described.

FIG. 10A is an example of image data acquired when a scanning tool 101 is attached to an end of a scanner body 200 and FIG. 10B is an example of image data acquired when a calibration tool 102 is attached to an end of the scanner body 200.

Referring to FIGS. 3, 8, 9, 10A, and 10B, when the tool 100 is attached to or removed (detached) from one end of the scanner body 200, the attachment/detachment detection sensor 220 may detect attachment or detachment of the tool 100. In this case, when the tool 100 is attached to one end of the scanner body 200, the attachment/detachment detection sensor 220 may measure and acquire a distance d between the inner surface of the tool 100 and the attachment/detachment detection sensor 220. In case that the distance d is less than or equal to a predetermined attachment/detachment threshold, it may be determined that the tool 100 is coupled to one end of the scanner body 200. For example, the attachment/detachment threshold may be about 5 mm.

When attachment/detachment of the tool 100 is detected by the attachment/detachment detection sensor 220, the control unit may control the projector 231, and the projector 231 may emit output light toward the tool 100. In this case, the output light may correspond to light in a state in which the above-described pattern generation part 2312 is not applied. Depending on a type of the tool 100 coupled to the scanner body 200, output light emitted from the projector 231 may reach a surface of the object O and/or a surface of the pattern plate 1955. In case that the tool 100 coupled to the scanner body 200 corresponds to the scanning tool 101, the output light emitted from the projector 231 may reach the surface of the object O and reflection light reflected from the surface of the object O may be received in a camera 2321 by an optical path change member 1103. The received light may be converted into image data and analyzed. In particular, when attachment of the tool 100 is identified by the attachment/detachment detection sensor 220, the control unit may control the projector 231 to emit output light toward the tool 100. The output light may illuminate the inside of the tool 100, and the camera 232 may acquire a clear image of the inside of the tool 100 due to the output light. That is, when the tool 100 is attached to the scanner body 200 and the output light of the projector 231 is emitted, the control unit may distinguish and determine whether the type of the tool 100 is the scanning tool 101 or the calibration tool 102, based on the image data acquired by the camera 232. By way of example, when it is determined that the image data acquired by the camera 232 indicates the shape of the pattern plate 1955 embedded in the calibration tool 102, the control unit may distinguish and determine that the tool 100 attached to the scanner body 200 is the calibration tool 102.

As another example, when it is determined that the image data acquired by the camera 232 does not indicate the shape of the pattern plate 1955, the control unit may distinguish and determine that the tool 100 attached to the scanner body 200 is a tool (e.g., the aforementioned scanning tool 101, and a protection tool (not shown) for protecting components of the scanner body 200) other than the calibration tool 102. In other words, the control unit may distinguish the calibration tool 102 from other tools 100 by detecting (or determining) the shape of the pattern plate 1955.

Further, the control unit may distinguish and determine whether the type of the tool 100 is an auto-calibration tool or a manual calibration tool, based on image data acquired by the camera 232.

The control unit may determine the type of the tool 100 attached to the scanner body 200 based on the image data acquired by the camera 232, and in particular, the control unit may perform a calibration operation when determining the tool 100 as the calibration tool 102.

According to the drawing in FIG. 10A, the image data acquired by the scanner body 200 having the scanning tool 101 attached thereto is displayed on the display unit 510. The image data acquired by the scanner body 200 having the scanning tool 101 attached thereto may represent the object image O.

According to the drawing in FIG. 10B, the image data acquired by the scanner body 200 having the calibration tool 102 attached thereto may represent the pattern plate 1955. The control unit may distinguish and detect whether the tool 100 attached to one end of the scanner body 200 is the scanning tool 101 or the calibration tool 102, based on the shape of the pattern plate 1955.

For example, when the shape of the image data is determined to represent the pattern plate 1955 of the calibration tool 102, the control unit may determine that the type of tool 100 coupled to the attachment/detachment surface 202 of the scanner body 200 corresponds to the calibration tool 102 and automatically perform a calibration operation. In addition, when the type of the tool 100 is determined to be the calibration tool 102, the control unit may automatically execute a calibration application on the display unit 510 to perform calibration. Further, when the type of the tool 100 is determined to be the calibration tool 102, the calibration application on the display may be automatically executed and a guide message for performing calibration may be provided to the user.

As another example, when the image data is determined to represent a shape different from that of the pattern plate 1955, the control unit may determine that the type of the tool 100 coupled to the attachment/detachment surface 202 of the scanner body 200 corresponds to the scanning tool 101, and perform a standby operation for acquiring two-dimensional image data and a three-dimensional model of the object O by an operation of the optical part 230 or perform a scanning operation.

Hereinafter, a method for the control unit of distinguishing the calibration tool 102 including the pattern plate 1955 from the scanning tool 101 will be described.

FIG. 11 is a view illustrating an example of identification targets 612 formed on the pattern plate 1955 of the calibration tool 102 and a sign 700 formed by the identification targets 612, and FIG. 12 is a view illustrating an example showing a process of performing calibration of the scanner body 200 by using the calibration tool 102. In FIG. 11, representation of the calibration targets 611 is omitted for convenience of explanation.

Referring to FIGS. 10B and 11, the pattern plate 1955 may include multiple targets 610. The multiple targets may be arranged according to a predetermined rule, at least some of the multiple targets may be used for calibration of the scanner body 200, and the others of the multiple targets may be used by the control unit to detect the type of the tool 100 attached to one end of the scanner body 200.

By way of example, the multiple targets 610 may include at least one calibration target 611 and multiple identification targets 612. The calibration target 611 may be used for calibration of the scanner body 200, and the calibration target 611 may be arranged in a predetermined array form. A configuration of the scanner body 200 is adjusted through the calibration target 611, and the scanner body 200 may be maintained to acquire a precise three-dimensional model of the object.

In addition, the identification targets 612 may be formed between calibration targets 611, and the multiple identification targets 612 may be arranged. The identification targets 612 may be spaced apart from the calibration target 611 and may have a shape different from that of the calibration target 611. For example, each of the identification targets 612 may have a circular shape having a larger diameter than the calibration target 611. However, the shape of the identification targets 612 is not limited to the described example, and each of the identification targets 612 may have a shape distinguished from that of the calibration target 611. Through the image data in which the identification target 612 is represented, the control unit may distinguish the type of the tool 100 attached to one end of the scanner body 200.

At least some of the multiple identification targets 612 may form a predetermined sign 700. As shown in FIG. 11, the predetermined sign 700 may be configured by connecting at least some of adjacent identification targets 612. For example, the sign 700 having a predetermined shape may be formed by connecting three identification targets 612. The sign 700 may have an “L” shape.

Further, for example, the sign 700 on the pattern plate 1955 may include a first sign 701, a second sign 702, a third sign 703, a fourth sign 704, and a fifth sign 705, and each of the signs 701, 702, 703, 704, and 705 may have first lengths h1, h2, h3, h4, and h5, respectively, and second lengths v1, v2, v3, v4, and v5, respectively. In this case, the first length may indicate a horizontal length between adjacent identification targets 612, and the second length may indicate a vertical length between adjacent identification targets 612. Here, the first length and the second length may be acquired based on the center of each of the identification targets 612, but are not limited to the presented reference.

The control unit may detect the calibration target 611, detect the identification targets 612, or detect the sign 700 formed by the identification targets 612 from the image data acquired by an operation of the camera, or detect the first length and/or the second length of the sign 700, so as to determine whether the tool 100 attached to the scanner body 200 is the calibration tool 102 including the pattern plate 1955. As such, by detecting the type of the tool 100 through at least one of the calibration target 611, the identification targets 612, the mark 700, or the dimension of the mark 700 from the image data, the control unit may quickly and accurately distinguish and determine the tool 100, thus having an advantage in that the calibration of the scanner body 200 may be easily performed accordingly.

Referring to FIG. 12, a calibration application screen 511 may be displayed on a display unit (e.g., 500 in FIG. 1). That is, the control unit may perform a calibration operation when determining that the tool 100 coupled to the scanner body 200 corresponds to the calibration tool 102.

Depending on cases, the control unit may automatically execute a calibration application when determining that the tool 100 coupled to the scanner body 200 corresponds to the calibration tool 102. That is, when determining that the tool 100 corresponds to the calibration tool 102, the control unit may execute the calibration application to be visually displayed on the display unit 510, and according to a calibration function built into the calibration application, may perform calibration of the scanner body 200. Accordingly, there is an advantage in that inconvenience is minimized for the user by omitting a separate application execution process and calibrating the scanner body 200 automatically.

Hereinafter, an embodiment in which the type of the tool 100 attached to one end of the scanner body 200 is identified and determined by the attachment/detachment detection sensor 220 will be described.

In an embodiment disclosed in the present disclosure, the distance d between the attachment/detachment detection sensor and the tool 100 may vary depending on the type of the tool 100. By way of example, the scanning tool 101 may have a protrusion (not shown) structure protruding inward toward the attachment/detachment detection sensor.

In this case, the control unit may determine the type of the tool 100 based on the distance d between the attachment/detachment detection sensor 220 and the tool 100 when the tool 100 is attached to the scanner body 200. The control unit may determine that the tool 100 corresponds to the scanning tool 101 when the distance d is equal to or less than a first distance threshold, and determine that the tool 100 corresponds to the calibration tool 102 when the distance d is less than or equal to a second distance threshold. The first distance threshold may be set to be smaller than the second distance threshold. When determining that the tool 100 corresponds to the calibration tool 102, the control unit may perform a calibration operation of the scanner body 200. As such, by distinguishing the type of the tool 100 based on the distance d acquired by measurement of the attachment/detachment detection sensor 220, there is an advantage in that the tool 100 may be quickly distinguished and quick calibration is possible when the calibration tool 102 is attached to the scanner body 200.

According to an embodiment disclosed in the present disclosure, an identifier such as a barcode or QR code may be marked on the tool 100, and the control unit may identify and determine the type of the tool 100 by recognizing the identifier marked on the tool 100 by using the camera 232 or a separate recognition unit (not shown).

Hereinafter, a process of detecting an auto-calibration tool and a manual calibration tool will be described.

FIG. 13A is an example illustrating an auto-calibration tool, and FIG. 13B is an example illustrating three-dimensional information of identification targets 612 which form a sign 700 of the auto-calibration tool. FIG. 14A is an example illustrating a manual calibration tool, and FIG. 14B is an example illustrating three-dimensional information of identification targets 612 which form a sign 700 of the manual calibration tool.

Referring to FIGS. 13A and 13B, the calibration tool 102 may include an auto-calibration tool. A mounting block 195 or 195a and a pattern plate 1955 or 1955a applied to the auto-calibration tool may be formed to be tilted in a first rotation direction. For example, the pattern plate 1955 or 1955a of the auto-calibration tool may be formed to be tilted by θ1 clockwise with respect to the y-axis on the coordinate space having an axis direction of z for the calibration tool 102. When the pattern plate 1955 or 1955a is formed to be tilted by θ1, image data acquired by the camera 2321 built in the scanner body 200 may also be tilted and acquired. Here, at least one of the first length or the second length of the sign 700 formed on the pattern plate 1955 or 1955a may be changed.

Referring to FIGS. 14A and 14B, the calibration tool 102 may include a manual calibration tool. A mounting block 195 or 195b and a pattern plate 1955 or 1955b applied to the manual calibration tool may be formed to be tilted in a second rotation direction. Here, the second rotation direction may be a rotation direction opposite to the first rotation direction. For example, the pattern plate 1955 or 1955b of the manual calibration tool may be formed to be tilted by θ2 counterclockwise with respect to the y-axis on the coordinate space having an axis direction of z for the calibration tool 102. When the pattern plate 1955 or 1955b is formed to be tilted by θ2, image data acquired by the camera 2321 built in the scanner body 200 may also be tilted and acquired. Here, at least one of the first length and the second length of the sign 700 formed on the pattern plate 1955 or 1955b may be changed.

FIGS. 15A and 15B are examples illustrating a process of performing calibration of a scanner body 200 by using a manual calibration tool. More specifically, FIG. 15A is for describing a process in which calibration of the scanner body 200 is performed when the pattern plate of the manual calibration tool is placed in a first location, and FIG. 15B is for describing a process of displaying a predetermined guide on a calibration application screen 511 to dispose the pattern plate of the manual calibration tool to a second location.

Referring to FIGS. 13A to 15B, the control unit may distinguish a type of a calibration tool 102 on the basis of image data acquired by photographing the pattern plate 1955. Specifically, the control unit determines the type of the calibration tool 102 as either a manual calibration tool or an auto-calibration tool based on the shape of the pattern plate 1955 included in the calibration tool 102, and may perform a calibration operation corresponding to the type of the tool 102.

By way of example, the control unit may distinguish whether the type of the calibration tool 102 is an auto-calibration tool or a manual calibration tool based on at least one of the first length or the second length of the sign 700. When the control unit distinguishes the type of the calibration tool 102, the control unit may apply a control signal corresponding to the type of the calibration tool 102 to the scanner body 200. For example, when the calibration tool 102 is an auto-calibration tool, the control unit may automatically perform calibration of the scanner body 200 by controlling at least one selected from the group of the scanner body 200 and the auto-calibration tool. For another example, when the calibration tool 102 is a manual calibration tool, the control unit may control a guide message for guiding the user to operate the manual calibration tool to be displayed on the application screen 511 on the display device 510, and the user may operate the manual calibration tool according to the guide message displayed on the application screen 511.

Referring to FIG. 15A as an example, a guide to place a dial of the calibration tool 102 at a first location may be displayed on the calibration application screen 511 under control of the control unit. When the user places the dial of the calibration tool 102 at the first location, a calibration process of the scanner body 200 at the first location is performed. Thereafter, referring to FIG. 15B as an example, a guide to place a dial of the calibration tool 102 at a second location may be displayed on the calibration application screen 511 under control of the control unit. When the user places the dial of the calibration tool 102 at the second location to move and/or rotate the pattern plate, a calibration process of the scanner body 200 at the second location is performed. The manual calibration tool may move or rotate the pattern plate to multiple locations (or angles) through the dial, and thus the calibration of the scanner body 200 may be performed.

As such, since the operation of the scanner corresponding to each tool is performed when one of the auto-calibration tool or the manual calibration tool is attached, there is an advantage in that the user may conveniently and quickly perform the calibration of the scanner body 200.

Hereinafter, another method for distinguishing an auto-calibration tool and a manual calibration tool will be described.

In the image processing device 1 according to an embodiment disclosed in the present disclosure, the scanner body 200 may include at least one camera 232, and multiple cameras 232 may be provided. Here, at least two pieces of image data may be acquired by the multiple cameras 232a and 232b. Depth information may be acquired based on the image data acquired by the multiple cameras 232a and 232b, that is, at least a portion of three-dimensional information of the calibration tool 102 may be acquired. For example, it is assumed that the identification targets 612 forming the sign 700 include a first identification target 612a, a second identification target 612b, and a third identification target 612c. In this case, tilting of the pattern plate 1955 causes a three-dimensional location of the first identification target 612a disposed on the auto-calibration tool to protrude by a predetermined thickness in the positive z-axis direction compared to those of the second identification target 612b and the third identification target 612c. In another example, tilting of the pattern plate 1955 causes a three-dimensional location of the first identification target 612a disposed on the manual calibration tool to be recessed by a predetermined depth in the negative z-axis direction compared to those of the second identification target 612b and the third identification target 612c.

Therefore, the user may distinguish the type of the calibration tool 102 based on the three-dimensional locational relationship of the identification targets 612, and the control unit may apply a control signal corresponding to the type of the calibration tool 102.

The foregoing description has been made that the pattern plate 1955 or 1955a of the auto-calibration tool is formed to be tilted clockwise with respect to the y-axis and the pattern plate 1955 or 1955b of the manual calibration tool is tilted counterclockwise with respect to the y-axis. However, the tilting directions of the auto-calibration tool and the manual calibration tool are not limited to the above, and it is also possible that the pattern plate 1955 or 1955a of the auto-calibration tool is tilted counterclockwise with respect to the y-axis and the pattern plate 1955 or 1955b of the manual calibration tool is formed to be tilted clockwise with respect to the y-axis.

The method for the control unit to distinguish the auto-calibration tool and the manual calibration tool is not limited to the above description. For example, when the auto-calibration tool and the manual calibration tool have different sign shapes on the pattern plate 1955, the control unit may distinguish and determine whether the calibration tool 102 coupled to the scanner body 200 is the auto-calibration tool or the manual calibration tool, based on the different sign shapes.

As another example, the control unit may distinguish and determine whether the calibration tool 102 coupled to the scanner body 200 is the auto-calibration tool or the manual calibration tool, based on the shape of the identification target (inclined shape of the identification target, size of the identification target, etc.) formed on the pattern plate 9155 of each of the auto-calibration tool or the manual calibration tool.

In the foregoing, the process of distinguishing/determining the scanning tool 101 and the calibration tool 102 by the control unit and/or the process of distinguishing/determining the automatic calibration tool and the manual calibration tool by the control unit may be performed through analysis of two-dimensional image data using artificial intelligence without acquiring three-dimensional information of the identification target.

Hereinafter, an image processing method using the image processing apparatus according to an embodiment of the present disclosure will be described. In describing the image processing method, descriptions of overlapping contents of the image processing apparatus according to an embodiment disclosed in the present disclosure will be briefly mentioned or omitted.

FIG. 16 is an exemplary flowchart illustrating an image processing method using an image processing apparatus according to the present disclosure.

Referring to FIG. 16, the image processing method using the image processing apparatus according to an embodiment disclosed in the present disclosure includes a tool attachment operation S110, a tool attachment/detachment identification operation S120, a tool distinguishing operation S130, and scanner control operation S140.

In the tool attachment operation S110, the user may mount a predetermined tool on the scanner body. For example, in the tool attachment operation S110, the tool attached to one end of the scanner body may include a calibration tool for calibration of the scanner body. The calibration tool may be formed in a form of a dark room with one side closed to block exit of light from the outside. As another example, the tool attached (coupled) to one end of the scanner body in the tool attachment operation S110 may include a scanning tool formed in a shape with one side open.

Meanwhile, in the tool attachment operation S110, the user may detach a tool previously attached and then mount a different tool from the detached tool. As an example, the user may detach the scanning tool attached to one end of the scanner body and mount the calibration tool on one end of the scanner body. By way of another example, the user may detach the calibration tool attached to one end of the scanner body and mount the scanning tool on one end of the scanner body.

In the tool attachment/detachment identification operation S120, the control unit may determine attachment/detachment of the tool to/from the scanner body. For example, the control unit may determine attachment/detachment of the tool to the scanner body based on a measurement of an attachment/detachment detection sensor which is formed on at least a portion of the scanner body and identifies detachment/attachment of the tool. For example, the attachment/detachment detection sensor may correspond to a proximity sensor, and the attachment/detachment detection sensor may measure and acquire a distance between the attachment/detachment detection sensor and a tool coupled to the scanner body.

When attachment/detachment of the tool to/from the scanner body is identified in the tool attachment/detachment identification operation S120, a tool distinguishing operation S130 may be performed. Here, the control unit may distinguish the type of the tool. A process for the control unit to distinguish the type of the tool in the tool distinguishing operation S130 will be described below.

After the control unit distinguishes the type of the tool attached to the scanner body by the tool distinguishing operation S130, the scanner control operation S140 may be performed. In the scanner control operation S140, the control unit may control an operation of the scanner body in response to the type of the distinguished tool. As an example, when the tool attached to the scanner body is distinguished as the calibration tool in the tool distinguishing operation S130, the control unit may control the calibration operation of the scanner body. As another example, when the tool attached to the scanner body is distinguished as the scanning tool in the tool distinguishing operation S130, the control unit may control the scan operation of the scanner body.

Hereinafter, an example of the tool distinguishing operation S120 of the image processing method using the image processing apparatus according to an embodiment of the present disclosure will be described.

FIG. 17 is an example illustrating the tool distinguishing operation S130 of the image processing method using the image processing apparatus according to the present disclosure.

Referring to FIGS. 16 and 17, in the tool distinguishing operation S130, the control unit may distinguish the type of the tool attached to one end of the scanner body by using the optical unit built into the scanner body. The optical part may include a projector for emitting predetermined output light toward the tool and at least one camera for receiving reflection light generated by reflection of the output light. As an example, when the output light reaches an object (e.g., inside the patient's oral cavity) disposed in the outside or a pattern plate embedded in a calibration tool, the output light is reflected from a surface (or inside) of the object or pattern plate to generate reflection light, and the reflection light may be guided to one end of the scanner body and received through a camera lens, which is a component of the camera. The received reflection light may be analyzed by an imaging board (more specifically, an image sensor included in the imaging board) electrically connected to the camera, and image data representing the object or pattern plate may be generated.

When the attachment of the tool to the scanner body is identified in the tool attachment/detachment identification operation S120, the control unit controls the projector to perform a light emission operation S1311 of emitting predetermined output light from the inside of the scanner body toward the tool side. For example, the output light may correspond to light in a visible light wavelength region, but is not necessarily limited to the corresponding wavelength region. If necessary, the output light may correspond to structured light generated through a pattern generation part (e.g., a pattern mask and/or DMD) included in the projector.

When output light is emitted in the light emission operation S1311, an image data acquisition operation S1312 may be performed. In the image data acquisition operation S1312, the camera built in the scanner body may acquire at least one piece of image data by receiving the reflection light generated by reflection of output light. The image data may correspond to at least one of two-dimensional data and three-dimensional data. For example, when the camera module includes two or more cameras (e.g., an L camera and an R camera), image data by each of the L camera and the R camera may be acquired.

When image data is acquired, a tool determination operation S1313 may be performed. In the tool determination operation S1313, the control unit may determine the type of the tool based on the acquired image data. As shown in FIG. 10A, if the acquired image data indicates a shape of the object, the control unit may determine the type of the tool attached to the scanner body as a scanning tool. In addition, if the acquired image data indicates a shape of the pattern plate, the control unit may determine the type of the tool attached to the scanner body as a calibration tool. That is, the control unit may determine the type of the tool by determining the shape of the pattern plate embedded in the calibration tool through the image data. In this case, the shape of the pattern plate identified and determined by the control unit through the image data may correspond to multiple targets included in the pattern plate.

The multiple targets may include at least one calibration target and multiple identification targets. The calibration target may be used for calibration of the scanner body. An identification target may be spaced apart from the calibration target, may have a shape different from that of the calibration target, and may be used to distinguish a type of a calibration tool. Further, in the tool determination operation S1313, the control unit may determine the type of the tool based on a predetermined sign formed by at least a portion of multiple identification targets.

By way of example, in the tool determination operation S1313, the control unit may determine a tool attached to the scanner body as a calibration tool when a calibration target and/or an identification target exist in the acquired image data. As another example, in the tool determination operation S1313, the control unit may determine a tool attached to the scanner body as a calibration tool when a sign exists in the acquired image data. As yet another example, in the tool determination operation S1313, the control unit may determine a tool attached to the scanner body as a calibration tool based on the shape and size of a sign or the shape and size of an identification target existing in the acquired image data. However, the elements listed in the embodiments disclosed in the present disclosure to describe a reference for determining the type of the tool are exemplary, and other references may be applied as needed.

Hereinafter, another example of the tool distinguishing operation S130 for distinguishing a tool attached to the scanner body will be described.

FIG. 18 is another example illustrating the tool distinguishing operation S130 of the image processing method using the image processing apparatus according to the present disclosure.

Referring to FIGS. 16 and 18, in the tool distinguishing operation S130, the control unit may detect the type of the tool based on a distance between the attachment/detachment detection sensor and the tool when the tool is attached to the scanner body. The control unit may distinguish and determine that the tool corresponds to the scanning tool when the distance is equal to or less than a first distance threshold, and distinguish and determine that the tool corresponds to the calibration tool when the distance is less than or equal to a second distance threshold. The first distance threshold may be set to be smaller than the second distance threshold, but the first distance threshold may be set to be higher than the second distance threshold, if necessary.

In the tool distinguishing operation S130 performed according to another example of the present disclosure, the distance measurement operation S1321 may be performed. In the distance measurement operation S1321, the attachment/detachment detection sensor formed on at least a portion of the scanner body may measure the distance between the tool and the scanner body when the tool is attached to one end of the scanner body. The distance may correspond to a vertical distance from the attachment/detachment detection sensor to the inner surface of the tool, but is not limited thereto.

Thereafter, the tool determination operation S1322 may be performed. In the tool determination operation S1322, the control unit detects and determines the type of the tool based on the distance between the tool and the scanner body acquired by the attachment/detachment detection sensor. The process of determining the type of the tool based on the distance between the tool and the scanner body is the same as the above description, and thus a detailed description thereof will be omitted.

Hereinafter, a detailed operation of the scanner control operation S140 will be described.

FIG. 19 is an example illustrating the scanner control operation S140 of the image processing method using the image processing apparatus according to the present disclosure.

Referring to FIGS. 16 and 19, a tool type identification operation S141 may be performed. In the tool type identification operation S141, the control unit may identify whether the tool determined in the tool distinguishing operation S130 is a calibration tool.

After the tool-type identification operation S141 is performed, a calibration application execution operation S142 may be performed. More specifically, when the control unit detects and distinguishes the tool as a calibration tool, the control unit may automatically execute a calibration application. Accordingly, the calibration of the scanner body may be performed by the executed calibration application.

When the control unit distinguishes the type of the tool as a calibration tool, a calibration performance operation S143 may be performed. The control unit may control the scanner body to perform a calibration operation in the scanner control operation S140. The calibration may represent correcting a configuration of at least some (particularly, the optical part) of the components built into the scanner body.

Meanwhile, when the tool attached to the scanner body is identified as a scanning tool in the tool type identification operation S141, a scanning operation S144 may be performed. It may be determined that the scanning tool attached to the scanner body is attached by the user to scan the object, and the control unit controls the optical part so that the scanner body may acquire a three-dimensional model and/or an image representing the object.

Hereinafter, a detailed operation of the calibration performance operation S143 will be described.

FIG. 20 is an example illustrating the calibration performance operation S143 of the image processing method using the image processing apparatus according to the present disclosure.

Referring to FIGS. 16, 19, and 20, a calibration process may be different depending on whether the calibration tool is an auto-calibration tool or a manual calibration tool. Accordingly, the calibration performance operation S143 may include a calibration tool identification operation S1431. In the calibration tool identification operation S1431, the control unit may identify a type of the calibration tool that has been distinguished, detected, and/or determined in the tool distinguishing operation S130 or the tool determination operation S1313 or S1322, which is detailed operation of the tool distinguishing operation. For example, the calibration tool may correspond to an auto-calibration tool that automatically moves and/or rotates the pattern plate, or a manual calibration tool that allows the user to move and/or rotate the pattern plate using a dial lever.

Meanwhile, the auto-calibration tool and the manual calibration tool may be distinguished according to the tool determination operation S1313 of determining the type of the tool based on image data. As an example, the pattern plates included in the auto-calibration tool and the manual calibration tool may be formed to be tilted at different angles. The auto-calibration tool and the manual calibration tool may be distinguished based on at least one of a difference in sizes of identification targets that appear when the pattern plate is formed to be tilted at different angles, or a difference in shapes of predetermined signs formed by at least some of the identification targets (e.g., at least one of the first length and the second length of the sign, or the shape and size or the entire sign itself).

Further, as described above, when acquiring three-dimensional information of identification targets included in the pattern plate, the auto-calibration tool and the manual calibration tool may be distinguished based on three-dimensional locations of the identification targets.

When the control unit identifies the tool as the manual calibration tool in the tool distinguishing operation S130 and the control unit identifies the calibration tool as the manual calibration tool in the calibration tool identification operation S1431, a manual calibration performance operation S1434 may be performed. In the manual calibration performance operation S1434 of the scan operation control operation S140, the control unit may generate an instruction for guiding the user to operate the manual calibration tool. For example, the control unit may control the calibration application screen 511 to be displayed on the display unit 510, and as shown in FIG. 15B, the user may operate the manual calibration tool to induce calibration of the scanner body to proceed.

On the other hand, when the control unit identifies the tool as the auto-calibration tool in the tool distinguishing operation S130 and the control unit identifies the calibration tool as the auto-calibration tool in the calibration tool identification operation S1431, an auto-calibration performance operation S1433 may be performed. In the auto-calibration performance operation S1433 of the scan operation control operation S140, the control unit may control at least one selected from the group of the scanner body and the auto-calibration tool so that the scanner body automatically performs a calibration operation. In this case, the auto-calibration tool may operate so that the pattern plate is automatically moved and/or rotated according to the calibration process of the scanner body by the above-described pattern moving part. Accordingly, the calibration of the scanner body may be automatically performed.

Depending on cases, the pattern plate built into the auto-calibration tool may not be placed in an initial location thereof. Therefore, the calibration performance operation S143 of the image processing method according to an embodiment disclosed in the present disclosure may further include a pattern plate location operation S1432. In the pattern plate location operation S1432, the control unit may control the pattern plate built into the auto-calibration tool to be located in the initial location before the calibration operation (more specifically, the auto-calibration operation S1433) of the scanner control operation S140 is performed.

For example, initial location information of the pattern plate may be stored in the external electronic device and/or the scanner body. Therefore, in case that the auto-calibration tool is attached to the scanner body, the control unit may apply a control signal to the auto-calibration tool so as to control a location of the pattern plate to be the initial location thereof. Accordingly, there is an advantage in that accurate calibration of the scanner body may be performed through the auto-calibration tool.

The image processing method using the image processing apparatus according to various embodiments of the present disclosure may be implemented in a form of program command to be executed through various computer means and recorded on a computer readable medium. In addition, an embodiment of the present disclosure may correspond to a computer-readable recording medium on which one or more programs including commands for executing the calibration process of the scanner body are recorded.

The computer readable medium may include program commands, data files, data structures, etc. alone or in combination. Program commands recorded on the medium may be specially designed and configured for the present disclosure or known and usable to those skilled in computer software. Examples of computer-readable recording media include magnetic media such as hard disks, floppy disks, and magnetic tapes, optical media such as CD-ROMs and DVDs, magneto-optical media such as floptical disks, and hardware apparatuses specially configured to store and execute program commands, such as a ROM, a RAM, a flash memory, and the like. Examples of program commands include high-level language code that may be executed by a computer using an interpreter, as well as machine language code produced by a compiler.

Here, a device-readable storage medium may be provided in the form of a non-transitory storage medium. In this case, the term “non-temporary” merely means that the storage medium is a tangible device and does not include a signal (e.g., electromagnetic wave), and the term does not distinguish the case where data is stored semi-permanently in the storage medium and the case where data is stored temporarily. For example, the term “non-temporary storage medium” may include a buffer in which data is temporarily stored.

According to an embodiment, the image processing method using the image processing apparatus according to various embodiments disclosed herein may be included in a computer program product, and provided. A computer program product may be traded between sellers and buyers as a commodity. The computer program product may be distributed in the form of a machine-readable storage medium (e.g., a compact disc read only memory (CD-ROM)), or distributed on-line (e.g., downloaded or uploaded) through an application store (e.g., Play Store™) or directly between two user devices (e.g., smartphones). In the case of on-line distribution, at least a portion of the computer program product (e.g., a downloadable app) may be stored, or at least temporarily generated, on a device-readable storage medium, such as a manufacturer's server, an application store's server, or the memory of a relay server.

Specifically, the image processing method using the image processing apparatus according to various embodiments disclosed herein may be implemented as a computer program product including a recording medium for storing a program to perform an operation of acquiring a multilingual sentence and an operation of acquiring vector values corresponding to each of words included in the multilingual sentence by using a multilingual translation model, converting the acquired vector values to vector values corresponding to a target language, and based on the converted vector values, acquiring a sentence including the target language.

It is to be understood that the image processing apparatus, the image processing method using the image processing apparatus, and the recording medium according to the embodiments disclosed in the present disclosure mutually share the aforementioned advantages.

The above description is merely an example of the technical ideas of the disclosure, and various modifications and variations will be apparent to those skilled in the art to which the disclosure belongs, without departing from the essential features of the disclosure.

Therefore, the embodiments disclosed in the present disclosure are intended to illustrate, not limit, the technical ideas of the present disclosure, and the scope of the technical ideas of the present disclosure is not limited by these embodiments. The scope of protection of the present disclosure should be construed in accordance with the following claims, and all technical ideas within the scope of the equivalents should be construed to be included within the scope of the present disclosure.

An embodiment disclosed in the present disclosure provides an image processing apparatus and an image processing method using the image processing apparatus for maintaining a high scanning precision of a scanner body by performing a corresponding operation of the scanner body according to a type of a tool detachably coupled to the scanner body.

1. An image processing apparatus comprising:
a scanner body;
a tool detachably coupled to one end of the scanner body; and
a control unit configured to control an operation of the scanner body in response to the tool.

2. The image processing apparatus of claim 1, wherein the tool comprises either one of:
a calibration tool formed into a shape with one side closed and configured to calibrate the scanner body; or
a scanning tool formed into a shape with one side open.

3. The image processing apparatus of claim 1, wherein the scanner body comprises:
an attachment/detachment detection sensor formed on at least a portion of the scanner body and configured to identify whether the tool is attached to or detached from an attachment/detachment surface which couples the tool to the scanner body;
a projector configured to emit a predetermined output light toward the tool; and
at least one camera configured to receive reflection light generated by reflection of the output light and acquire at least one piece of image data from the reflection light.

4. The image processing apparatus of claim 3, wherein the projector is configured to, when attachment or detachment of the tool is identified by the attachment/detachment detection sensor, emit the output light.

5. The image processing apparatus of claim 3, wherein the control unit is configured to determine a type of the tool attached to the scanner body based on the image data acquired by the camera, and when the tool is determined as a calibration tool, perform a calibration operation.

6. The image processing apparatus of claim 5, wherein the control unit is configured to, when a shape of the image data is determined to represent a pattern plate of the calibration tool, determine the type of the tool as the calibration tool and perform a calibration operation.

7. The image processing apparatus of claim 6, wherein the control unit is configured to determine a type of the calibration tool as either a manual calibration tool or an auto-calibration tool based on the shape of the pattern plate, and perform a calibration operation corresponding to the type of the calibration tool.

8. The image processing apparatus of claim 3, wherein the control unit is configured to,
when the tool is attached to the scanner body, determine a type of the tool based on a distance between the attachment/detachment detection sensor and the tool, and when the tool is determined as a calibration tool, perform a calibration operation.

9. The image processing apparatus of claim 1, wherein the control unit is configured to, when the tool is determined as a calibration tool, automatically execute a calibration application to be displayed on a display unit.

10. The image processing apparatus of claim 9, wherein the control unit is configured to, when the tool is determined as a manual calibration tool among the calibration tool, control a guide message to be output on the display unit to guide a user to operate the manual calibration tool.

11. The image processing apparatus of claim 3, wherein the calibration tool further comprises a pattern plate configured to be tiltable within a predetermined angle range based on a longitudinal direction of the scanner body, and
wherein the pattern plate comprises multiple targets.

12. The image processing apparatus of claim 11, wherein the multiple targets comprise:
at least one calibration target for calibration of the scanner body; and
multiple identification targets formed to be spaced apart from the calibration target, having a shape different from that of the calibration target, and used to identify a type of the calibration tool.

13. An image processing method using an image processing apparatus, the method comprising:
detecting, by a control unit of the image processing apparatus, attachment of a tool to a scanner body of the image processing apparatus;
distinguishing, by the control unit, a type of the tool; and
controlling, by the control unit, an operation of the scanner body in response to the type of the tool.

14. The method of claim 13, wherein the detecting comprises: identifying the attachment of the tool with an attachment detection sensor formed on an attachment surface which is formed on at least a portion of the scanner body and configured to couple the tool to the scanner body.

15. The method of claim 14, wherein the distinguishing comprises:
when the attachment of the tool is identified, emitting predetermined output light from a projector built into the scanner body;
acquiring at least one piece of image data by at least one camera built into the scanner body receiving reflection light generated by reflection of the output light; and
determining a type of the tool based on the image data.

16. The method of claim 15, wherein the determining comprises: determining, by the control unit, the type of the tool by determining a shape of a pattern plate built into a calibration tool through the image data.

17. The method of claim 14, further comprising: when the tool is attached to the scanner body, detecting, by the control unit, a type of the tool based on a distance between the attachment detection sensor and the tool.

18. The method of claim 13, further comprising: when the tool is determined as a calibration tool, automatically executing, by the control unit, a calibration application to be displayed on a display unit.

19. The method of claim 13, wherein the controlling comprises: when a type of the tool distinguished as a manual calibration tool among the calibration tool, controlling, by the control unit, a guide message to be output on a display unit to guide the user to operate the manual calibration tool.

20. The method of claim 13, wherein the controlling comprises: when a type of the tool distinguished as an auto-calibration tool among the calibration tool, controlling, by the control unit, at least one selected from the group of the scanner body and the auto-calibration tool to automatically perform a calibration operation.
